(19) United States
(12) Patent Application Publication
(10) Pub. No.: US 2024/0180975 A1
(43) Pub. Date: Jun. 6, 2024

(54) BACTERIAL STRAINS FOR USE AS PROBIOTICS, COMPOSITIONS THEREOF, DEPOSITED STRAINS AND METHOD TO IDENTIFY PROBIOTIC BACTERIAL STRAINS

(71) Applicant: INSTITUT PASTEUR, PARIS CEDEX 15 (FR)

(72) Inventors: JEAN-MARC GHIGO, FONTENAY AUX ROSES (FR); DAVID PEREZ-PASCUAL, JOUY EN JOSAS (FR); FRANZISKA STRESSMANN, BERLIN (DE); JOAQUIN BERNAL-BAYARD, Seville (ES)

(21) Appl. No.: 17/996,127

(22) PCT Filed: Apr. 15, 2021
(86) PCT No.: PCT/IB2021/000280

§ 371 Date: Oct. 13, 2022

(30) Foreign Application Priority Data
Apr. 15, 2020 (EP) 20169704.2

(51) Int. Cl. A61K 35/741; A23K 10/18; A23K 50/80; C12N 1/20; C12R 1/01; C12R 1/20

(52) U.S. Cl. CPC A61K 35/741; A23K 10/18; A23K 50/80; C12N 1/205; C12R 2001/01; C12R 2001/20

(57) ABSTRACT

The invention relates to a bacterial strain or a combination of bacterial strains, selected from the group consisting of: a Chryseobacterium massiliae strain, a Flavobacterium sp. strain with at least 95% or more Average Nucleotide Identity (ANI) value with the Flavobacterium sp. strain whose genome comprises SEQ ID NO: 1 or as identified by Accession Number No. I-5481 deposited at the Collection Nationale De Culture De Microorganismes (CNCM) on Jan. 24, 2020, and variants thereof, for use as a probiotic in fish(es). The bacterial strain(s) may has(ve) at least 95% or more Average Nucleotide Identity (ANI) value with the Chryseobacterium massiliae strain whose genome comprises SEQ ID NO:2 or as identified by Accession Number No. I-5479 deposited at the CNCM on Jan. 24, 2020, and/or the Flavobacterium sp. strain whose genome comprises SEQ ID NO: 1 or as identified by Accession Number No. I-5481 deposited at the CNCM on Jan. 24, 2020, respectively, or variants thereof. Probiotic use may be directed to preventing or minimizing infections by Flavobacterium columnare in fishes. The invention also concerns the said deposited bacterial strains, or probiotic compositions or food products or kits comprising the same, and a method to identify bacterial strain(s) that are probiotic against a pathogen infection.

### FIELD OF THE INVENTION

The invention relates to the field of probiotics, especially under the form of bacterial strains as active ingredients for use as probiotics or suitable for the design of probiotic compositions for administration to fishes. The invention thus involves using such active ingredients for treatment of said fishes, for preventive and/or beneficial effect on their health. The active ingredients of the present invention have in particular been shown to be beneficial against infection challenge by Flavobacterium columnare bacterium. The invention also relates to a method to identify probiotic bacterial strains.

### BACKGROUND OF THE INVENTION

Animal resident microbial consortia form complex and long-term associations playing important community-level functions essential for host development and physiology [1,2]. Microbiota ecosystems also provide protection against exogenous pathogens by a combination of inhibition of pathogen settlement and growth and/or stimulation of the host immune system [3-7]. From the perspective of microbial community composition, a shift or reduction in resident microbial diversity, a phenomenon generally referred to as dysbiosis, is often associated with increased susceptibility to infection due to the loss or change in abundance of key microbial community members [7, 8]. These observations early supported the notion that addition or promotion of individually or communally protective bacteria (such as probiotics) could minimize microbiota dysbiosis or directly prevent infection to restore host health [9-11].

Although the efficacy of probiotics have been shown in animal and humans, their mechanisms of action are poorly understood and diversity surveys or low throughput experimental models offer limited information on the contribution of species to community functions [1, 6, 12-14]. Moreover, characterization of bacterial strains improving colonization resistance is still hindered by the complexity of host-commensal ecosystems. Zebrafish have recently emerged as a powerful tool to study microbe-microbe and host-microbe interactions [15-20]. Zebrafish can be easily reared germ-free or gnotobiotically in association with specific bacterial species [15,21]. Moreover, zebrafish bacterial communities are increasingly well characterized and a number of phylogenetically distinct zebrafish gut bacteria can be cultured, making this model system directly amenable to microbiota manipulation and assessment of probiotic effect on host infection resistance [22-25]. Several studies have used zebrafish to evaluate the effect of exogenous addition of potential probiotics on host resistance to infection [23-25]. However, whereas various lactic acid bacteria (Lactobacilli spp., Bacillus spp.) were shown to improve the outcome of infection against a number of zebrafish pathogens (e.g. Aeromonas hydrophila, A. veronii, Streptococcus agalactiae and Vibrio parahaemolyticus) [26-30], the reported protections were often partial, illustrating the difficulty in identifying fully protective exogenous probiotics.

The inventors used germ-free and conventional zebrafish larvae to mine the indigenous commensal microbiota for bacterial species protecting against Flavobacterium columnare, a bacteroidetes pathogenic bacterium affecting wild and cultured fish species, including carp, channel catfish, goldfish, eel, salmonids and tilapia [31, 32]. They identified two infection resistance scenarios preventing mortality caused by F. columnare, mediated either by the bacteroidetes bacterium Chryseobacterium massiliae or by an assembly of 9 otherwise non-protecting bacterial species that formed a protective community. Their results constitute a powerful approach to mine host microbiota and identify key members mediating colonization resistance, providing insight into how to engineering microbial communities to protect against pathogens in aquaculture settings and beyond.

Actually, while wild fish stocks are approaching biologically unsustainable limits, fish aquaculture is a fast-growing industry providing over half of all consumed fish [101]. However, intensive aquaculture promotes pathogens outbreaks and the high mortality rate in aquaculture facilities constitutes an important bottleneck for fish production [102-104]. These health issues primarily affect immunologically immature young fish larvae, in which vaccination is unpractical [105, 106], prompting the prophylactic and therapeutic use of antibiotics and chemical disinfectants to prevent fish diseases [107-109]. However, the widespread use of antibiotics and chemical disinfectants are associated with final consumer safety risks, environmental pollution and spread of antibiotic resistance resulting in broad human health concerns [104, 110]. In this context, use of probiotic to improve fish health and protect disease-susceptible juveniles appears as an economically and ecologically sensible alternative strategy to antibiotics treatments [111-113].

Probiotics are live microorganisms conferring health benefit on the host via mechanisms, including promotion of growth, immunostimulation or direct inhibition of pathogenic microorganisms [114-116]. Considering the important protective role played by host microbiota against pathogenic microorganism, a process known as colonization resistance [117-119], the fish-associated microbial community is considered an interesting source of probiotic bacteria [120-122]. Indeed, gastrointestinal tract and fish mucus are the most common sources of potential fish probiotics [123, 124]. However, selection of probiotic bacteria, is often empirical or hampered by the lack of repeatability and reproducibility of in vivo challenges, often performed in relatively poorly controlled conditions and high inter-individual microbial composition [121, 125].

To circumvent the experimental difficulties associated with evidence-based identification of fish probiotics, use of the germ-free or fully controlled gnotobiotic host is a promising strategy [126, 127] and several economically important fish species have been successfully reared under sterile conditions, including the Atlantic cod (Gadus morhua L.) [128]. Atlantic halibut (Hippoglossus hippoglossus) [129], European sea bass (Dicentrarchus labrax) or turbot (Scophthalmus maximus) (for review, see [131, 132]). Other studies used germ free or gnotobiotically reared laboratory zebrafish (Danio rerio) to evaluate the effect of addition of exogenous probiotic on host infection [133-136]. In most cases, however, the tested probiotics were exogenous to the host and short-term microbiota residents, only providing partial protection against the tested pathogens.

The inventors thus studied the potential of members of the rainbow trout (Oncorhynchus mykiss) microbiota to protect against infection by Flavobacterium columnare, a fish pathogen causing major losses in aquaculture fish species, especially catfish and salmonids [137]. Using a new protocol to rear trout larvae in sterile conditions, they showed that germ-free but not conventional trout larvae were extremely sensitive to infection by F. columnare. They then used reconventionalization of germ-free trout to identify bacterial species originating from trout (Flavobacterium sp.) or zebrafish (Chryseobacterioum massiliae) microbiota that fully restored protection against F. columnare infection. Their results show that their new gnotobiotic trout model enables mining of teleostean fish microbiota to rationally identify fish probiotics that could, alone or in combination contribute to protect against columnaris disease in rainbow trout and other fish in the context of aquaculture research and husbandry.

The invention therefore relies on the experiments described herein, and proposes new means and tools for addressing the above-mentioned problems. In particular, the invention relates to the provision of relevant probiotic material for protection of fishes, against pathogens and their deleterious effects.

### DETAILED DESCRIPTION OF THE INVENTION

The invention therefore relates to a bacterial strain or combination of bacterial strains, wherein the bacterial strain or at least one bacterial strain of the combination is selected from the group consisting of: a Chryseobacterium massiliae strain, a Chryseobacterium massiliae strain wherein one or more virulence factor coding gene(s) and/or antibiotic resistance gene(s) is(are) deleted or inactivated, a Flavobacterium sp. strain whose genome has at least 95% or more Average Nucleotide Identity (ANI) with SEQ ID NO:1 or which has at least 95% ANI with the Flavobacterium sp. strain identified by Accession Number No. I-5481 deposited at the CNCM on Jan. 24, 2020, and a Flavobacterium sp. strain whose genome has at least 95% or more Average Nucleotide Identity (ANI) with SEQ ID NO:1 or which has at least 95% ANI with the Flavobacterium sp. strain identified by Accession Number No. I-5481 deposited at the CNCM on Jan. 24, 2020 wherein one or more virulence factor coding gene(s) and/or antibiotic resistance genes is(are) deleted or inactivated, for use as a probiotic in a fish or population(s) of fishes.

The address of CNCM is: Collection Nationale de Culture de Microorganismes, Institut Pasteur, 28 rue du Dr Roux, 75724 Paris CEDEX 15, France.

SEQ ID NO:1 is the whole genome sequence of a particular Flavobacterium. sp. strain described herein, sequenced according to the experiments described herein and also available in ENA (European Nucleotide Archive) database under primary accession number ERS4574862 (version 1) and secondary accession number SAMEA6847264 (Tax ID 2730889, scientific name Flavobacterium sp. UGB 4466).

The 16S rRNA gene sequence of the particular Flavobacterium. sp. strain of SEQ ID NO: 1 is disclosed herein under SEQ ID NO: 3.

The expressions “16S rRNA” and “16S rDNA” are used interchangeably herein,

It will be understood that references to either “fish” (singular) or “fishes” (plural) in the present text, the latter generally referring to populations of fishes, are meant to be interchangeable throughout the present description, unless the context or general sense dictate otherwise.

Average Nucleotide Identity (ANI) value can be readily determined by the skilled person using common knowledge and available tools, which are well detailed in the literature. The ANI between two genomes, especially prokaryotic genomes, is commonly known as a taxonomic method for classification that emerged in the era of genomics. Before that, DNA-DNA hybridization (DDH) has been used for nearly 50 years as a standard for prokaryotic species circumscriptions at the genomic level. Other methods are available as well, some of which are also detailed in the present application, including in the Experimental Section, such as 16S rRNA gene sequence similarity, the recA gene sequence similarity or the rplC gene sequence similarity. Present description also provides bibliographic references regarding implementation of the 16S rRNA gene sequence similarity method, in addition to the guidance provided herein, which can be used as guidelines by the skilled person.

Software tools for carrying out the Average Nucleotide Identity (ANI) method and calculating an ANI value are also readily accessible to the skilled person: they can in particular be freely accessible over the internet. An example can be found at https://www.ezbiocloud.net/tools/ani. The literature provides details regarding available tools. In particular, identity percentages can conventionally be calculated through local, preferably global, sequence alignment algorithms and their available computerized implementations. In a most preferred embodiment, identity percentages are calculated over the entire length of the compared sequences, which may be the entire genomes of the compared strains. Global alignments, which attempt to align every residue in every sequence, are most useful when the sequences in the query set are similar and of roughly equal size. Computerized implementations of the algorithms used are generally associated with default parameters in the literature, which can be used for running said algorithm. The skilled person can readily adapt the same taking into account its objective or the sequences comparison made.

Whatever the algorithm used, it is however admitted that an ANI value of 95% is an appropriate cutoff value for distinguishing between two species, i.e., for classifying the genome of the strain whose classification is sought, within an existing species (as annotated in databases) or to define a new, unknown to date, species.

The identification of the specific bacterial strains of the present invention, as described herein, was carried out by whole genome-based identification, and was performed by the TrueBac ID system (v1.92, DB:20190603) [https://www.truebacid.com/; [66]. The main section of the TrueBac ID-Genome system consists of (1) a proprietary reference database, named the TrueBac database, which is curated to hold up-to-date nomenclature, 16S rRNA gene, and genome sequences of type/reference strains, and (2) the optimized bioinformatics pipeline that provides the identification of a query genome sequence using the average nucleotide identity (ANI). The algorithmic identification scheme using WGS works as follow: first, the most phylogenetically closely related pool of taxa was identified using a search of three genes—16S rRNA, recA, and rplC—which were extracted from the whole genome assembly. Then, to the gene-based searches, the Mash tool (https://github.com/marbl/mash) is used for additional fast whole-genome based searches. The top-hits of the above four searches are then pooled, and the ANI was calculated using the MUMmer tool. Species-level identification was performed based on the algorithmic cut-off set at 95% ANI when possible or when the 16S rDNA gene sequence similarity was >99% (for identifying the species with accuracy, resulting, in turn, in the identification of a novel species of Flavobacterium strain).

The skilled person can therefore readily determine the ANI value using the MUMmer tool described above and herein.

According to a particular embodiment, a Chryseobacterium massiliae strain as described herein or of use within the present invention has, alternatively or in addition of any other feature as described herein, a genome that has at least 95% or more, preferably 96% or more, Average Nucleotide Identity (ANI) value with SEQ ID NO: 2.

SEQ ID NO:2 is the whole genome sequence of a particular Chryseobacterium massiliae strain described herein, sequenced according to the experiments described herein and also available in ENA (European Nucleotide Archive) database under primary accession number ERS4385998 (version 1) and secondary accession number SAMEA6623857 (Tax ID 204089, scientific name Candidatus Chryseobacterium massiliae).

In a particular embodiment, the strain is a variant of the above mentioned strain, in which one or more virulence factor coding gene(s) and/or antibiotic resistance genes is(are) deleted or inactivated.

The 16S rRNA gene sequence of the particular Chryseobacterium massiliae strain of SEQ ID NO: 2 is disclosed herein under SEQ ID NO: 4.

The closest hit taxons of the particular strains as described herein are provided in the tables below, for the particular Flavobacterium. Sp and the Chryseobacterium massiliae strains described herein, respectively.

| No. | Hit taxon | ANI (%) | ANI coverage (%) | 16S (%) | recA (%) | rpIC (%) |
|---|---|---|---|---|---|---|
| 1 | Flavobacterium | 94.65 | 82.4 | 97.80 | 98.51 | 99.52 |
| | spartansii | | | | | |
| 2 | Flavobacterium | 94.62 | 83.9 | 97.80 | 98.11 | 99.68 |
| | tructae | | | | | |
| 3 | Flavobacterium | 85.26 | 39.8 | 97.27 | 89.48 | N/A |
| | chilense | | | | | |

It can thus be seen that the Flavobacterium. Sp bacterial strain of the invention (also termed Flavobacterium. Sp 4466 herein) has 94.65% Average Nucleotide Identity (ANI) with the Flavobacterium spartansii ATCC BAA-2541 strain, whose sequence is available in the GenBank database under accession number MUHG01000041.1 (version 1) -https://www.ncbi.nlm.nih.gov/nuccore/MUHG01000041.1. The strain whose sequence is disclosed in SEQ ID NO: 1 accordingly constitutes a novel Flavobacterium species, never described so far.

Accordingly, in particular embodiments the present invention concerns or makes use of Flavobacterium. sp. strains with at least 95% or more Average Nucleotide Identity (ANI) value with the Flavobacterium spartansii ATCC BAA-2541 bacterial strain taken as a reference sequence. Such strains may be used, as described herein, as a probiotic in a fish or population(s) of fishes.

The particular Flavobacterium. Sp bacterial strain described herein also has 97.80% 16S rRNA gene sequence similarity with the Flavobacterium spartansii ATCC BAA-2541 bacterial strain.

The 16S rRNA gene sequence of Flavobacterium spartansii ATCC BAA-2541 bacterial strain is disclosed herein under SEQ ID NO: 5.

Accordingly, in particular embodiments the present invention concerns or makes use of Flavobacterium. sp. strains whose genome comprises a 16s rDNA sequence with at least 98% sequence identity to SEQ ID NO: 5 taken as a reference sequence.

According to a particular embodiment, the present invention concerns or makes use of Flavobacterium. sp. strains whose genome have at least 95% or more Average Nucleotide Identity (ANI) value with the Flavobacterium spartansii ATCC BAA-2541 bacterial strain taken as a reference sequence and comprising a 16s rDNA sequence with at least 98% sequence identity to SEQ ID NO: 5 taken as a reference sequence.

In a particular embodiment, the strain is a variant of any one of the above mentioned strain, in which one or more virulence factor coding gene(s) and/or antibiotic resistance genes is(are) deleted or inactivated.

In a particular embodiment, a Flavobacterium. sp. strain of the invention or as described in any embodiment herein keeps the properties, in particular the biological properties as described herein, of the Flavobacterium sp. strain identified by Accession Number No. I-5481 deposited at the Collection Nationale de Culture de Microorganismes (CNCM) on Jan. 24, 2020, and/or keeps the the properties, in particular the biological properties as described herein, of the Flavobacterium sp. strain of SEQ ID NO:1. Probiotic properties are particularly referred to.

| No. | Hit taxon | ANI (%) | ANI coverage (%) | 16S (%) | recA (%) | rpIC (%) |
|---|---|---|---|---|---|---|
| 1 | Chryseobacterium | 95.85 | 83.1 | 99.86 | 96.61 | 99.84 |
| | massiliae | | | | | |
| 2 | Chryseobacterium | 83.81 | 21 | 97.95 | N/A | N/A |
| | defluvii | | | | | |
| 3 | Chryseobacterium | 84.30 | 4.1 | 97.67 | N/A | N/A |
| | zeae | | | | | |

It can thus be seen that the Chryseobacterium massiliae bacterial strain of the invention has 95.85% Average Nucleotide Identity (ANI) with the Chryseobacterium massiliae CCUG 51329 strain, whose sequence is available in the GenBank database under accession number ASM338553 (version 1)—https://www.ncbi.nlm.nih.gov/assembly/GCF_003385535.1/.

Accordingly, in particular embodiments the present invention concerns or makes use of Chryseobacterium massiliae strains whose genome have at least 96% or more Average Nucleotide Identity (ANI) value with the Chryseobacterium massiliae CCUG 51329 bacterial strain taken as a reference sequence. Such strains may be used, as described herein, as a probiotic in a fish or population(s) of fishes.

The Chryseobacterium massiliae bacterial strain described herein also has 99.86% 16S rRNA gene sequence similarity with the Chryseobacterium massiliae CCUG 51329 bacterial strain.

The 16S rRNA gene sequence of Chryseobacterium massiliae CCUG 51329 bacterial strain is disclosed herein under SEQ ID NO:6.

According to a particular embodiment, the present invention concerns or makes use of Chryseobacterium massiliae strains with at least 96% or more Average Nucleotide Identity (ANI) value with the Chryseobacterium massiliae CCUG 51329 bacterial strain taken as a reference sequence and comprising a 16s rDNA sequence with at least 99.9% sequence identity to SEQ ID NO: 6 taken as a reference sequence.

Alternatively, the present invention concerns or makes use of Chryseobacterium massiliae strains whose genomes have at least 97% or more Average Nucleotide Identity (ANI) value with the Chryseobacterium massiliae CCUG 51329 bacterial strain taken as a reference sequence.

In a particular embodiment, the strain is a variant of any one of the above mentioned strain, in which one or more virulence factor coding gene(s) and/or antibiotic resistance genes is(are) deleted or inactivated.

In a particular embodiment, a Chryseobacterium massiliae strain of the invention or as described in any embodiment herein, including variants, keeps the properties, in particular the biological properties as described herein, of the Chryseobacterium massiliae strain identified by Accession Number No. I-5479 deposited at the Collection Nationale de Culture de Microorganismes (CNCM) on Jan. 24, 2020, and/or keeps the properties, in particular the biological properties as described herein, of the Chryseobacterium massiliae strain of SEQ ID NO:2. Probiotic properties are particularly referred to.

According to a particular embodiment, the ANI value of a Flavobacterium. sp. strain of use within instant invention, with the Flavobacterium sp. strain identified by Accession Number No. I-5481 deposited at the Collection Nationale De Culture De Microorganismes (CNCM) on Jan. 24, 2020, is either as disclosed in any embodiment herein, and/or can reach 96% or more, 97% or more, 98% or more, 99% or more, or be 100%.

Other methods than the ANI value for defining Flavobacterium. sp encompassed by instant invention are detailed herein. The skilled person will appreciate that all these methods can readily define appropriate subsets of Flavobacterium. sp strains in the context of instant invention. Table 1 below show examples of correlating data in the course of verification of the taxonomic identities of prokaryotic genomes, although classification methods are different.

As defined herein, the strains of use within instant invention may be defined through a series of parameters, e.g., an ANI value of 95% or more and a so-called “16S” value of 97%, 98% or 99% or more. These combinations are encompassed herein by the use of the wording “and/or” for combining parameters, according to all values and embodiments described herein.

According to a particular embodiment, a Chryseobacterium massiliae strain of use within instant invention, is a Chryseobacterium massiliae strain with at least 95% or more Average Nucleotide Identity (ANI) value with the Chryseobacterium massiliae strain identified by Accession Number No. I-5479 deposited at the Collection Nationale De Culture De Microorganismes (CNCM) on Jan. 24, 2020, and/or a Chryseobacterium massiliae with at least 95% or more, preferably 96% or more, Average Nucleotide Identity (ANI) value with SEQ ID NO:2.

When reference is made to the fact that one or more virulence factor coding gene(s) or antibiotic resistance gene(s) is(are) deleted or inactivated in the considered strain, which is a feature that can be associated to any embodiment described herein, it is observed that the skilled person in the art can readily select the one or more genes which are deleted or inactivated, and if required engineer the resulting strain, following guidance as presented herein and his general knowledge regarding bacterial strain attenuation or inactivation of resistance gene(s).

According to an embodiment, a bacterial strain of the invention, according to any embodiment described herein, is avirulent to a healthy fish. The said bacterial strain can be naturally avirulent, or it can be avirulent by genetic and/or chemical attenuation.

Methods for attenuation of pathogenic bacteria are known in the art. Genetic attenuation can be achieved by inactivating one or more gene(s) involved in metabolic pathway(s) of the bacteria, more particularly in one or more pathogenic mechanism(s) of the bacteria, and/or by inactivating one or more gene(s) involved in or responsible for the production of virulence factor(s) of the bacteria.

According to an embodiment, the bacterial strain is attenuated by partial or complete deletion of one or more gene(s), still more particularly by partial or complete deletion of one or more genes involved in or responsible for the production of virulence factor(s) of said bacterial strain. Such genes may be as listed in Tables 7 and 8 of present description (see Results section), for each respectively considered strain.

More precisely, the isolated strain Chryseobacterium sp. has been shown to contain five predicted virulence factors, including some proteins involved in capsule biosynthesis, heat shock protein HtpB subunit, KatA catalase and ClpP protease proteolytic subunit. According to a particular embodiment, the Chryseobacterium sp. of the invention has one or several genes amongst those it contains (as it can be determined following the guidance and tools provided and described in the results and methods sections herein), especially those having the above referred involvements, which are partially or fully deleted.

The Flavobacterium sp. 4466 has been shown to contain genes encoding for capsule, sialic acid synthase, type IV and type VI secretion systems effectors and catalase as potential virulence factors. According to a particular embodiment, the Flavobacterium sp. 4466 strain of the invention has one or several genes amongst those it contains (as it can be determined following the guidance and tools provided and described in the results and methods sections herein), especially those having the above referred involvements, which are partially or fully deleted.

For all these embodiments, exemplary representative genes are provided in Table 6, the content of which is referred to herein: any one or several of the genes precisely cited in this Table can be found deleted or partially deleted in a variant strain of the invention.

The same applies to antibiotic resistance genes. According to an embodiment, which can be cumulative to the other embodiments described in present application, the bacterial strain is engineered by partial or complete deletion of one or more genes, still more particularly by partial or complete deletion of one or more genes involved in antibiotic resistance of said bacterial strain. Such genes may be as listed in Table 6 of present description (see Results section), for each respectively considered strain.

The Flavobacterium sp. 4466 has been shown to contain genes encoding resistance to carbapenem, lincosamide, streptogramin, pleuromutilin, and fluoroquinolone antibiotics. According to a particular embodiment, the Flavobacterium sp. 4466 strain of the invention has one or several genes amongst those it contains (as it can be determined following the guidance and tools provided and described in the results and methods sections herein), especially those having the above referred involvements, which are partially or fully deleted.

In all matters, partial deletion is achieved to an extent sufficient to inactivate the function of the gene.

For ease of writing in the presence description, bacterial strains with one or more virulence factor coding gene(s) or antibiotic resistance genes is(are) deleted or inactivated can also be termed “variants” herein. Variants of the invention however keep the functional properties of their parents strains. In particular they keep their probiotic effect.

Unless the context or general sense dictate otherwise, when reference to bacterial strain(s) is made in present description, variants thereof as defined above, are systematically encompassed in the definition of said bacterial strain or combination or association of strains (where the variant of one strain can be found associated either with other non variant strain(s), or with other variant strain(s), according to all possible combinations thereof).

According to a particular embodiment, applicable throughout the description, the bacterial strain or bacterial strains (and variants thereof) is(are) associated with acceptable carrier or delivery vehicle(s) and optionally adjuvant component(s), within a single composition or separate compositions comprising or consisting essentially of, or consisting of, a mixture of distinct bacterial strains. According to a particular embodiment, applicable throughout the description, the bacterial strain or bacterial strains of interest within the present invention is(are) used for the purpose described herein, or in the compositions described herein, to the proviso that they keep it/their probiotic properties, especially vis à vis/against any application described herein, in particular against infections by pathogen(s) and their outcomes as described herein, in particular when the Flavobacterium columnare bacteria is considered, and/or vis à vis/against a fish or a population of fishes to be treated as described in any embodiment of the present description.

According to a particular embodiment, when only one bacterial strain is used, said bacterial strain is administered to a host in need thereof either without acceptable carrier or delivery vehicle(s) (or adjuvant component(s) when relevant), or within a composition as defined above and according to any embodiment described in the present description.

According to a particular embodiment, when a combination of distinct bacterial strains is used, said bacterial strains are administered to a host in need thereof:
i. as separate bacterial strains without acceptable carrier or delivery vehicle(s) (or adjuvant component(s) when relevant), or
ii. as a mixture of distinct bacterial strains found within a single composition, or
iii. within distinct compositions each comprising, or consisting essentially of, or consisting of, at least one bacterial strain, or
iv. as a collocation of at least one individualized (singled out) bacterial strain and distinct composition(s) each comprising, or consisting essentially of, or consisting of, at least one bacterial strain.

By “individualized”, or alternatively “singled out” bacterial strain, it is meant a bacterial strain without acceptable carrier or delivery vehicle(s) (or adjuvant component(s) when relevant.

The skilled person can readily understand that bacterial strain(s) of the invention, with desirable protective properties as described herein, may be directly put into contact with the fish or population of fishes to serve as a probiotic. For instance, the bacterial strain(s) of the invention may be directly added into the raising water of said fish.

Pharmaceutical products can also be devised. Examples of pharmaceuticals for the delivery of probiotics are: capsules, liquids, powder beads, tablets . . . . In a particular embodiment, bacterial strain(s) of the invention is(are) administered in a liquid formulation. In a particular embodiment, bacterial strain(s) of the invention is(are) administered in a powder formulation.

Alternatively, bacterial strain(s) of the invention may be conveniently, as known in the field, be provided to the fish(es) so as to achieve its probiotic effect, along with meals, or when contained within food products: bacterial strain(s) of the invention may be provided to fishes along with fish meals (i.e., in association with: provided at the same time but not mixed with the fish meal before the fishes are fed), or contained within fish food products. In a particular embodiment, bacterial strain(s) of the invention is(are) encapsulated.

In a particular embodiment, bacterial strain(s) of the invention is(are) administered as and when found in a food-based product, or is(are) administered in association with a food-based product. Carrier or delivery vehicle(s) as mentioned herein are adapted accordingly, following the conventional practice in the field.

According to a particular embodiment, probiotic effect is achieved when the bacterial strain(s) of the invention is(are) delivered to the gastro-intestinal tract (GIT) of the fishes to be treated. Instant application demonstrates colonisation of the gastro-intestinal tract of treated fishes.

According to a particular embodiment, probiotic effect is achieved when the bacterial strain(s) are alive (live bacteria), or at least in a viable state, or capable of being rehydrated and/or revived and/or revitalized so as to be alive when put in contact with the intended host.

According to a particular embodiment, applicable throughout the description, bacterial strain(s) of the invention is(are) live bacterium(a).

Nothwistanding the above and according to a particular embodiment, bacterial strain(s) of the invention is(are) lyophilized. Lyophilized commercial preparations have advantages for storage and transport.

In a particular embodiment, bacterial strain(s) of the invention are encapsulated. Probiotics encapsulation is also a convenient mean to enhance probiotics stability, facilitate handling and storage of probiotic cultures and to protect the bacteria from detrimental conditions (e.g., oxygen contact, freezing temperature(s) or acidic environment) during production, storage and gastrointestinal transit when relevant. It may also be adapted for administration of bacteria directly into fishes raising water. By “encapsulated” it is therefore meant that the bacteria have been submitted to an encapsulation process defined as a process by which cells, especially live cells, are packaged within a shell material to offer protection against unfavourable environmental conditions and possibly (not mandatorily) allowing for their controlled release under intestinal conditions. Several methods are known in the art for encapsulation of probiotics, such as spray drying, extrusion, emulsion or phase separation, freeze drying, ionotropic gelation, etc. This list is not limitative. Probiotic encapsulation technology (PET) commonly allows for microorganisms immobilization within semipermeable and/or biocompatible materials. The literature readily provides to the skilled person examples and guidance regarding encapsulation, see, for example, Prado et al. Applied Microbiology and Biotechnology volume 104, pages 1993-2006(2020) doi.org/10.1007/s00253-019-10332-0, or Amir et al. Fish & Shellfish Immunology, Volume 95. December 2019, Pages 464-472 doi.org/10.1016/j.fsi.2019.11.011, or Hai N.V., Journal of Applied Microbiology 119, 917-935, 2015, doi.org/10.1111/jam.12886. Accordingly, carrier or delivery vehicle(s) as mentioned herein encompass those allowing for encapsulation of bacterial strain(s) of the invention.

Conversely, a bacterial strain or combination of bacterial strains of the invention, may be found in a composition, wherein the said composition consists essentially of, or consists of, at least one bacterial strain and an acceptable carrier or delivery vehicle(s) and optionally adjuvant component(s).

Such a composition may allow the bacterial strain or combination of bacterial strains of the invention, according to all embodiments described herein, to be found as a formulation adapted for the intended purpose, with carrier or delivery vehicle(s) chosen to meet the formulation requirements. Formulations can encompass: liquid or powder formulations, capsules, powder beads, tablets, encapsulated bacterial strain(s) as described above, appropriately formulated (e.g., within a liquid), and/or food-products(s) when food is added.

It is to be understood that the skilled person can readily determine suitable carrier(s) or delivery vehicle(s) to be found in such a “composition” or “formulation”; Details are provided above and herein.

According to a particular embodiment, applicable throughout the description, when a combination of distinct bacterial strains is used, said bacterial strains are administered to a host in need thereof simultaneously or separately in any order, or sequentially in any order.

According to a particular embodiment, applicable throughout the present description, the bacterial strain(s) of the present invention are isolated bacterial strain(s).

By “probiotic” it is meant that the bacterial strain(s) or combination thereof have the capability of exerting a beneficial effect on the organism to which they are administered, preferably a beneficial effect on the health status. Probiotics are organisms, which when they are administered to an host, especially but not exclusively as a food ingredient, or as an add-on to a food ingredient (therefore not necessarily strictly administered as a meal) confer a health benefit to the host. The beneficial effects may be achieved through interactions of the bacterial strain(s) or combination thereof in the context of the invention, with the microbiota of the host to which they are administered. Accordingly, the “probiotic” feature used as an adjective to qualify the bacterial strain(s), active ingredient(s), composition(s), and other food or liquid product(s) described herein, means that the same has the intended functionality encompassed by the “probiotic” definition.

In a particular aspect, colonization of gastrointestinal tract by the bacterial strains of the invention has been demonstrated herein, as well as protection and increased survival of the host after administration of the bacterial strains of the invention, as beneficial effects.

According to a particular embodiment, “probiotic” or “probiotic effect” means the capability to colonize the gastrointestinal tract of the host to which the probiotic is administered.

According to another particular embodiment, which can be in addition to other features, including the above feature, “probiotic” or “probiotic effect” means the capability to protect the host to which the probiotic is administered, from the deleterious effect on health status from an infection by another pathogen, such as the F. columnare pathogen. In this respect deleterious effects on health status resulting from an infection by the F. columnare pathogen are thoroughly described herein. Any of the effects reversing, even partly, the deleterious effects of the same on the host, can be said to participate to a probiotic effect or constitute a probiotic effect. The skilled person can therefore determine whether a probiotic effect is achieved, by comparison to an absence of treatment, by observing the obtained beneficial effects on health status.

According to another particular embodiment, which can be in addition to other features, including the above features, “probiotic” or “probiotic effect” means the capability to confer increased survival of the host or population of hosts after administration of the bacterial strains of the invention. The experimental section herein also provides guidance regarding how to evaluate whether increased survival is conferred to an host or population of hosts after administration of the bacterial strains of the invention. The skilled person can therefore determine whether a probiotic effect is achieved according to this criteria, by comparison to an absence of treatment, by observing the increased survival effect obtained on the host or population of hosts. The data provided herein shows that the invention confers beneficial effects to the hosts to which it is administered, on their health, and increase their survival, even after challenge by a pathogen.

According to the invention, the host is a fish, in particular a teleostean fish (i.e., a fish of the Teleostei infraclass), preferably a fish that may be affected by or affected by columnariosis diseases.

In a particular embodiment, the fish is selected amongst one or several of the species listed in the below table: those species are commonly cultivated fishes.

| Common name | Binomial name |
|---|---|
| Grass carp | Ctenopharyngodon idella |
| Peruvian anchoveta | Engraulis ringens |
| Silver carp | Hypophthalmichthys molitrix |
| Common carp | Cyprinus carpio |
| Asari, | Venerupis philippinarum |
| Japanese littleneck, | |
| Manila clam, | |
| Filipino Venus, | |
| Japanese cockle, | |
| Japanese carpet shell | |
| Alaska pollock | Theragra chalcogramma |
| Nile tilapia | Oreochromis niloticus |
| Whiteleg shrimp | Penaeus vannamei |
| Bighead carp | Hypophthalmichthys nobilis |
| Skipjack tuna | Katsuwonus pelamis |
| Catla | Catla catla |
| Crucian carp | Carassius carassius |
| Atlantic salmon | Salmo salar |
| Atlantic herring | Clupea harengus |
| Chub mackerel | Scomber japonicus |
| Rohu | Labeo rohita |
| Yellowfin tuna | Thunnus albacares |
| Japanese anchovy | Engraulis japonicus |
| Largehead hairtail | Trichiurus lepturus |
| Atlantic cod | Gadus morhua |
| European pilchard | Sardina pilchardus |
| Capelin | Mailotus villosus |
| Jumbo flying squid | Dosidicus gigas |
| Milkfish | Chanos chanos |
| Atlantic mackerel | Scomber scombrus |
| Rainbow trout | Oncorhynchus mykiss |
| Giant tiger prawn | Penaeus monodon |
| Asian tiger shrimp | |
| Araucanian herring | Clupea bentincki |
| Chinese razor clam | Sinonovacula constricta |
| Agemaki clam | |
| Chinese mitten crab | Eriocheir sinensis |
| Wuchang bream | Megalobrama amblycephala |
| Pacific oyster | Crassostrea gigas |
| Japanese oyster | |
| Miyagi oyster | |
| Louisiana crawfish | Procambarus clarkil |
| Red swamp crawfish | |
| Akiami paste shrimp | Acetes japonicus |
| Gulf menhaden | Brevoortia patronus |
| Indian oil sardine | Sardinella longiceps |
| Black carp | Mylopharyngodon piceus |
| European anchovy | Engraulis encrasicolus |
| Northern snakehead | Channa argus |
| Pacific cod | Gadus macrocephalus |
| Pacific saury | Cololabis saira |
| Pacific herring | Clupea pallasii |
| Bigeye tuna | Thunnus obesus |
| Chilean jack mackerel | Trachurus murphyi |
| Yellow croaker | Larimichthys polyactis |
| Haddock | Melanogrammus aeglefinus |
| Gazami crab | Portunus trituberculatus |
| Amur catfish | Silurus asotus |
| Japanese common | |
| catfish | |
| European sprat | Sprattus sprattus |
| Pink salmon | Oncorhynchus gorbuscha |
| Mrigal carp | Cirrhinus mrigala |
| Channel catfish | Ictalurus punctatus |
| Blood cockle | Anadara granosa |
| Blue whiting | Micromesistius poutassou |
| Hilsa shad | Tenualosa ilisha |
| Daggertooth pike conger | Muraenesox cinereus |
| California pilchard | Sardinops caeruleus |
| Cape horse mackerel | Trachurus capensis |
| Pacific anchoveta | Cetengraulis mysticetus |
| Japanese flying squid | Todarodes pacificus |
| Argentine shortfin squid | Illex argentinus |
| Pollock | Pollachius virens |
| Chinese softshell turtle | Pelodiscus sinensis |
| Kawakawa | Euthynnus affinis |
| Indian mackerel | Rastrelliger kanagurta |
| Asian swamp eel | Monopterus albus |
| Yesso scallop | Patinopecten yessoensis |
| Argentine hake | Merluccius hubbsi |
| Northern prawn | Pandalus borealis |
| Short mackerel | Rastrelliger brachysoma |
| Southern rough shrimp | Trachysalambria curvirostris |
| Southern African | Engraulis capensis |
| anchovy | |
| Pond loach | Misgurnus anguillicaudatus |
| Iridescent shark | Pangasius hypophthalmus |
| Mandarin fish | Siniperca chuatsi |
| Chinese perch | |
| Nile perch | Lates niloticus |
| Round sardinella | Sardinella aurita |
| Japanese pilchard | Sardinops melanostictus |
| American sea scallop | Placopecten magellanicus |
| Bombay-duck | Harpadon nehereus |
| Yellowhead catfish | Tachysurus fulvidraco |
| Korean bullhead | |
| Narrow-barred Spanish | Scomberomorus commerson |
| mackerel | |
| Albacore | Thunnus alalunga |
| Madeiran sardinella | Sardinella maderensis |
| Bonga shad | Ethmalosa fimbriata |
| Silver cyprinid | Rastrineobola argentea |
| Nile tilapia | Oreochromis niloticus |
| Longtail tuna | Thunnus tonggol |
| Atlantic menhaden | Brevoortia tyrannus |
| Giant tiger prawn | Penaeus monodon |
| North Pacific hake | Merluccius productus |
| Atlantic horse mackerel | Trachurus trachurus |
| Japanese jack mackerel | Trachurus japonicus |
| Pacific thread herring | Opisthonema libertate |
| Bigeye scad | Selar crumenophthalmus |
| Yellowstripe scad | Selaroides leptolepis |
| Chum salmon | Oncorhynchus keta |
| Antarctic krill | Euphausia superba |
| Blue swimming crab | Portunus pelagicus |
| Pacific sand lance | Ammodytes personatus |
| Pacific sandlance | |
| Goldstripe sardinella | Sardinella gibbosa |

According to a particular embodiment, the fish is selected amongst: eels (Anguilla sp.), salmonids (Oncorhynchus sp. and Salmo sp.), tilapia (Oreochromis sp.), hybrid-striped bass (Morone chrysops x M. saxatilis), walleye (Stitzostedion vitreum), channel catfish, cetrachids (such as largemouth bass (Micropterus salmoides)), bait minnows (Pimephales promelas), goldfish (Carassisu auratus), carp (Cyprinus carpio) and aquarium fish (tropical fish species such as black mollies (Poecilia sphenops)) and platies (Xiphophorus maculatus).

Such fishes are commonly known as being target fish species of F. columnare pathogen.

According to a particular embodiment, the fish is a rainbow trout (synonym for Oncorhynchus mykiss, throughout the present description).

According to a particular embodiment, the treated fish is at any stage of its development. It may for example be an egg, a larvae especially without a fully mature/developed immune system, an adult, including an adult with or without a fully mature/developed immune system. The fish may be immune-compromised.

According to a preferred embodiment, the treated fish is within a population of treated fishes that includes larvae, or the treated animal is a fish larvae.

In a particular embodiment, the treated host is a fish larvae.

According to a particular embodiment, the treated fish is a rainbow trout and is within a population of treated rainbow trout that includes larvae, or is a rainbow trout larvae.

Accordingly, in a particular embodiment, the fish (or some of the fishes) is a rainbow trout at larval stage of development.

According to a particular embodiment, the treated fish is found in aquaculture settings or fish husbandry settings.

As stated above, the strains of use within instant invention may be defined through a single parameter or a series of parameters that applies cumulatively, e.g., an ANI value and/or a so-called “16S” value, and may be furthered by a functional definition.

According to a particular embodiment, bacterial strain(s), including when found in a combination according to the invention, keep the property(ies), especially the biological property(ies), more specifically the probiotic property(ies), of the natural counterpart(s) from which they derive. The property(ies) of said natural counterpart(s) can be readily determined by the tests disclosed in the experimental section of the present description. Determination of whether or not bacterial strain(s) of instant invention keep the said property(ies) can be readily made by comparison, or by a comparative assay, or by an entire assay following the guidance provided herein.

According to a particular embodiment, the bacterial strain(s), including when found in a combination according to the invention, is(are) selected from bacterial strain(s) that comprise(s) a 16s rDNA sequence with at least 97% sequence identity, in particular at least 98% or at least a 99% sequence identity, to a 16s rDNA sequence present in the Chryseobacterium massiliae strain identified by Accession Number No. I-5479 deposited at the CNCM on Jan. 24, 2020, or the Flavobacterium. sp strain identified by Accession Number No. I-5481 deposited at the CNCM on Jan. 24. 2020, respectively.

Cumulatively or alternatively to the above, the bacterial strain(s), including when found in a combination according to the invention, is(are) selected from bacterial strain(s) that comprise(s) a 16s rDNA sequence with at least 97% sequence identity to a 16s rDNA sequence present in a reference sequence of Chryseobacterium massiliae or Flavobacterium. sp strain, respectively.

Examples of references sequences are described above: they may be Chryseobacterium massiliae CCUG 51329 and Flavobacterium spartani ATCC BAA-2541, respectively. Percentages can be as described in any embodiment or paragraph described herein.

Cumulatively or alternatively to the above, the bacterial strain(s), including when found in a combination according to the invention, is(are) selected from bacterial strain(s) that comprise(s) a 16s rDNA sequence with at least 97% sequence identity to SEQ ID NO:3, or SEQ ID NO:4, respectively. These are the sequences corresponding to the 16s rDNA sequences as found in SEQ ID NO:1 and SEQ ID NO:2, respectively, the boundaries of the said 16s rDNA sequence being readily determinable to the skilled person by reference to the annotations found in databases entries, and by common knowledge.

According to particular embodiments, the sequence identity using a so-called 16s rDNA sequence taxonomic classification tool can reach 98% or more, 99% or more, or be 100%.

The skilled person has knowledge regarding how to implement 16s rDNA sequence taxonomic classification and in particular determine an at least 97% sequence identity, as this technique is well documented in the literature and guidance is provided herein (see for instance discussed herein, and the experimental section).

The literature provides details regarding the particularities of 16s rDNA sequence taxonomic classification. In particular, identity percentages can conventionally be calculated through local, preferably global, sequence alignment algorithms and their available computerized implementations. In a most preferred embodiment, identity percentages are calculated over the entire length of the compared 16 s rDNA sequences. Global alignments, which attempt to align every residue in every sequence, are most useful when the sequences in the query set are similar and of roughly equal size. Computerized implementations of the algorithms used are generally associated with default parameters in the literature, which can be used for running said alogorithm. The skilled person can readily adapt the same taking into account its objective or the sequences comparison made.

Whatever the algorithm used, it is however admitted that a value of 97% identity between 16s rDNA sequences is an appropriate cutoff value for distinguishing between two species, i.e., for classifying the genome of the strain whose classification is sought, within an existing species (as annotated in databases) or to define a new, unknown to date, species.

According to a particular embodiment, the bacterial strain(s), including when found in a combination according to the invention, is(are) selected from bacterial strain(s) that:is(are) from the group consisting of: a Chryseobacterium massiliae strain identified by Accession Number No. I-5479 deposited at the CNCM on Jan. 24, 2020 (SEQ ID NO: 2) and a Flavobacterium sp. strain identified by Accession Number No. I-5481 deposited at the CNCM on Jan. 24, 2020 (SEQ ID NO: 1).

Throughout the present application, CNCM stands for Collection Nationale de Cultures de Microorganismes (Institut Pasteur, 25 rue du Docteur Roux, F-75724 Paris Cedex 15, France).

According to a particular embodiment, the invention relates to a Chryseobacterium massiliae bacterial strain, especially the Chryseobacterium massiliae strain identified by Accession Number No. I-5479 deposited at the CNCM on Jan. 24, 2020 (SEQ ID NO:2), for use as a probiotic.

The Chryseobacterium massiliae bacterial strain with Accession Number No. I-5479 is also known the the CNCM under Identification reference UGB 3610. This bacterial strain has been isolated from zebrafish. An example of suitable growth medium is a combination of Tryptone Yeast Extract Salts and R2A Agar (Reasoner's 2A Agar). Suggested incubation conditions are: 28° C., aerobie, 250 r.p.m. (revolution per minute) shaking.

According to a particular embodiment, the invention relates to a Flavobacterium sp. bacterial strain, especially the Flavobacterium sp. strain identified by Accession Number No. I-5481 deposited at the CNCM on Jan. 24, 2020 (SEQ ID NO:1), for use as a probiotic.

The Flavobacterium sp. bacterial strain with Accession Number No. I-5481 is also known the the CNCM under Identification reference UGB 4466. This bacterial strain has been isolated from rainbow trout. An example of suitable growth medium is R2A Agar (Reasoner's 2A Agar). Suggested incubation conditions are: 20° C., aerobie, 250 r.p.m. (revolution per minute) shaking.

According to a particular embodiment, the bacterial strain or combination of bacterial strains is for use as defined herein so that the at least one bacterial strain(s) is(are) administered to:
a. a host that is a fish, in particular a teleostean fish, more particularly a rainbow trout, especially larvae thereof, or
b. hosts comprising or consisting essentially of or consisting of homogenous or mixed population(s) of fishes, for example a population(s) encompassing fishes such as rainbow trout at distinct stages of their development or growth.

By “homogenous” population(s) of fishes, it is meant population(s) of fishes whose individuals essentially pertain to the same species, or strictly pertain to the same species. According to a particular aspect, such “homogenous” population(s) may encompass fishes at different stages of their development, or fishes with sensibly the same developmental stage.

Conversely, by “mixed” population(s) of fishes, it is meant population(s) of fishes whose individuals pertain to distinct species, i.e., encompass distinct fish species. According to a particular aspect, such “mixed” population(s) may still encompass fishes at different stages of their development, or fishes with sensibly the same developmental stage.

The developmental stage of fishes may be: eggs, larvae, juvenile fish, adults without or with a mature immune system.

According to a particular embodiment, the developmental stage of a fish administered with the active ingredient(s) of instant invention is the larval developmental stage (in case of a population of fishes, it means that said population encompasses fish larvae, at least in part, or entirely). Basically, the larval stage lasts from hatching up to the juvenile stage, defined as the moment where all fin rays are present on the fish and the growth of fish scales has started (squamation). A key event for defining the end of the larval stage and the beginning of the juvenile stage is when the notochord associated with the tail fin on the ventral side of the spinal cord develops flexion (becomes flexible). Of note, the larval stage can be further subdivided into preflexion, flexion, and postflexion stages, or subdivided between the yolk-sac larval stage and transformation stage, which follows absorption of the yolk-sac (the latter ending the yolk-sac larval stage). Although these provisions are not strict so that juvenile fish or fishes in other developmental stage, including adults, cannot be administered with the active ingredient(s) described herein, it is observed that since fishes vaccination is commonly known not to be efficient or relevant at the larval stage, instant invention provides a clear advance to the art when fishes in the larval stage are treated, i.e., fishes at a stage where vaccination is thought to fail.

In a particular aspect applicable throughout the description, a combination as defined herein may additionally comprise at least one another bacterial strain of the indigenous microbiota of the treated host species or a host species within the treated population of hosts.

Examples of associations of several bacterial strains from the indigenous microbiota of a fish can be:
With respect to the rainbow trout: Delftia acidovorans, Flavobacterium sp, Aeromonas rivipollensis, Pseudomonas helmanticensis, Aeromonas rivipollensis, Pseudomonas baetica, Aeromonas hydrophyla, Flavobacterium plurextorum, Acinetobacter sp., Flavobacterium plurextorum and Pseudomonas sp.
With respect to the zebrafish (Danio rerio throughout the present description): Chryseobacterium massiliae. Aeromonas veronii, Pseudomonas mossellii, Stenotrophomas maltophilia, Aeromonas caviae, Pseudomonas peli, Pseudomonas sediminis, Phyllobacterium myrsinacearum, Pseudomonas nitroreducens.

According to another aspect, the bacterial strain(s) or combination thereof of the present invention are further suitable, as probiotics or in themselves, for (one or several amongst the following list, according to all possible combinations thereof):
a. preventing the occurrence of an infection by the Flavobacterium columnare pathogen in the host(s) fish species or treated population of hosts fish species,
b. preventing the occurrence of a further infection, or minimizing the consequences of an ongoing infection, by the Flavobacterium columnare pathogen in the host(s) fish species or treated population of hosts fish species, and/or
c. enhancing the resistance against the Flavobacterium columnare pathogen of the treated host(s) fish species or treated population of hosts fish species, and/or
d. preventing or minimizing the extent (spread and consequences) of subsequent infections by the Flavobacterium columnare pathogen in the host(s) fish species or treated population of hosts fish species, or enhancing the resistance against the Flavobacterium columnare pathogen of the treated host(s) fish species or treated population of hosts fish species, and/or
d. preventing or controlling diseases, notably caused by an infection with the Flavobacterium columnare pathogen, in the treated host(s) fish species or treated population of hosts fish species, especially preventing or minimizing disease propagation within the treated population of hosts fish species, and/or
e. increasing the lifespan of treated host(s) fish species or population of treated hosts fish species, or reducing the mortality of treated host(s) species or population of treated hosts fish species.

The invention concerns, as a “pathogen” constantly, or regularly, or susceptible to be present, in aquaculture settings, the Flavobacterium columnare pathogen, pertaining to the Flavobacterium genus. Information about that pathogen can for example be found in “Columnaris Disease in Fish: A Review With Emphasis on Bacterium-Host Interactions” (DOI: 10.1186/1297-9716-44-27, Declerq et al. Veterinary Research 2013, 44:27)

Infections of fishes with Flavobacterium columnare may arise in the context of seasonal diseases outbreaks in aquaculture farming settings: these infections arising in the context of seasonal outbreaks are also infections of interest in the context of the present invention.

Instant invention concerns, according to a particular aspect, the prevention of occurrence(s) of infection of a fish, by the Flavobacterium columnare pathogen. In this aspect, the administration of active ingredient(s) of the invention are conceived in a prophylactic manner, the host not having been infected by the pathogen.

As used herein, “infection” means the invasion and multiplication of the pathogen in the host's body. It will be appreciated that an infection may cause or not symptoms, being subclinical or clinically apparent, respectively An infection may remain localized, or it may spread through the blood or lymphatic vessels to become systemic (bodywide)

Whether the considered fish has already been infected or is at risk of being infected by a Flavobacterium columnare pathogen, and whether the concerned fish is asymptomatic or symptomatic for an infection by Flavobacterium columnare, instant invention also concerns enhancement of the resistance of the considered fish against the Flavobacterium columnare pathogen.

According to another aspect, the invention also concerns the prevention and/or control of diseases, notably caused by an infection with the Flavobacterium columnare pathogen, i.e., a disease whose initial cause is the existence of an infection with this pathogen, or a disease who is known to be caused by or is known to be strongly correlated with a pre-infection of the host by this pathogen (according to a cause-consequence relationship).

Therefore according to a particular embodiment, the bacterial strain(s) or combination thereof of the present invention are used against diseases caused (or induced, or resulting of) by infections by the Flavobacterium columnare bacteria, the disease being in particular columnaris disease.

The most known disease directly caused by an infection with Flavobacterium columnare, columnaris disease (also spelled Columnariosis disease in the literature).

According to a particular embodiment, the invention aims at mitigating rainbow trout fish diseases, especially columnaris disease (also termed Columnariosis disease in the literature). In this context and according to a particular embodiment, the disease is columnaris disease, so that the invention is aimed at mitigating columnaris diseases in fishes affected Flavobacterium columnare. When the fish is a rainbow trout, the invention is is aimed at mitigating rainbow trout columnaris disease.

By “increasing the lifespan of treated host(s) species or population of treated hosts species” it is conversely meant “reducing the mortality of treated host(s) species or population of treated hosts species”, since it will be understood that the active ingredients of present invention have a beneficial effect on the hosts organisms so that comorbidity events resulting from pathogens infections are minimized or alleviated, at the level of the single individual and/or at the level of a whole population of hosts. The mortality reduction can be readily assessed by the skilled person by observation of the treated host(s) species or population of treated hosts' species. The lifespan increase can also be readily assessed by the skilled person in light of the prior occurrence of events known to be detrimental to the health condition of the treated fish, or by statistical assessment considering a distinct observation carried out on non-treated fishes, or similar information that can be extrapolated or derived from common knowledge of the skilled person in the art.

“Increase of the lifespan” or “reduction of the mortality” can also be achieved through disease mitigation, alleviation, cure, management or control, at the level of a whole population of fishes. Diseases are discussed above.

According to a specific embodiment, the invention aims at mitigating rainbow trout fish diseases, especially columnaris disease, in the context of aquaculture research or husbandry.

According to a specific embodiment, the bacterial strains or combination thereof is (are) administered to a teleostean fish or a population encompassing a teleostean fish, more particularly a rainbow trout or a population encompassing a rainbow trout or, in aquaculture settings or fish husbandry settings. Apart trouts, a list of fishes is provided in the Table above, which is referred to in the present paragraph. Particular fishes are indicated as well, above.

Aquaculture settings may be natural settings or tanks, in fresh water or sea water, in flow-through or recirculation water feed management.

According to a specific embodiment, the bacterial strains or combination thereof is(are) used for preventing or mitigating a rainbow trout fish disease in aquaculture settings or fish husbandry settings while a pathogen, especially a pathogen as defined in any embodiment herein, has been detected in the aquaculture settings or fish husbandry settings, especially the rainbow trout fish disease that is columnaris disease.

According to a particular embodiment, bacterial strains or combination thereof, including their variant(s), is(are) introduced into fishes commensal microbial community by adding directly the bacterial strains or combination thereof into the fish raising water, in form of live bacteria.

Possible administration routes, and modes of administration to hosts to be treated, can be deduced of the different embodiments and/or formulations of the active ingredient(s) to be delivered, detailed in the present description, here above:
Active ingredient(s) of the present invention can be delivered in individualized form or within mixtures of active ingredients, with extra components or not. Active ingredient(s) may be encapsulated, and/or
Active ingredient(s) can be administered as, non limitatively recited, capsules, liquids, powder, powder beads, tablets . . . and/or
Active ingredient(s) may be administered, in any form or type of formulation as described herein, along with meals (such as with so-called fish meal, and/or fish oil), or when contained within food products. Accordingly, instant invention also concerns a kit comprising or consisting essentially of, or consisting of active ingredient(s) of instant invention, according to any embodiment recited herein, and at least one food product, in particular a fish food product, and if appropriate a leaflet for administration instructions with a view of achieving the effect(s) described herein.

Accordingly, active ingredient(s) of the invention can be administered directly into the hosts' environment, for example when added to the water where fishes, including larvae, live and grow, or be directly administered to the hosts' body, by any route, especially routes involving, i.e., allowing the bacterial strain(s) to be put into contact with, the gastro-intestinal tract (GIT) of the fish (liquid or food).

The invention is generally described herein using the wording “for use” when applications are contemplated. Using a synonymous wording, the invention also relates to method(s) of administering to a host in need thereof, in particular a fish, probiotic active ingredient(s) as defined in any of the embodiments described herein, including variants. All instances of “for use” can therefore we reworded as method(s) of administering the active ingredients including variants described herein to a host, for the purpose(s) described herein, according to any described embodiment.

The invention also relates to method(s) for:
a. preventing the occurrence of a further infection by a Flavobacterium columnare pathogen, or minimizing the consequences of an ongoing infection by a Flavobacterium columnare pathogen, and/or
b. enhancing the resistance against a Flavobacterium columnare pathogen, and/or
c. preventing or minimizing the extent (spread and consequences) of subsequent infections by a Flavobacterium columnare pathogen, or enhancing the resistance against a Flavobacterium columnare pathogen, and/or
d. preventing or controlling diseases, notably caused by an infection by the Flavobacterium columnare pathogen, especially preventing or minimizing disease propagation within the treated population of hosts species, and/or
e. increasing the lifespan of treated host(s) species or population of treated hosts species), or reducing the mortality of treated host(s) species or population of treated hosts species, which is(are) preferably fish(es).

In host(s) species or treated population of hosts species, which is(are) preferably fish(es), administered with active ingredient(s) of instant invention, according to any embodiment described herein for administration to the host(s).

The invention also relates to a bacterial strain selected from the group consisting of: a Chryseobacterium massiliae strain identified by Accession Number No. I-5479 deposited at the CNCM on Jan. 24, 2020 (SEQ ID NO: 2) and a Flavobacterium sp. strain identified by Accession Number No. I-5481 deposited at the CNCM on Jan. 24, 2020 (SEQ ID NO:1), or variants thereof wherein one or more virulence factor coding gene(s) and/or antibiotic resistance genes is(are) deleted or inactivated.

When full or lengthy genome sequences are considered, especially by way of reference to the SEQ ID NOs of the present disclosure, but also by way of reference to sequences that can be obtained by sequencing the genome of deposited strains, as described herein, it can be appreciated that the claimed invention shall not be affected by errors arising from the sequencing process. Examples of protocols and sequencing methods are provided herein and are well known from the skilled person, who can readily determine sequencing error(s) according to common knowledge.

According to a particular embodiment, the bacterial strain is an isolated bacterial strain.

According to a particular embodiment, the bacterial strain is an isolated and live bacteria. According to a particular embodiment, the bacterial strain is an isolated and lyophilized bacterial strain, which however keeps the property of being rehydrated and/or revived and/or revitalized, when relevant.

According to another aspect, the invention also encompass:
(i) a Chryseobacterium massiliae strain with at least 95% or more Average Nucleotide Identity (ANI) value with the Chryseobacterium massiliae strain identified by Accession Number No. I-5479 deposited at the CNCM on Jan. 24, 2020 (also disclosed under SEQ ID NO:2), but differing from the later by at least one (i.e., one, two, three or more) nucleotide deletion or substitution with respect to the natural deposited strain, especially through nucleotide deletion or substitution achieved by non-natural sequence engineering, so that the resulting strain does not have any natural counterpart, especially any genetically identical counterpart, but keeps the property(ies) detailed herein with respect to strain No. I-5479,
(ii) a Flavobacterium. sp. Strain, with at least 95% or more Average Nucleotide Identity (ANI) value with the Flavobacterium sp. strain identified by Accession Number No. I-5481 deposited at the CNCM on Jan. 24, 2020 (also disclosed under SEQ ID NO: 1), but differing from the later by at least one (i.e., one, two, three or more) nucleotide deletion or substitution with respect to the natural deposited strain, especially through nucleotide deletion or substitution achieved by non-natural sequence engineering, so that the resulting strain does not have any natural counterpart, especially any genetically identical counterpart, but keeps the property(ies) detailed herein with respect to strain No. I-5481,
(iii) or variants of a Chryseobacterium massiliae strain with at least 95% or more Average Nucleotide Identity (ANI) value with the Chryseobacterium massiliae strain identified by Accession Number No. I-5479 deposited at the CNCM on Jan. 24, 2020 (also disclosed under SEQ ID NO:2), or a variant of a Flavobacterium. sp. Strain, with at least 95% or more Average Nucleotide Identity (ANI) value with the Flavobacterium sp. strain identified by Accession Number No. I-5481 deposited at the CNCM on Jan. 24, 2020 (also disclosed under SEQ ID NO: 1), or a variant of any one of the above strains of (i) or (ii), according to all described embodiments, wherein one or more virulence factor coding gene(s) and/or antibiotic resistance genes is(are) deleted or inactivated.

It is to be understood that instant invention also encompass a strain modified, engineered so as to differ from any naturally existing strain, although being structurally close from the naturally identified strain, and keeping the property(ies) detailed herein with respect to the deposited strains described herein by reference to Accession Numbers at the CNCM, or by reference to full genome sequences. Variants described herein fall within this embodiment.

The invention conversely also concerns a food product, especially a fish food product, solid or liquid, comprising bacterial strain(s) of the invention, as described in any embodiment herein including variants, or mixture thereof.

The invention also concerns encapsulated bacterial strain(s) of the invention, as described in any embodiment herein including variants, or mixture thereof.

The invention also concerns the use of bacterial strain(s) of the invention, as described in any embodiment herein including variants, or mixture thereof, or encapsulated bacterial strain(s) of the invention, as described in any embodiment herein including variants, or mixture thereof, or compositions of the invention as described herein, as additives (to food products), especially probiotic additives to food products. Food products may be dry (solid) or liquid products.

In a particular embodiment, bacterial strain(s) of the invention including variants is(are) administered as and when found in a food-based product, or is(are) administered in association with a food-based product. Carrier or delivery vehicle(s) as mentioned herein are adapted accordingly, following the conventional practice in the field. When in a food product or administered in association with a food product, bacterial strain(s) may be encapsulated.

Dosage can be readily determined by the skilled person, as conventionally done in the field.

According to a particular embodiment, bacterial strains or combination thereof including variants is(are) administered to a host in need thereof in a dosage or in a formulation enabling a final liquid dosage ranging from 5×10⁴ cfu/mL to 5×10⁶, or up to 5×10⁸ cfu/mL of active ingredient(s), in particular a dosage of 5×10⁵ cfu/mL. The invention extends to a dry/solid dosage enabling a liquid dosage equivalent to the dosage described above. Dosage applied to dry forms may be expressed in grams instead of mL. This range and particular values are susceptible of conferring a total protection to the host, especially a fish host, against subsequent infections by Flavobacterium columnare pathogen. In this context, the level of protection achieved can be evidenced by in vivo infection challenges using gnotobiotic zebrafish or rainbow trout as an animal model, examples of which are described herein or in the literature. Examples described herein show that whereas F. columnare killed Germ Free (GF) fish larvae in less than 48 h, fish exposed to Chryseobacterium massiliae or Flavobacterium sp. strains of the invention were more resistant to the infection, with an increase in survival up to 90% by reference to experiment where no protective bacterial strain(s) of the invention were administered to the model. Accordingly, it can be defined that “total” protection can be assessed by an in vivo infection challenge test using gnotobiotic zebrafish or rainbow trout as an animal model, where an increase in survival of the model up to 90% can be observed after administration of the considered strain(s) of the invention to the model. Guidance can be found in the experimental section regarding the implementation of this test.

Of note, such a dosage enabling a “total” protection requires concentration of the active ingredient(s), which is mark of technical engineering brought to the administered product.

The invention also relates to a probiotic composition comprising or consisting essentially of, or consisting of, at least one bacterial strain selected from the group consisting of: a Chryseobacterium massiliae strain and a Flavobacterium. sp. strain with at least 95% or more Average Nucleotide Identity (ANI) value with the Flavobacterium sp. strain identified by Accession Number No. I-5481 deposited at the CNCM on Jan. 24, 2020, according to any combination thereof, in particular at least two or three distinct bacterial strains from said group, and further acceptable carrier or delivery vehicle(s) and optionally adjuvant component(s).

The bacterial strain that can be found in compositions of the invention, are the same as those described in the present description, especially above, in the context of their intended administration, notably encompassing all described variations and embodiments.

In particular probiotic compositions of the invention can encompass variants of naturally existing strain(s), as defined herein especially with respect to modified strains, engineered so as to differ from any naturally existing strain, and keeping the property(ies) detailed herein with respect to the deposited strain(s) described herein by reference to Accession Number(s) at the CNCM.

Probiotic compositions of the invention also encompass compositions comprising or consisting essentially of or consisting of any strain as described herein or mixture thereof, at a dosage enabling “total” protection of the host as defined herein.

By acceptable carrier or delivery vehicle(s), it is meant any substance, agent, ingredient, that can be safely administered to a host for the purpose of delivering the active ingredients to said host so that they can achieve their function, i.e, their probiotic function. They may be water or another other liquid, or other acceptable substance, agent, ingredient. They may be pharmaceutical or veterinary carrier or delivery vehicle(s). They may be food. Details regarding acceptable carrier or delivery vehicle(s) can be found above in the present description, these sections are referred to also in the context of composition(s) as described herein. When food is used as a vehicle for probiotic bacteria or compositions of the invention, it can enable that the probiotics will arrive to the fish gut.

Is also described a probiotic composition comprising a Flavobacterium sp. strain of the invention (characterized by reference to a deposited strain—see present description), and further comprising the following bacterium: the Delftia acidovorans strain identified by Accession Number No. I-5480 deposited at the CNCM on Jan. 24, 2020 ((sequence available in ENA (European Nucleotide Archive) database under primary accession number ERS4574863 (version 1) and secondary accession). Aeromonas rivipollensis, Pseudomonas helmanticensis, Aeromonas rivipollensis, Pseudomonas baetica, Aeromonas hydrophyla, Flavobacterium plurextorum, Acinetobacter sp., Flavobacterium plurextorum, Pseudomonas sp.

In this context, said bacterium may be isolated from the microbiota of a trout, in particular a rainbow trout.

According to another aspect described herein, a composition comprises a Chryseobacterium massiliae strain and further comprises the following bacterium: at least one Aeromonas veronii strain, in particular two distinct Aeromonas veronii strain, Pseudomonas mossellii, Stenotrophomas maltophilia, Aeromonas caviae, Pseudomonas peli, Pseudomonas sediminis, Phyllobacterium myrsinacearum, Pseudomonas nitroreducens.

In this context, said bacterium may be isolated from the microbiota of a zebrafish.

According to a particular embodiment, at least one bacterial strain of a probiotic composition of the invention, is from the group consisting of: a Chryseobacterium massiliae strain identified by Accession Number No. I-5479 deposited at the CNCM on Jan. 24, 2020, and a Flavobacterium sp. strain identified by Accession Number No. I-5481 deposited at the CNCM on Jan. 24, 2020, and variants thereof wherein one or more virulence factor coding gene(s) and/or antibiotic resistance genes is(are) deleted or inactivated.

According to a particular embodiment, at least one bacterial strain of a probiotic composition of the invention, is from the group consisting of: a Chryseobacterium massiliae strain comprising or consisting essentially of, or consisting of the genome of SEQ ID NO:2, and a Flavobacterium sp. strain comprising or consisting essentially of or consisting of the geneome of SEQ ID NO:1, and variants thereof wherein one or more virulence factor coding gene(s) and/or antibiotic resistance genes is(are) deleted or inactivated.

Those probiotic composition of the invention as described in any one of the embodiments described herein, in particular in the preceding paragraphs concerning probiotic compositions, can be used for the purpose detailed in any embodiment described in the present description.

The invention also relates to a method of manufacturing a composition, formulation, food product (liquid or dry), or kit containing the active ingredient(s) described herein, according to any embodiment herein disclosed.

The invention also relates to a method of using any of the active ingredient(s) described herein, according to any embodiment herein disclosed including variants, for producing or manufacturing a composition, formulation, food product (liquid or dry), or kit suitable for being administered to a host, in particular a fish, in need thereof. The composition, formulation, food product (liquid or dry), or kit may be used for probiotic or prophylactic purpose, or against a disease in the host, according to any embodiment described herein.

The invention also relates to the use any of the active ingredient(s) described herein, according to any embodiment herein disclosed including variants, for producing or manufacturing a composition, formulation, food product (liquid or dry), or kit suitable for being administered to a host, in particular a fish, in need thereof.

The invention also relates to the use any of the active ingredient(s) described herein, according to any embodiment herein disclosed including variants, for administration to a host, in particular a fish, in need thereof. Administration may be intended for probiotic or prophylactic purpose, or against a disease in the host, according to any embodiment described herein.

The invention also relates to a method to identify bacterial strain(s) that are probiotic against a pathogen infection, comprising the steps of:
a) Providing a germ-free trout model that is susceptible of being infected, with adverse effect on its health condition, by at least one determined pathogen, the latter of which does not infect a conventional trout with adverse effect on its health condition,
b) Determining the microbiota, especially the culturable microbiota, of the said conventional trout of step a) and optionally identifying the most represented bacterial strains of said microbiota, further optionally after isolating the said most represented bacterial strains of said microbiota (so that identification is possible).
c) Exposing the germ-free trout model to the at least one determined pathogen of step a) after reconventionalization of the germ-free trout model through inoculation of the germ-free trout with either the microbiota determined in b) or with the most represented bacterial strain(s) of said microbiota identified in b), when identified,
d) Concluding about the probiotic effect of the microbiota or the most represented bacterial strains thereof, inoculated to the reconventionalized germ-free trout model.

With regards to step a), the germ-free trout model may consist in trout larvae. According to a particular embodiment, the germ-free trout model is a rainbow trout. Present description details how inventors obtained such a new model, for the first time.

According to a more particular embodiment, step a) may alternatively encompass identifying a pathogen that infect, in particular kills, a germ-free trout model, but does not infect, in particular kills, a conventional trout.

The fact that the provided pathogen kills the used germ-free trout model is a possibility to increase the level of stringency of the above-recited method to identify bacterial strain(s). Indeed, with such a criteria the method provides more relevant hits (i.e., can be termed a “high throughput” method). Nevertheless, the skilled person can appreciate that observation of symptoms of lesser gravity than the death of the fish does not prevent the method of being successfully carried out (i.e. by observing symptoms of infection in the fish and their possible alleviation in step c)), albeit in a less efficient way (lower throughput, but hits still identified).

Particular examples detailing how steps a) to d) can be achieved are thoroughly exemplified in the experimental section of the present description. It will be understood that the method to identify bacterial strain(s) described herein can be applied to a great number of distinct pathogens (in step a)), and a large number of distinct microbiota (which may partly depend upon the environment), depending on the circumstances of the experiments. The skilled person can readily determine the circumstances of the experiments, guidance is provided in the experimental section hereafter.

By “reconventionalization”, it meant inoculating a microbiota of relevance to the host who lacks said microbiota. Comprehensive examples, and guidance that can be derived therefrom, are provided herein.

Conclusion in step d) can be made by observing the biological effect obtained after the challenge of step c), on the reconventionalized trout model. Comprehensive examples, and guidance that can be derived therefrom, are provided herein.

The term “comprising” as used herein, which is synonymous with “including” or “containing”, is open-ended, and does not exclude additional, unrecited element(s), ingredient(s) or method step(s), whereas the term “consisting of” is a closed term, which excludes any additional element, step, or ingredient which is not explicitly recited.

The term “essentially consisting of” is a partially open term, which does not exclude additional, unrecited element(s), step(s), or ingredient(s), as long as these additional element(s), step(s) or ingredient(s) do not materially affect the basic and novel properties of the application.

The term “comprising” (or “comprise(s)”) hence includes the term “consisting of” (“consist(s) of”), as well as the term “essentially consisting of” (“essentially consist(s) of”). Accordingly, the term “comprising” (or “comprise(s)”) is, in the present application, meant as more particularly encompassing the term “consisting of” (“consist(s) of”), and the term “essentially consisting of” (“essentially consist(s) of”).

In an attempt to help the reader of the present application, the description has been separated in various paragraphs or sections. These separations should not be considered as disconnecting the substance of a paragraph or section from the substance of another paragraph or section. To the contrary, the present description encompasses all the combinations of the various sections, paragraphs and sentences that can be contemplated.

Each of the relevant disclosures of all references cited herein is specifically incorporated by reference.

The features described here-above and other features of the invention will be apparent when reading the examples and the figures, which illustrate the experiments conducted by the inventors, in complement to the features and definitions given in the present description. The following examples are offered by way of illustration. The examples are however not limitative with respect to the described invention.

### LEGEND OF THE FIGURES

FIG. 1. Flavobacterium columnare kills germ-free but not conventional zebrafish. 6 dpf GF or Conv zebrafish larvae were exposed to different doses of F. columnare^{ALG} by bath immersion and transferred after 3 h into sterile water. Mean survival is represented by a thick horizontal bar with standard deviation. For each condition, n=12 zebrafish larvae. Larvae mortality rate was monitored daily and surviving fish were euthanized at day 10 post infection. Statistics correspond to unpaired, non-parametric Mann-Whitney test comparing all conditions to non-infected GF (left) or Conv (right). ****: p<0.0001; absence of *: non-significant. Blue (grey in black and white version) mean bars correspond to non-exposed larvae and red (light grey in black and white version)mean bars correspond to larvae exposed to F. columnare.

FIG. 2. Screen for F. columnare strains killing germ-free zebrafish. A. Survival of GF zebrafish larvae exposed to a collection of 28 strains of F. columnare. B. Survival of GF and Conv zebrafish larvae exposed to the 7 most virulent F. columnare strains. Zebrafish larvae were infected at 6 dpf by immersion for 3 h with 5.10² cfu/mL. Mean survival is represented by a thick horizontal bar with standard deviation. For each condition, n=12 zebrafish larvae. Larvae mortality rate was monitored daily and surviving fish were euthanized at day 10 post infection. Statistics correspond to unpaired, non-parametric Mann-Whitney test comparing all conditions to non-infected GF. ****: p<0.0001; ***: p<0.001: *: p<0.005 *: p<0.05; No star: non-significant. Blue/grey mean bars correspond to non-infected larvae and red (light grey in black and white version) mean bars correspond to infected larvae.

FIG. 3. Conventional larvae from 4 different zebrafish facilities are also protected against F. columnare infection. Zebrafish AB line eggs from 4 different zebrafish facilities were collected: Facility 1—Hopital Robert Debré academic facility, Paris, France; Facility 2 and 3—two academic facilities at University Paris 6, Paris, France; Facility 4—commercial Amagen facility in Gif-sur-Yvette, France. 6 dpf GF or Conv zebrafish larvae from each facility were exposed to F. columnare^{ALG} by bath immersion and transferred after 3 hours to sterile water. . Mean survival is represented by a thick horizontal bar with standard deviation. For each condition, n=12 zebrafish larvae. Blue (grey in black and white version) mean bars correspond to non-infected larvae and red (light grey in black and white version) mean bars correspond to infected larvae. Larvae mortality rate was monitored daily and surviving fish were euthanized at day 10 post infection. Indicated statistics correspond to unpaired, non-parametric Mann-Whitney test. ****: p<0.0001; absence of *: non-significant.

FIG. 4. Reconventionalization of germ-free zebrafish larvae confers protection against F. columnare infection.

Resistance to F. columnare^{ALG} of GF and Conv zebrafish larvae placed in contact with fish facility tank water or mashed non-sterile eggs at 0 (sterilization day) or 4 dpf (hatching day). F. columnare^{ALG} inoculum doses=5×10⁵ cfu/mL. Mean survival is represented by a thick horizontal bar with standard deviation. For each condition, n=12 zebrafish larvae. Larvae mortality rate was monitored daily and surviving fish were euthanized at day 10 post infection. Statistics correspond to unpaired, non-parametric Mann-Whitney test comparing all conditions to non-infected non-infected GF ****: p<0.0001; absence of *: non-significant. Blue (grey in black and white version) mean bars correspond to non-infected larvae and red (light grey in black and white version) mean bars correspond to infected larvae.

FIG. 5. A mix of the 10 culturable bacteria constituting the core zebrafish larvae microbiota confers protection against F. columnare infection. The 10 culturable strains identified as the core conventional microbiota were added as an equivalent concentration of 5×10⁵ cfu/mL mix to 4 dpf larvae followed by infection challenge at 6 dpf. Mean survival is represented by a thick horizontal bar with standard deviation. For each condition, n=12 zebrafish larvae. Larvae mortality rate was monitored daily and surviving fish were euthanized at day 10 post infection. Indicated statistics correspond to unpaired, non-parametric Mann-Whitney test. ****: p<0.0001; absence of *: non-significant. Blue (grey in black and white version) mean bars correspond to non-infected larvae and red (light grey in black and white version) mean bars correspond to infected larvae.

FIG. 6. Dynamics of bacterial community establishment in reconventionalized zebrafish larvae. Relative abundances of the 10 species composing the identified Mix10 core protective zebrafish microbiota at different time points using 16S rDNA gene amplicon sequencing of pools of 10 larvae. Left bar represents the relative abundance of microbiota species of conventional zebrafish larvae on hatching day (4 dpf). For Re-Conv Mix10 populations, Tetrahymena-fed GF larvae were incubated with an equiratio combination of the 10 species (5×10⁵ cfu/mL each) composing the microbiota core at 4 dpf.

FIG. 7. Effect of antibiotic treatment on zebrafish survival. GF and Conv zebrafish larvae were exposed to antibiotic treatment for 16 hours at 4 dpf. Antibiotics were then washed off and the zebrafish larvae were infected with F. columnare^{ALG}. Different concentrations of penicillin/streptomycin or kanamycin were used to identify a non-toxic antibiotic treatment causing microbiota dysbiosis. Penicillin/streptomycin dose 1=250 μg/mL; dose 2=15,6 μg/mL; kanamycin dose 1=200 μg/mL; dose 2=50 μg/mL; dose 3=25 μg/mL. Mean survival is represented by a thick horizontal bar with standard deviation. For each condition, n=12 zebrafish larvae. Blue (grey in black and white version) mean bars correspond to non-infected larvae and red (light grey in black and white version) mean bars correspond to infected larvae. Larvae mortality rate was monitored daily and surviving fish were euthanized at day 10 post infection. Indicated statistics correspond to unpaired, non-parametric Mann-Whitney test. ****: p<0.0001; ***: p<0.001; **: p<0.005 *: p<0.05; absence of *: non-significant.

FIG. 8. Analysis of protection against F. columnare infection after antibiotic dysbiosis. A: Response of zebrafish larvae to F. columnare^{ALG} infection after antibiotic-induced dysbiosis with a diagram showing timing and treatments of the experiment. B: A 24 h period after antibiotic treatment allows the recovery of protection in kanamycin-treated zebrafish larvae with a diagram showing timing and treatments. Mean survival is represented by a thick horizontal bar with standard deviation. For each condition, n=12 zebrafish larvae. Blue (grey in black and white version) mean bars correspond to non-infected larvae and red (light grey in black and white version) mean bars correspond to infected larvae. Larvae mortality rate was monitored daily and surviving fish were euthanized at day 10 post infection. Indicated statistics correspond to unpaired, non-parametric Mann-Whitney test. ****: p<0.0001; absence of *: non-significant. C: Community recovery profile with streptomycin/penicillin treatment. D: Community recovery profile with kanamycin treatment. Pools of 10 larvae were collected for 16S rDNA sequencing.

FIG. 9. Protection of zebrafish against F. columnare reconventionalized with individual or mixed bacterial strains isolated from zebrafish larvae. A: Determination of the level of protection provided by each of the 10 bacterial species composing the core protective zebrafish microbiota. Bacteria were added individually to the water on hatching day (dose 5×10⁵ cfu/ml). B: Level of protection provided by different amount of C. massiliae and Mix9. Mix9 only protected at the highest inoculum doses. Mean survival is represented by a thick horizontal bar with standard deviation. For each condition, n=12 zebrafish larvae. Blue (grey in black and white version) mean bars correspond to non-infected larvae and red (light grey in black and white version) mean bars correspond to infected larvae. Larvae mortality rate was monitored daily and surviving fish were euthanized at day 10 post infection. Indicated statistics correspond to unpaired, non-parametric Mann-Whitney test. ****: p<0.0001; absence of *: non-significant.

FIG. 10. Combinations of 8 species from the protective Mix9 do not protect against F. columnare infections. A: Zebrafish larvae survival to F. columnare^{ALG} infection at 6 dpf after reconventionalization with different possible mixes including 8 species of the Mix9 consortium. Mix10=Re-ConvMix¹⁰. Mix9=Re-Conv^{Mix9}, Mix8=Re-Conv^{Mix8}. B: table showing different combinations used for the reconventionalization at 4 dpf. C: Zebrafish larvae survival to F. columnare^{ALG} infection at 6 dpf after reconventionalization with eight different combinations of the non-protective Mix8a were tested, in which the quantity of 1 of the 8 species was doubled in each combination (indicated in yellow in D). D: table showing different combinations used for the reconventionalization at 4 dpf. Mix10=Re-ConvMix¹⁰. Mix9=Re-Conv^{Mix9}. Mix8=Re-Conv^{Mix8}. A and C: Mean survival is represented by a thick horizontal bar with standard deviation. For each condition, n=12 zebrafish larvae. Blue (grey in black and white version) mean bars correspond to non-infected larvae and red (light grey in black and white version) mean bars correspond to infected larvae. Larvae mortality rate was monitored daily and surviving fish were euthanized at day 10 post infection. Indicated statistics correspond to unpaired, non-parametric Mann-Whitney test. ****: p<0.0001; absence of *: non-significant.

FIG. 11. Zebrafish immune response to F.columnare infection. A-C: qRT-PCR analysis of host gene expression, 18 hours after exposure to F. columnare, in larvae reconventionalized with indicated bacteria or bacterial mixes: each point corresponds to an individual larva. Expression of il10 (A), il1b (B), and il22 (C), by wild-type AB zebrafish; D: Comparison of the survival of myd88−/− and background-matched myd88+/+ zebrafish after reconventionalyzation and exposure to F. columnare^{ALG}. Mean survival is represented by a thick horizontal bar with standard deviation. For each condition, n=12 zebrafish larvae. Larvae mortality rate was monitored daily and surviving fish were euthanized at day 10 post infection. A-D: Blue (grey in black and white version) bars correspond to non-infected larvae and red (light grey in black and white version) bars correspond to infected zebrafish. Indicated statistics correspond to unpaired, non-parametric Mann-Whitney test. ****: p<0.0001; ***: p<0.001; **: p<0.005 *: p<0.05; absence of *: non-significant.

FIG. 12. Expression of il1b in WT and myd88^{-/-} zebrafish mutants reconventionalized with indicated various bacteria or bacterial mixes, qRT-PCR analysis of il1b gene expression were performed 18 hours after exposure to F. columnare^{ALG}. Each point corresponds to an individual larva. Blue (grey in black and white version) mean bars correspond to non-infected larvae and red (light grey in black and white version) mean bars correspond to infected larvae. Larvae mortality rate was monitored daily and surviving fish were euthanized at day 10 post infection. Indicated statistics correspond to unpaired, non-parametric Mann-Whitney test. ****. p<0.0001; *: p<0.001; *: p<0.005 *: p<0.05; absence of *: non-significant.

Mean survival is represented by a thick horizontal bar. Blue (grey in black and white version) bars correspond to non-infected larvae and red (light grey in black and white version) bars correspond to infected zebrafish.

FIG. 13. Intestine of infected germ-free zebrafish displays severe disorganization. Germ-free, conventional and reconventionalized zebrafish larvae. Reconventionalized zebrafish were inoculated at 4 dpf with Mix9 or C. massiliae. A: Representative picture of intestines of non-infected larvae. Fish were fixed for histology analysis or electron microscopy at 7 dpf. Left: Giemsa coloration L=intestinal lumen Right. Transmission electron microscopy. B: Representative picture of intestines of infected larvae exposed at 7 dpf to F. columnare^{ALG}. Toluidine blue staining of Epon-embedded zebrafish larvae for Light microscopy (left) or Transmission electron microscopy at 7 dpf (right).

FIG. 14. F. columnare infection larvae requires food ingestion. GF zebrafish larvae were fed with sterile T. thermophila (GF+Tetra) or sterile fish food powder (GF+powder) or were not fed before F.columnare^{ALG} infection. Whereas, fed larvae were sensitive to F.columnare^{ALG} infection, un-fed GF larvae did not die after fish pathogen infection. Mean survival is represented by a thick horizontal bar with standard deviation. For each condition, n=12 zebrafish larvae. Blue (grey in black and white version) mean bars correspond to non-infected larvae and red (light grey in black and white version) mean bars correspond to infected larvae. Larvae mortality rate was monitored daily and surviving fish were euthanized at day 10 post infection. Indicated statistics correspond to unpaired, non-parametric Mann-Whitney test. ****: p<0.0001; ***: p<0.001: *: p<0.005 *: p<0.05; absence of *: non-significant.

FIG. 15. Pre-exposure to C. massiliae protects larval and adult zebrafish against F. columnare infection

A: Zebrafish larvae were inoculated at 4 dpf with 5×10⁵ cfu/ml of C. massiliae for 48 h before infection at 6 dpf with virulent F. columnare strains. B: Survival of adult zebrafish with or without pre-exposure to C. massiliae (2×10⁶ cfu/mL for 48 h) followed by exposure to F. columnare^{ALG} (5×10⁶ cfu/mL for 1 h) Mean survival is represented by a thick horizontal bar with standard deviation. For each condition, n=12 zebrafish larvae or adult. Zebrafish mortality rate was monitored daily and surviving fish were euthanized at day 10 post infection. Blue (grey in black and white version) bars correspond to non-infected larvae and red (light grey in black and white version) bars correspond to infected zebrafish. Indicated statistics correspond to unpaired, non-parametric Mann-Whitney test. ****: p<0.0001; **: p<0.005 *; absence of *: non-significant.

FIG. 16. Protocol used in this study to raise and infect or re-conventionalized germ-free trout larvae

FIG. 17. Protocol used in this study to raise and infect or re-conventionalized germ-free trout larvae. After fertilization, eggs are sterilized (−5 dph) and kept in sterile, autoclaved mineral water at 16° C. in Petri dish until hatching. Once hatched, rainbow trout larvae are transferred to vented cap cell culture flasks, where they are kept all the experiment. Larvae are fed every 2 days with sterile powder food after 21 dph until the end of the experience; water was renewed 30 minutes after animals feeding. To test the protective effect of potentially probiotic strains, larvae were re-conventionalized by one or several commensal bacteria diluted in water at 22 dph. Pathogenic bacteria are added to the water at 24 dph for 24 h and then larvae are washed with fresh sterile water.

FIG. 18. Growth performance of rainbow trout larvae raised under germ-free and conventional conditions. Conventional and germ-free fish body size (A) and body weight (B) were measured at 35 dph (n=6).

FIG. 19. Anatomical comparison of conventional and GF rainbow trout larvae. Whole-mount were analyzed by optical projection tomography after fish clearing based on immunolabeling-enabled 3D imaging of solvent-cleared organs procedure (iDISCO+). Lateral views of 21 dph imaged conventional (A, B, C and D) and GF (E, F, G and H) rainbow trout larvae. Brain (A and F), spleen (B and G), gut (C and H), gills (D and I), and head kidney (E and J). Images representative of two different fishes per condition.

FIG. 20. Anatomical comparison of the gut of conventional and GF rainbow trout larvae. Whole-mount were analyzed by optical projection tomography after fish clearing based on immunolabeling-enabled 3D imaging of solvent-cleared organs procedure (iDISCO+). Lateral views of 21 dph imaged conventional (A and B) and GF (C and D) rainbow trout larvae. Mid-gut (A and C), and posterior gut (B and D). Images representative of two different fishes per condition.

FIG. 21. Survival of GF and conventional rainbow trout larvae infected with different pathogens. A) GF larvae survival exposed by bath to F. psychrophilum strain THCO2-90, F. columnare strain Fc7, L. garvieae, V. anguillarum and Y. ruckeri strain JIP 27/88. B) F. columnare strain Fc7 kills germ-free but not conventional rainbow trout. Mean and SD plot representing average day post-infection at which infected fish die. For each condition n=12 larvae. All surviving fish were euthanized at day 10 post-infection. Asterisks indicate significant difference from non-infected population (**** p<0.0001).

FIG. 22. Survival of re-conventionalized trout larvae against F. columnare Fc7 infection. A: GF trout larvae exposed to water used to raise conventional fish at 21 dph show similar survival to F. columnare infection than conventional trout larvae. B) The 11 species identified from conventional fish microbiota were added to rainbow trout larvae at 22 dph, followed by F. columnare infection at 24 dph. This bacterial mixture is able to protect re-conventionalized larvae from infection. For each condition n=12 larvae. All surviving fish were euthanized at day 10 after infection(**** p<0.0001).

FIG. 23. Protection of GF trout larvae against F. columnare infection by individual species isolated from the Conv rainbow trout microbiota.

A: The 11 species isolated from Conv fish microbiota (Table 1) were added individually to rainbow trout larvae at 22 dph, followed by F. columnare Fc7 infection at 24 dph. From the 11 different strains, only Flavobacterium sp. strain 4466 protected re-conventionalized larvae from infection. B: Mix11, Mix10 (mix of all identified strain with the exception of Flavobacterium sp. strain 4466), were added to rainbow trout larvae at 22 dph, followed by F. columnare infection at 24 dph. Mix11 protected re-conventionalized larvae from infection, whereas Mix10 did not. For each condition n=10 larvae. All surviving fish were euthanized at day 10 after infection. C: CFU/mL recovered from dissected intestines from GF fish exposed to F. columnare Fc7, Flavobacterium sp. strain 4466 or both. 24 hours post-infection. (**** p<0.0001).

FIG. 24. Chryseobacterium sp. (C. massiliae) provides full trout protection against F. columnare infection. GF trout larvae survival exposed to Chryseobacterium sp. 48 h before infection with F. columnare strains Fc7, IA-S-4, Ms-Fc-4 and ALG-00-530. F. columnare kills germ-free but not those trout previously reconventionalized with Chryseobacterium sp. Mean and SD plot representing average day post-infection at which infected fish die. For each condition n=12 larvae. All surviving fish were euthanized at day 10. Asterisks indicate significant difference from non-infected population (**** p<0.0001; ** p<0.01).

FIG. 25. Survival of GF and Conv rainbow trout larvae infected with different fish pathogens. Kaplan-Meier graph of GF larvae survival after bath exposure to F. psychrophilum strain THCO2-90, F. columnare strain Fc7, L. garvieae strain JIP 28/99. V. anguillarum strain 1669 and Y. ruckeri strain JIP 27/88. Mean and SD plot representing average survival percentage of fish for 10 days after exposition to different pathogenic microorganisms. For each condition n=10 larvae. All surviving fish were euthanized at day 10 post-infection. Asterisks indicate significant difference from non-infected population (** p<0.01; *** p<0.001; **** p<0.0001).

FIG. 26. Survival of re-conventionalized trout larvae against F. columnare Fc7 infection. Kaplan-Meier graph of GF larvae survival after bath exposure to F. columnare strain Fc7, A: F. columnare strain Fc7 kills GF but not Conv rainbow trout. B: GF trout larvae exposed to water used to raise Conv fish at 22 dph show similar survival rates to F. columnare infection than Conv trout larvae. C: The 11 strains identified from Conv fish microbiota were added to rainbow trout larvae at 22 dph, followed by F. columnare infection at 24 dph. This bacterial mixture is able to protect re-conventionalized larvae from infection. Mean and SD plot representing average survival percentage of fish for 10 days after exposition to different pathogenic microorganisms. For each condition n=10 larvae. All surviving fish were euthanized at day 10 after infection. Asterisks indicate significant difference from non-infected population (**** p<0.0001).

FIG. 27. Histological comparison of the posterior gut of infected and non-infected Conv and GF rainbow trout larvae. A: Representative images of intestines of non-infected GF and Conv trout larvae. B: Representative images of intestines of infected GF and Conv larvae exposed to F. columnare strain Fc7. Fish were fixed for histology analysis at 1 day post-infection (dpi). C: Average Goblet cells number per microvilli of posterior gut. Bars represent means±SD per villi of the same area of posterior gut of 3 fish per condition. Alcian blue and PAS combined staining of paraffin-embedded rainbow trout larvae for light microscopy. Images and quantification data are representative of three different fish per condition.

FIG. 28. Representative images of in vitro growth-inhibition activity of Flavobacterium sp. strain 4466 against different virulent F. columnare strains. A: lack of F. columnare Fc7 growth-inhibition after adding 5 μl of Flavobacterium sp. culture supernatant. B: Halo of F. columnare FC7 growth inhibition surrounding Flavobacterium sp. colony on a F. columnare strain Fc7 overlay. C: Halo of growth inhibition of F. columnare ALG-00-530, IA-S-4, and Ms-Fc-4. The agar overlay technique was performed by spreading F. columnare bacterial suspension on soft-agar solution over TYES agar, and then spotting 5 μl of an overnight culture of Flavobacterium sp. strain 4466. Incubation was performed at 28ºC for 24 h. This experiment was performed in triplicate.

FIG. 29. Phylogenetic tree illustrating the relationship between Flavobacterium sp. strain 4466 and the closest 15 Flavobacterium species based on ANI analysis. The tree was constructed with RAxML (version 8.2.8) by using the 400 most conserved proteins across the proteomes of each strain. Bootstrap support values are indicated in the nodes.

### EXAMPLES

### Results

Flavobacterium columnare Kills Germ-Free but not Conventional Zebrafish

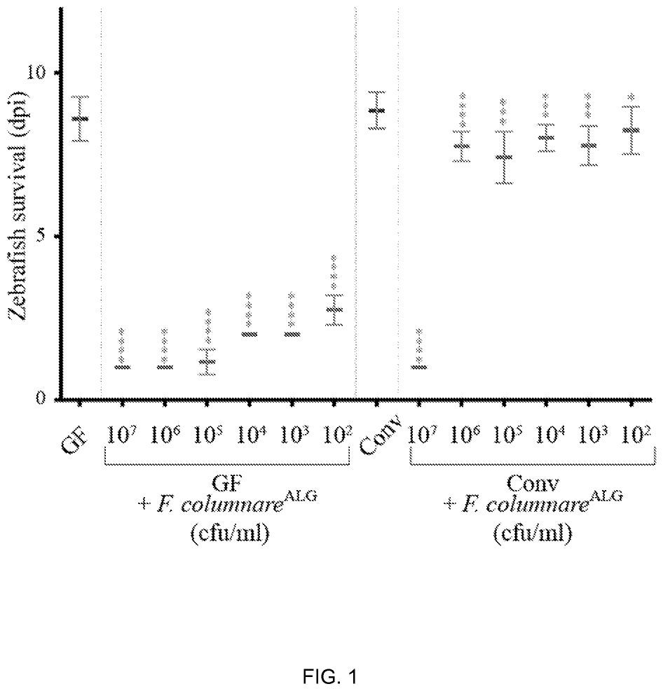

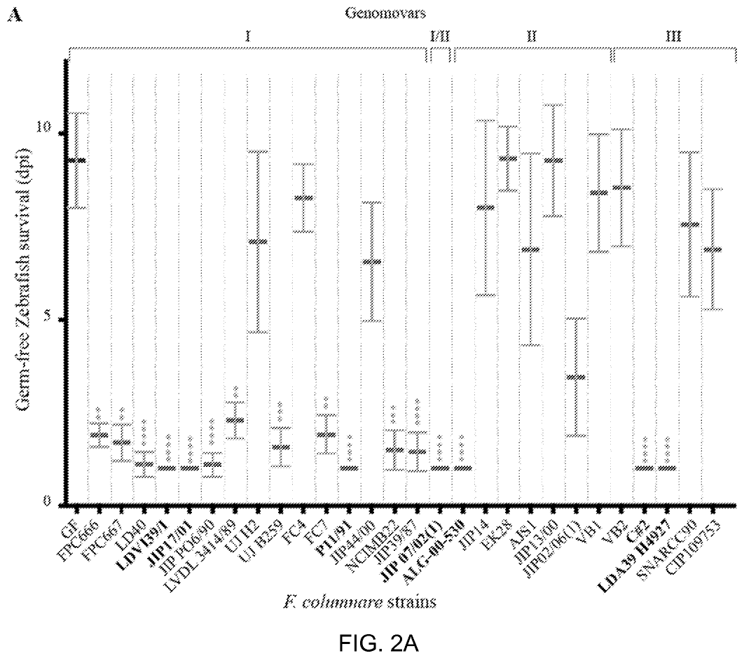

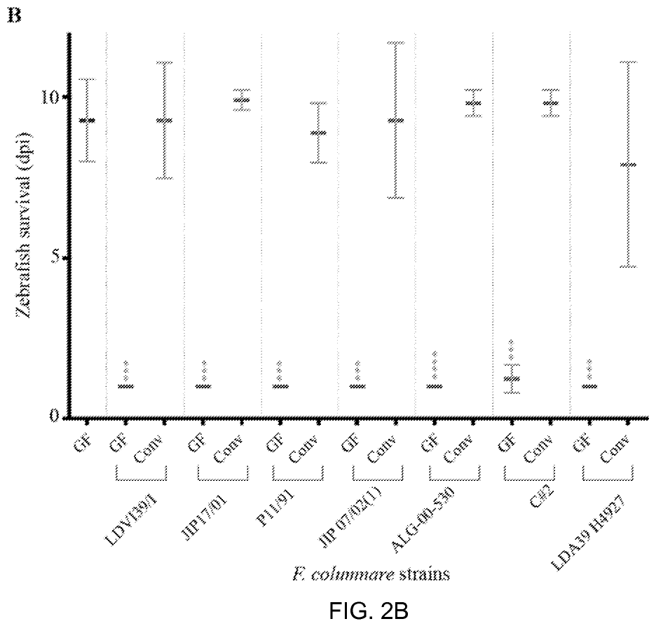

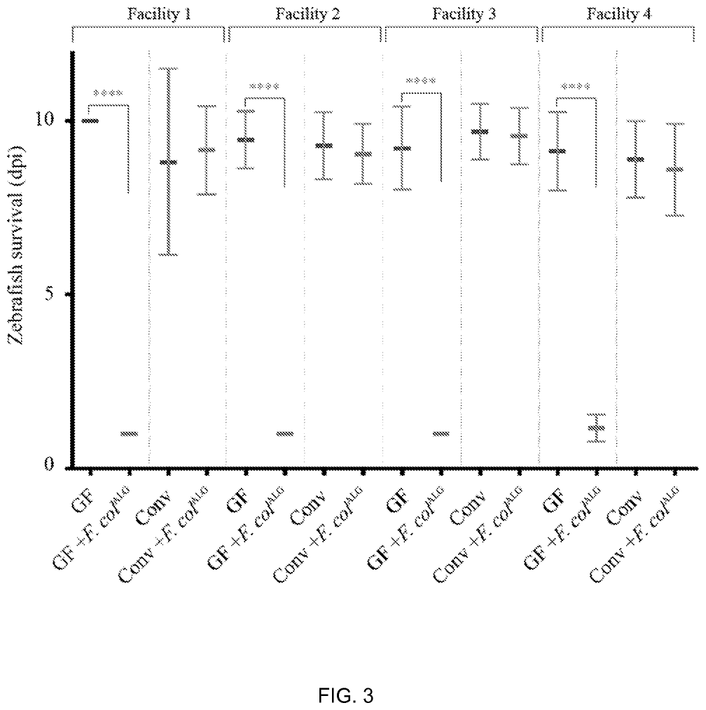

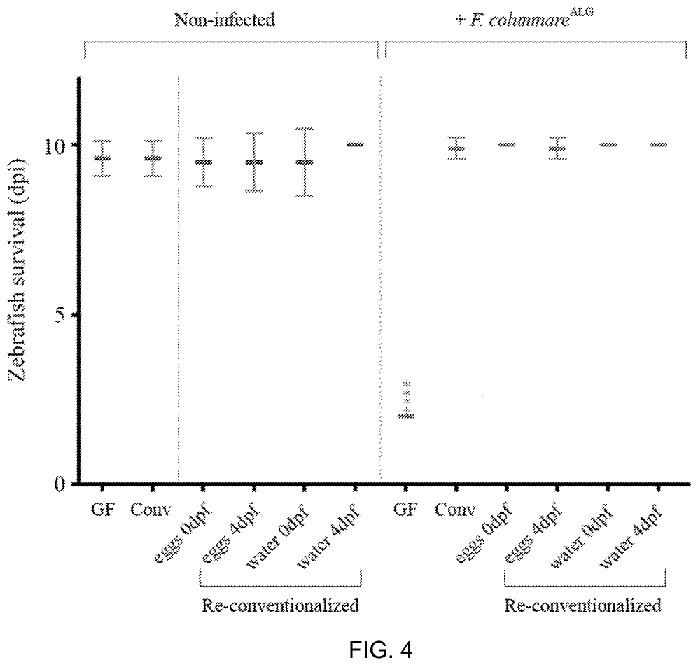

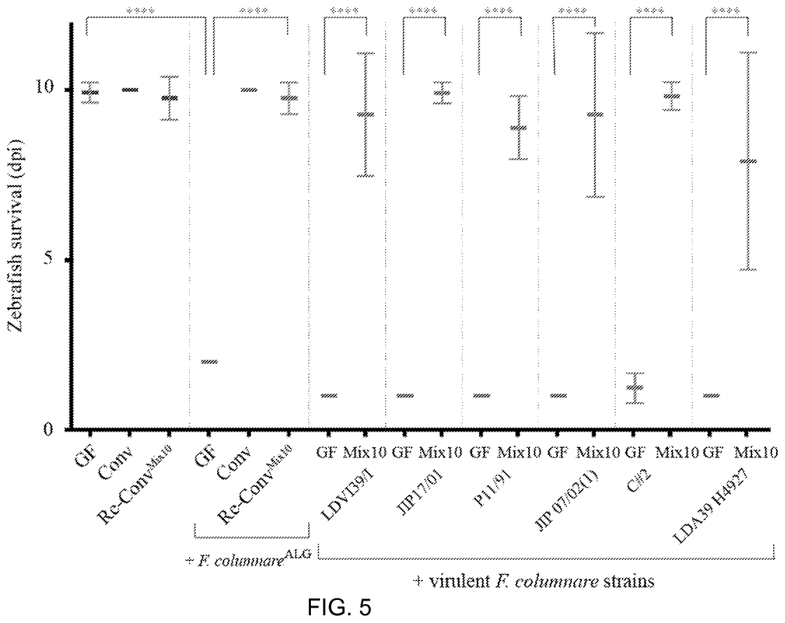

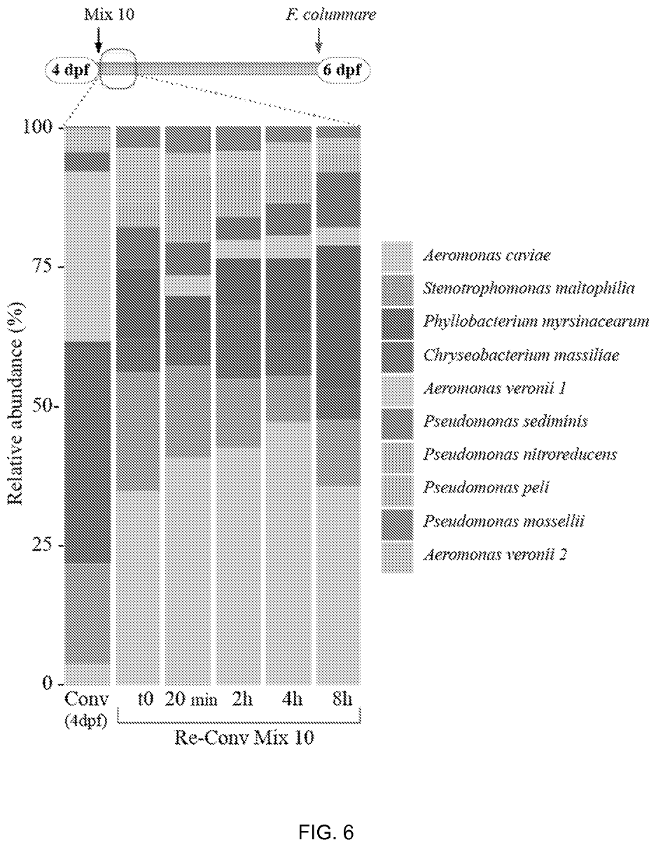

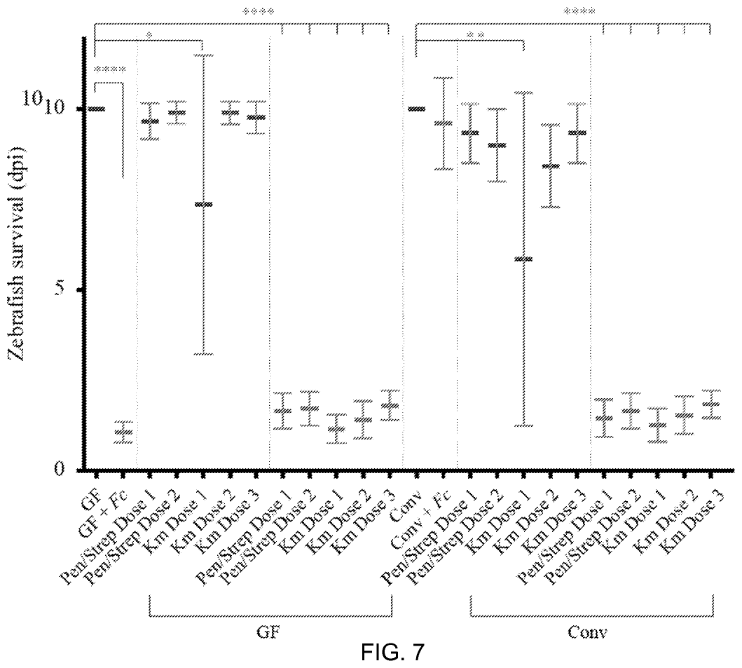

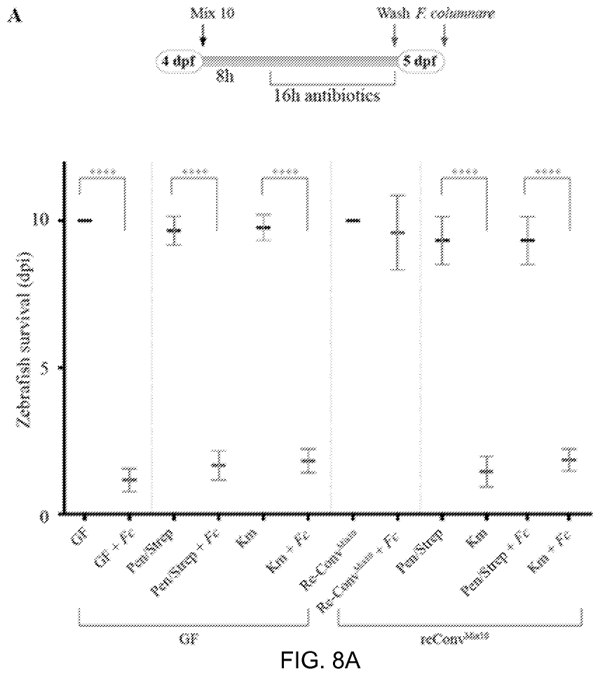

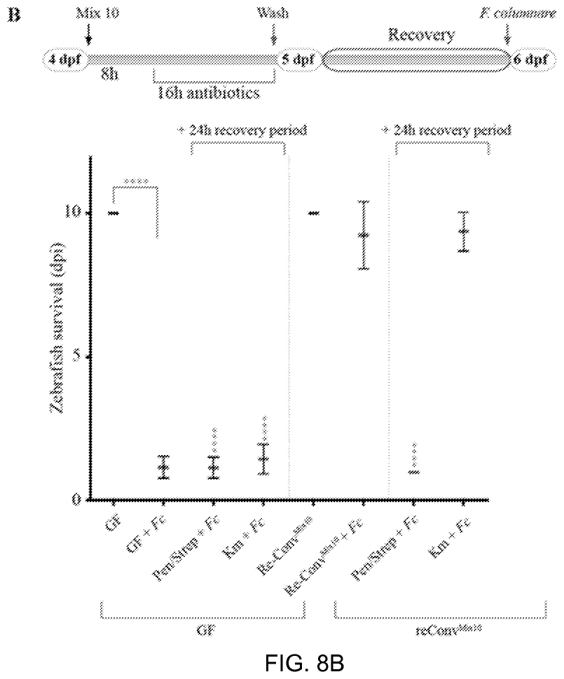

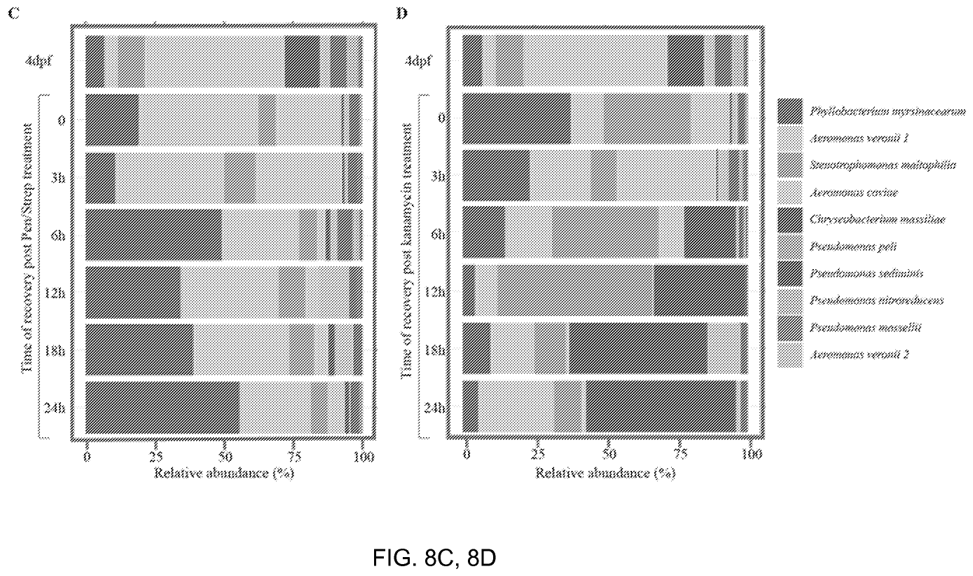

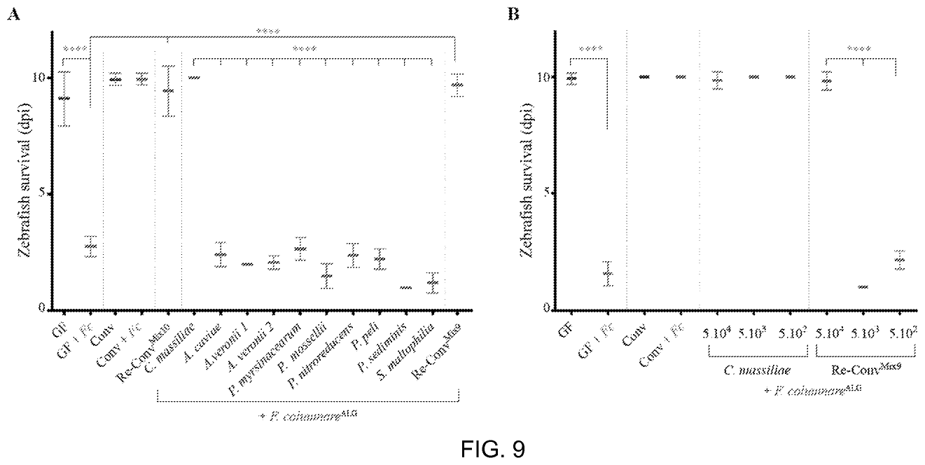

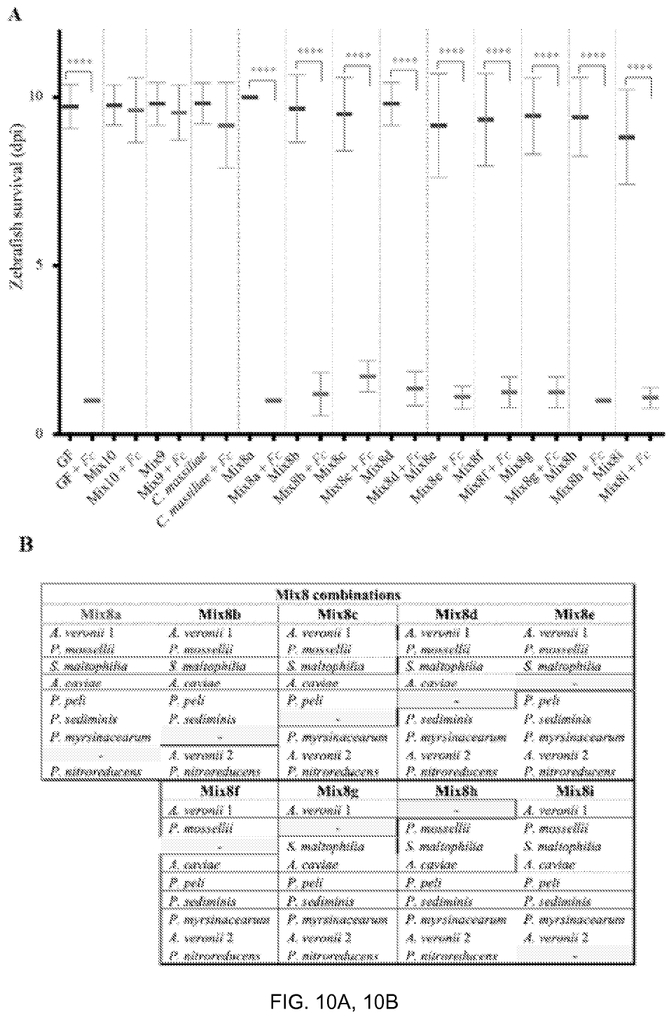

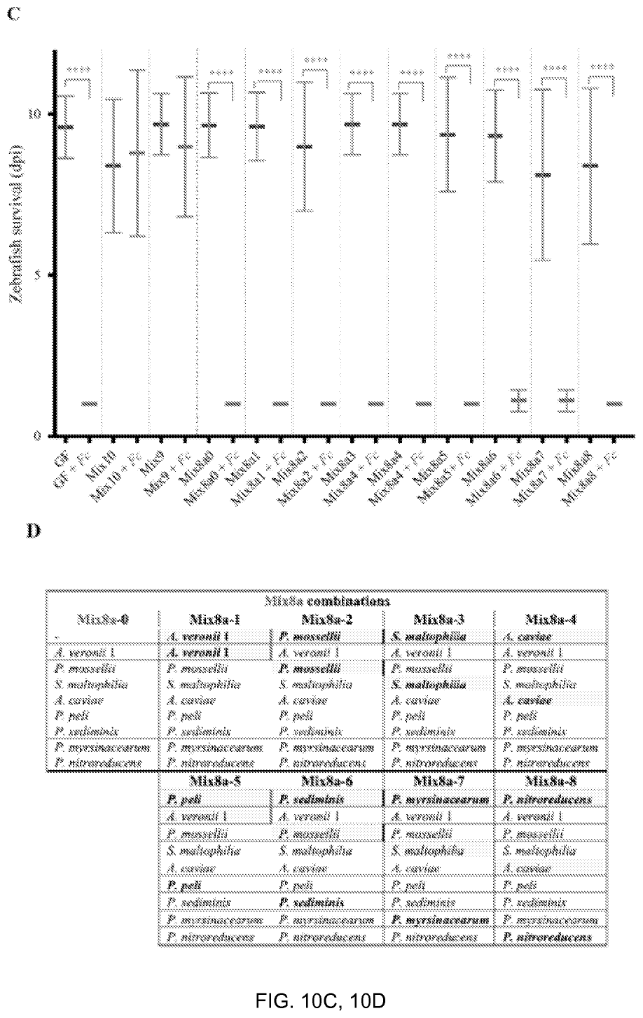

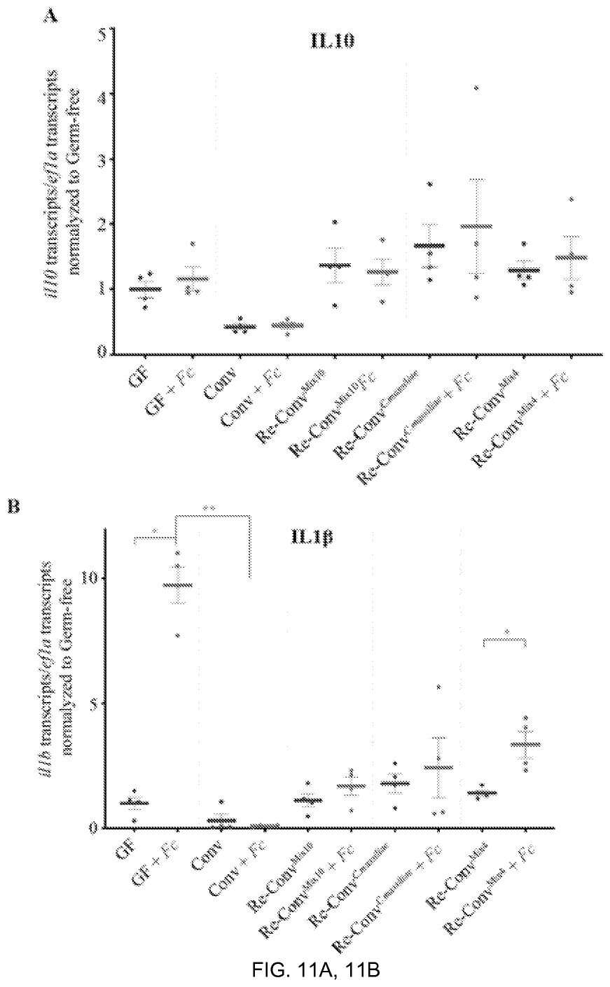

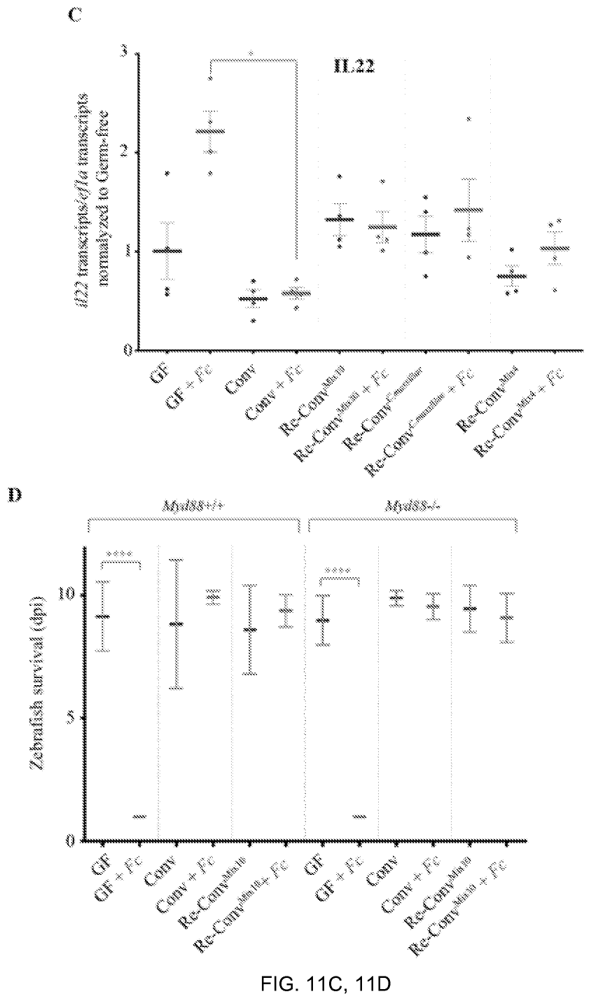

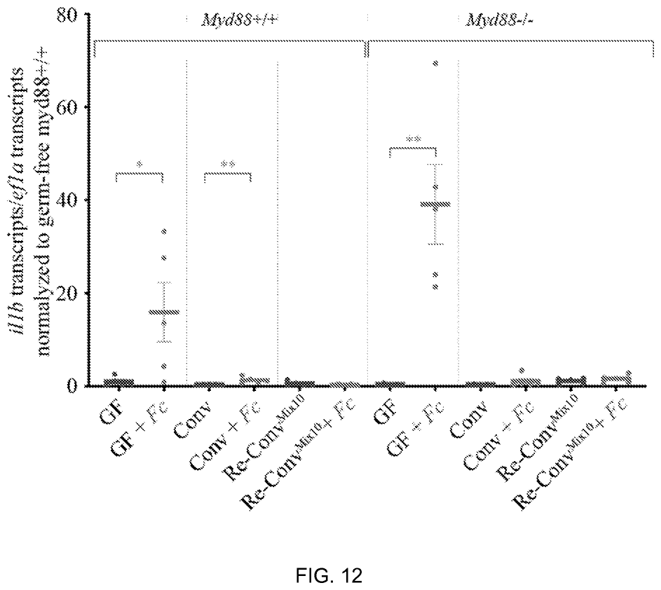

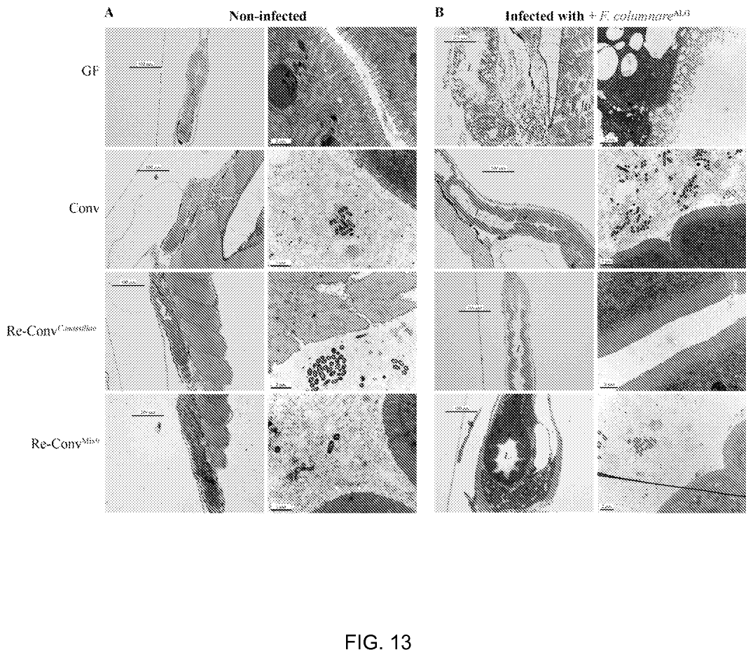

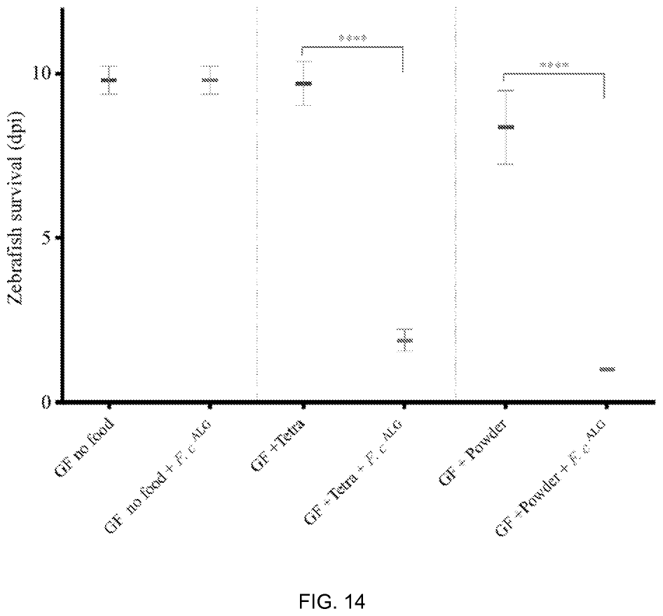

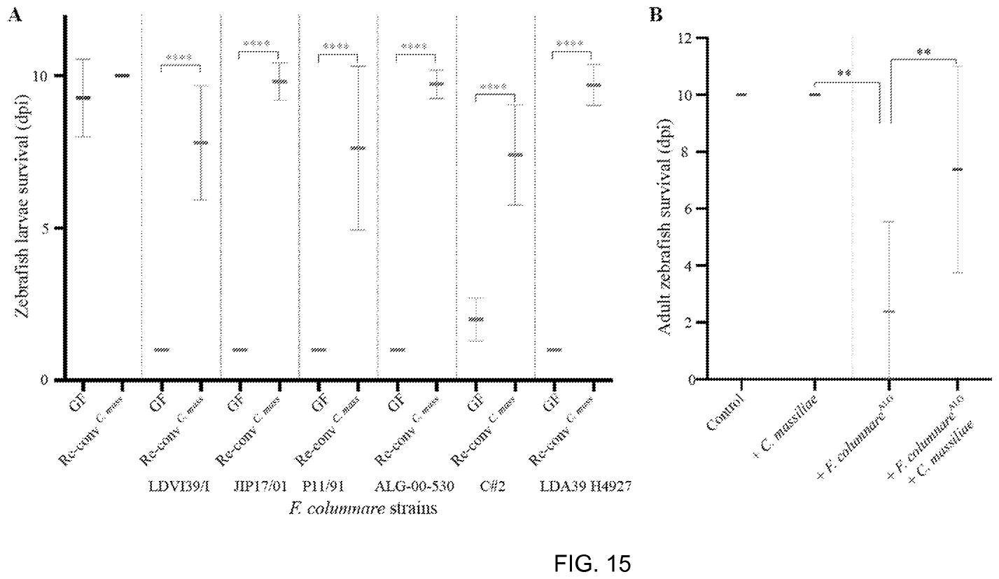

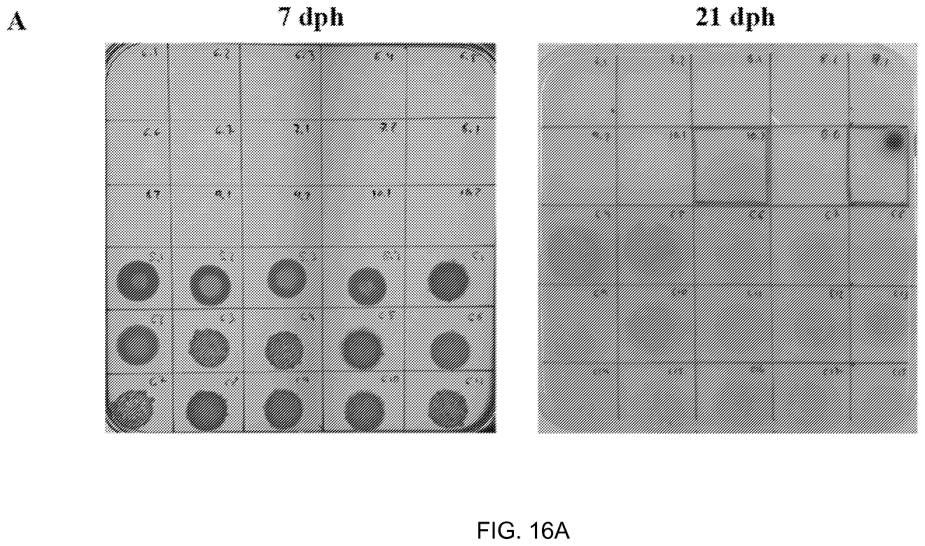

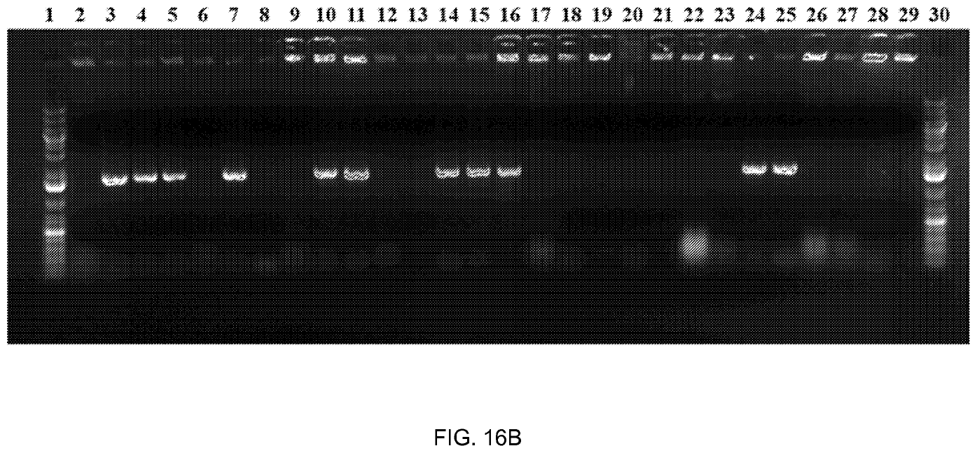

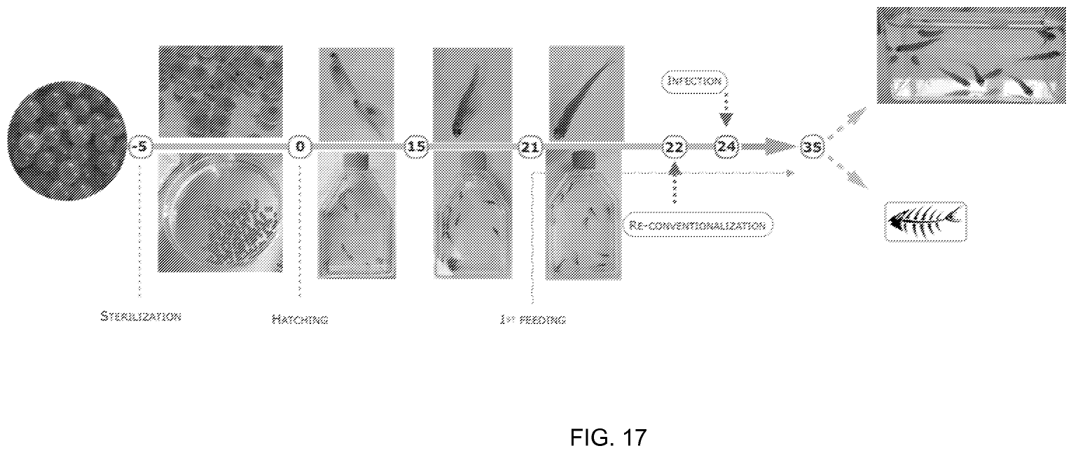

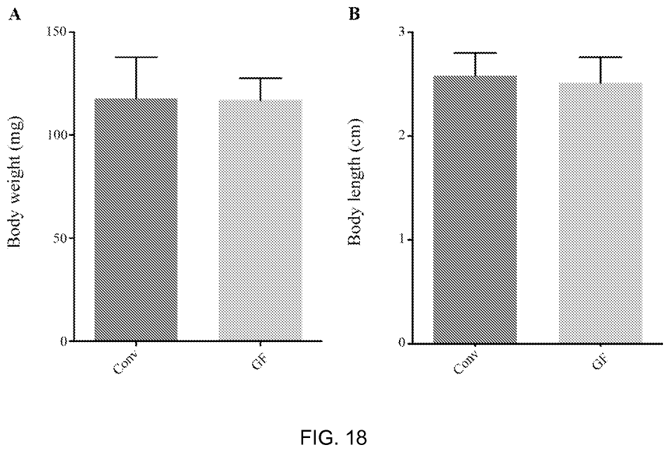

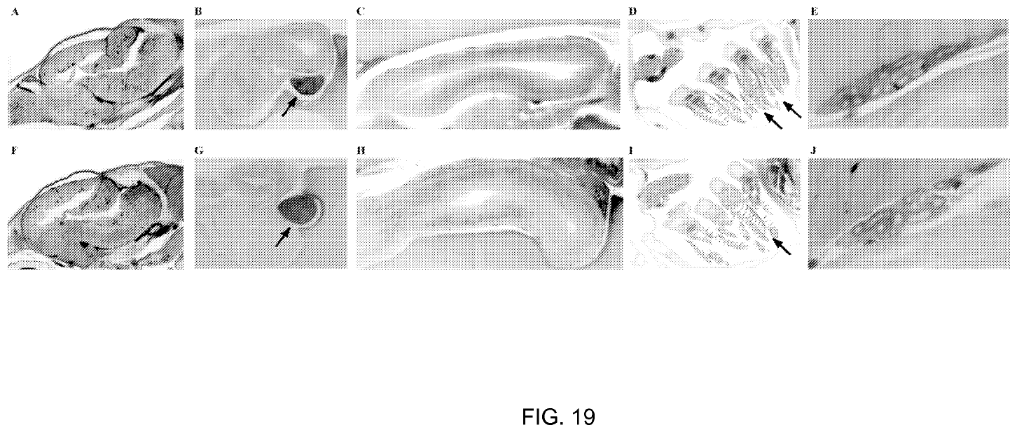

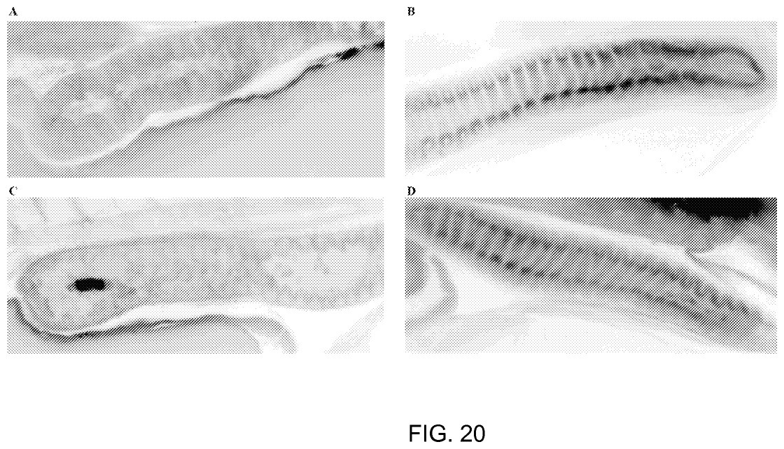

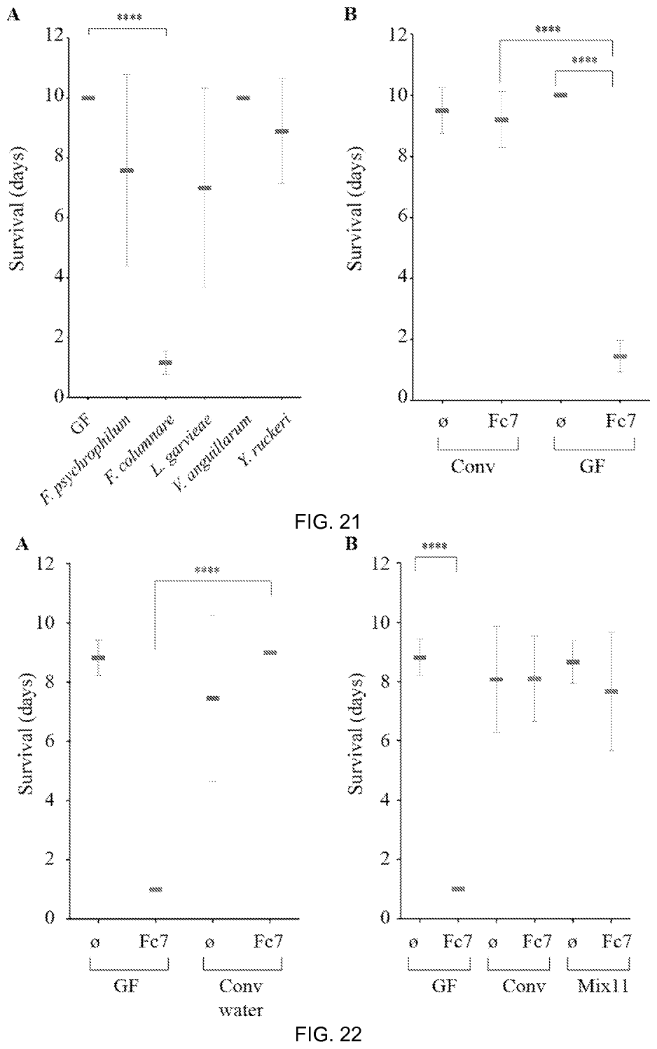

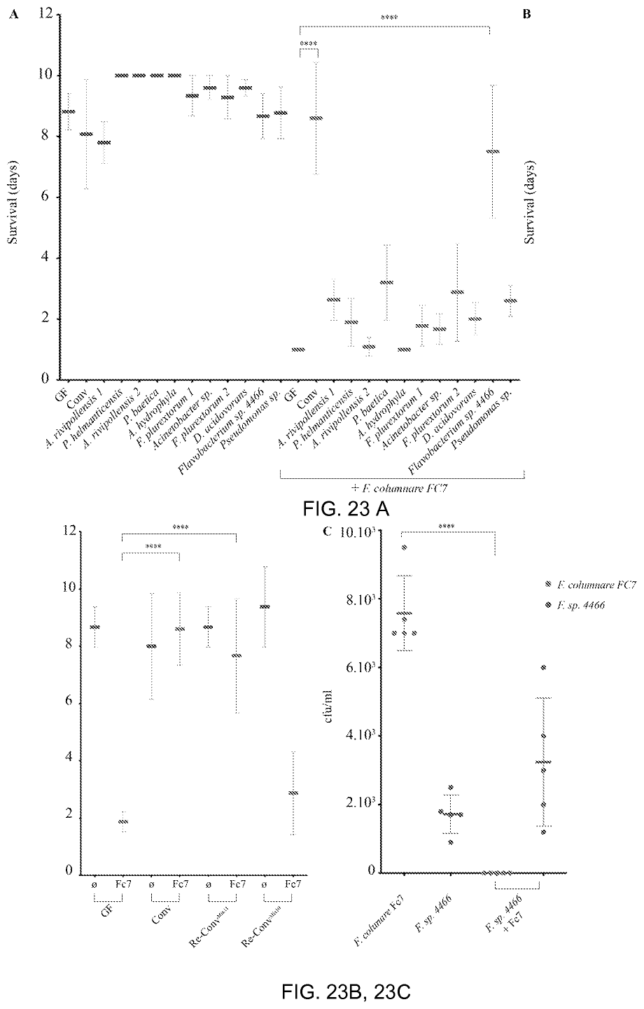

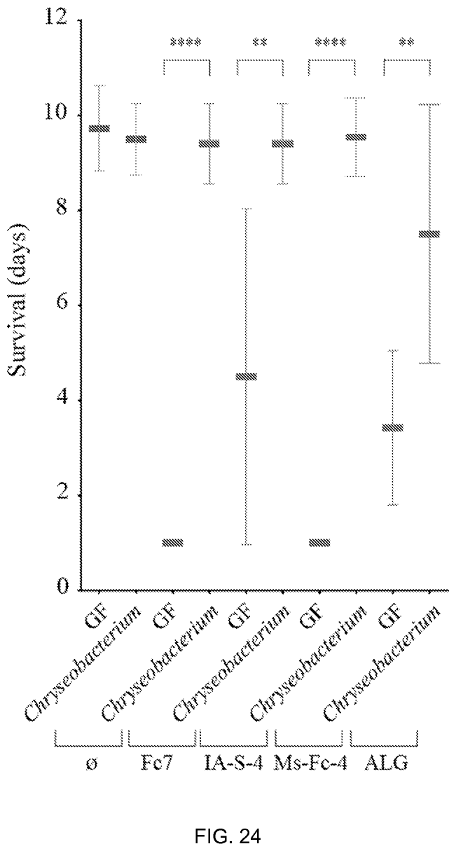

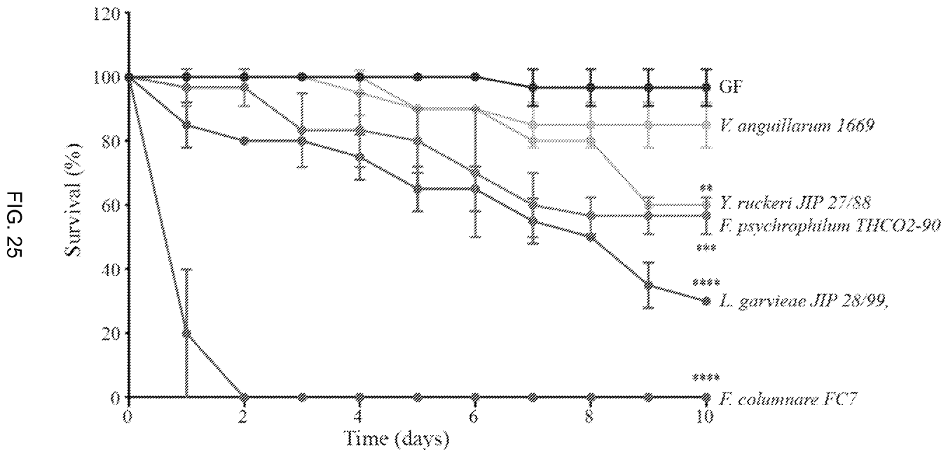

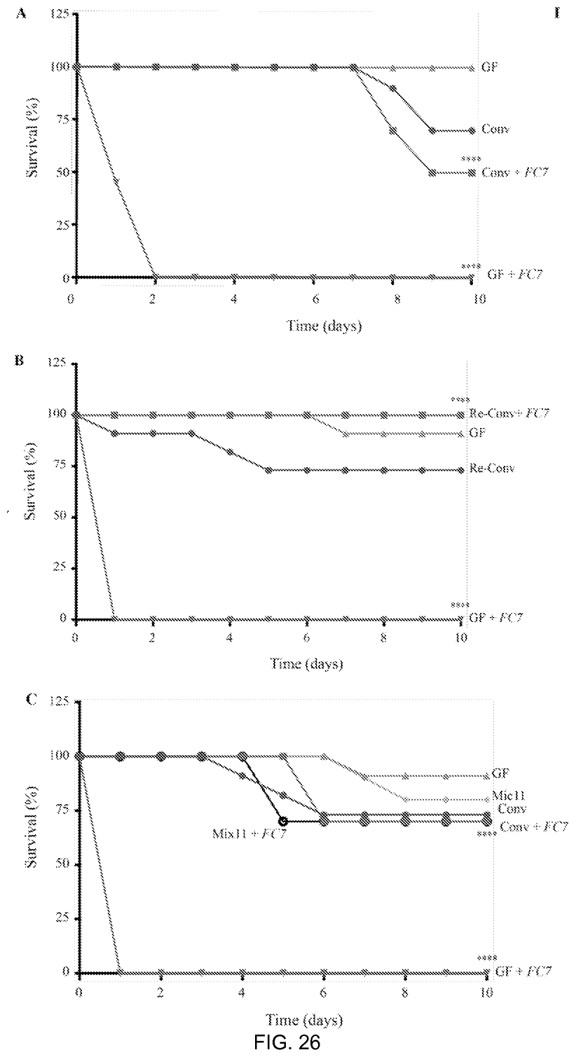

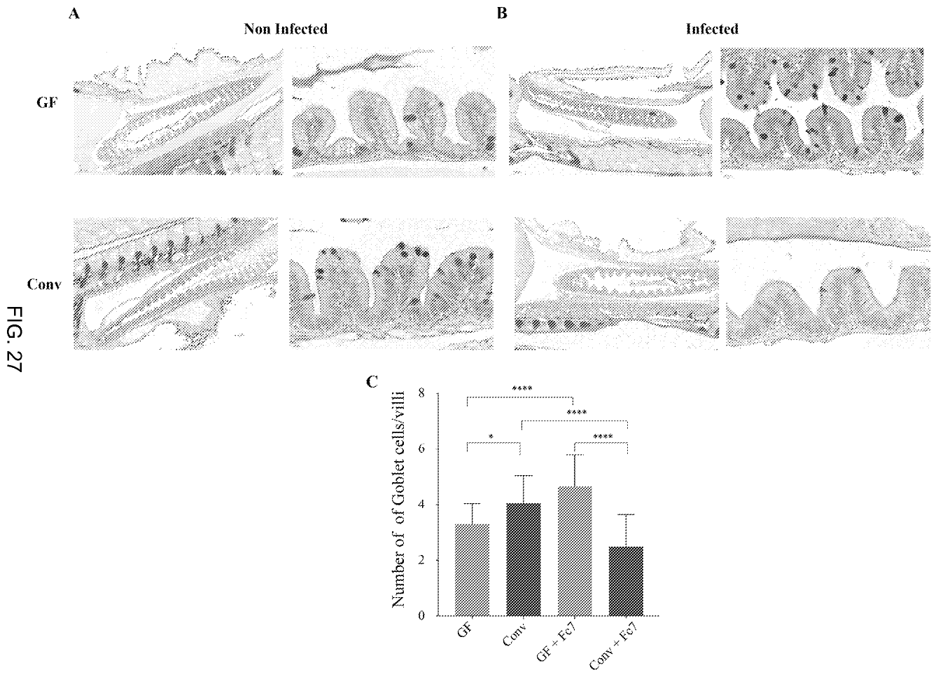

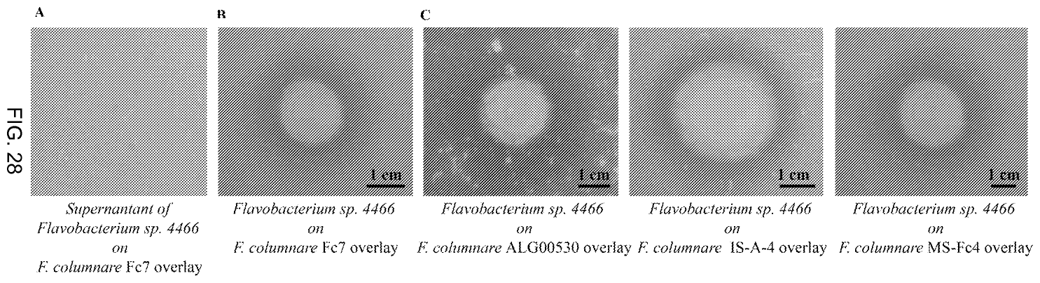

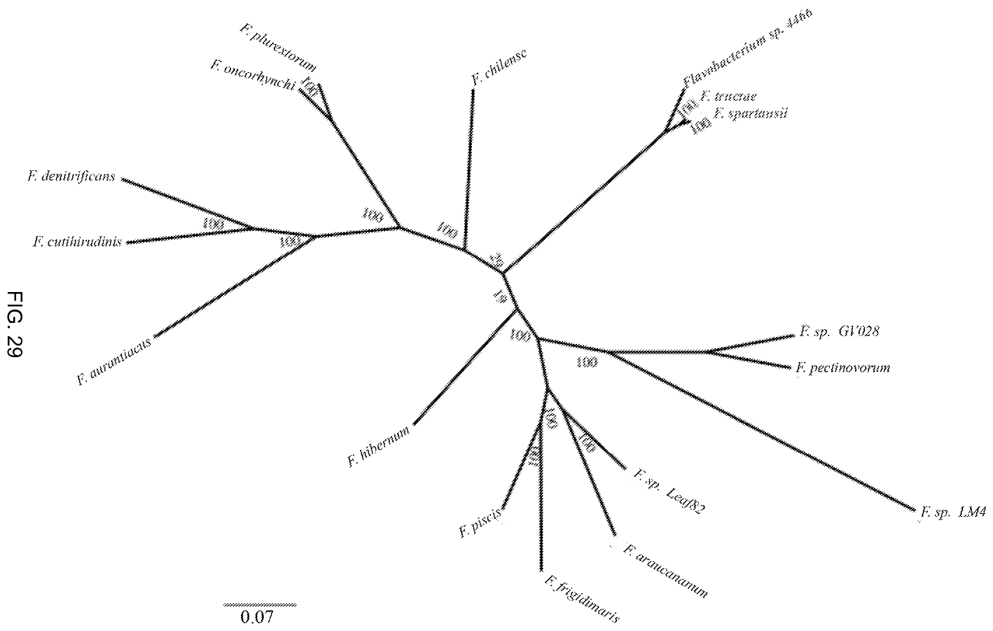

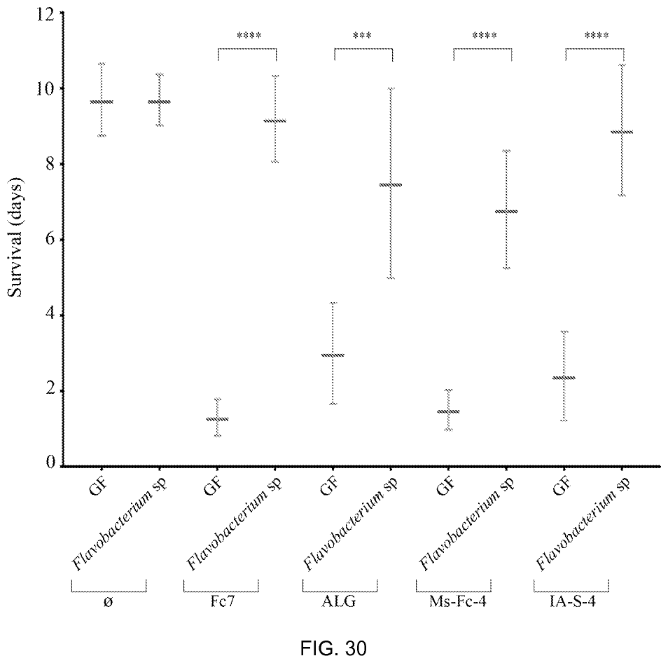

To investigate microbiota-based resistance to infection in zebrafish, we compared the sensitivity of germ-free (GF) and conventional (Conv) zebrafish larvae to F. columnare, an important fish pathogen previously shown to infect and kill adult zebrafish [33, 34]. We used bath immersion to expose GF and Conv zebrafish larvae at 6 days post fertilization (dpf), to a collection of 28 F. columnare strains, belonging to four different genomovars for 3 h at 5.10⁵ colony forming unit (cfu)/mL. Daily monitoring showed that 16 out of 28 F. columnare strains killed GF larvae in less than 48 h (FIG. 2A), whereas Conv larvae survived exposure to all tested virulent F. columnare strains (FIG. 2B). Exposure to the highly virulent strain ALG-00-530 (hereafter F. columnare^{ALG}) also showed that GF mortality was rapid (1 day) and dose-dependent and that Conv zebrafish survived all but the highest dose (10⁷ cfu/mL) (FIG. 1). Similar survival of infected Conv larvae was obtained with zebrafish AB strain eggs obtained from 4 different zebrafish facilities (FIG. 3), suggesting that conventional zebrafish microbiota could provide protection against F. columnare infection.

Ten Culturable Bacterial Strains are Sufficient to Protect Against F. columnare Infection

In our rearing conditions, the conventional larval microbiota is acquired after hatching from microorganisms present on the egg chorion and in fish facility water. To test the hypothesis that microorganisms associated with conventional eggs provided protection against F. columnare^{ALG}, we exposed sterilized eggs to either fish facility tank water or to mashed non-sterilized conventional eggs at 0 or 4 dpf (before or after hatching, respectively). In both cases, these re-conventionalized (re-Conv) zebrafish survived F. columnare^{ALG} infection as well as Conv zebrafish (FIG. 2). To determine the composition of conventional zebrafish microbiota, we generated a clone library and sequenced the 16S rDNA gene content of homogenate pools of 10 Conv zebrafish larvae aged 4, 6 and 11 dpf, exposed or not to F. columnare^{ALG}, sampled over 3 months from different batches of larvae. We identified 13 predominant OTUs, 10 of which were identified in all experiments (Table 1). Moreover, deep-sequencing of the 16S rDNA V3-V4 region of gDNA retrieved from larvae originating from the other four zebrafish facilities described above revealed that most of these 10 OTUs were also detected in Conv larvae (Table 2).

TABLE 2
Percent abundance of OTUs detected by Illumina 16S rDNA sequencing for C. massiliae, F. columnare
and other strains isolated at Institut Pasteur facility as “core” microbiota in facilities F1-F4.

| | F1 | | | | F2 | | | | F3 | | | | F4 | | | |
|---|---|---|---|---|---|---|---|---|---|---|---|---|---|---|---|---|
| | | | +F. | | | | +F. | | | | +F. | | | | + F. | |
| | uninfected | | columnare | | uninfected | | columnare | | uninfected | | columnare | | uninfected | | columnare | |
| | 6 | 11 | 6 | 11 | 6 | 11 | 6 | 11 | 6 | 11 | 6 | 11 | 6 | 11 | 6 | 11 |
| | dpf | dpf | dpf | dpf | dpf | dpf | dpf | dpf | dpf | dpf | dpf | dpf | dpf | dpf | dpf | dpf |
| Aeromonas | | | | | | | | | 0.01 | | 0.01 | | | | | |
| hydrophila | | | | | | | | | | | | | | | | |
| Aeromonas spp. | | | | | 0.04 | 0.09 | 15.32 | 8.93 | | | 0.01 | | 0.70 | | | |
| Chryseobacterium | 0.64 | 0.03 | 1.07 | | 43.13 | 13.45 | 9.03 | 11.52 | | | | | 36.08 | 1.71 | 53.06 | 1.68 |
| massiliae | | | | | | | | | | | | | | | | |
| Chryseobacterium | | | | | 0.02 | | | | | | | | <0.01 | | <0.01 | |
| spp. | | | | | | | | | | | | | | | | |
| Flavobacterium | | | 0.66 | | | | 0.08 | | 0.01 | | 0.85 | | | | 3.97 | |
| columnare | | | | | | | | | | | | | | | | |
| Flavobacterium | 0.21 | 8.42 | 0.25 | 8.75 | | 0.10 | | | 66.61 | 31.36 | 60.68 | 21.55 | | | | |
| spp. | | | | | | | | | | | | | | | | |
| Phyllobacterium | | | | | | | | | | | | | | | | |
| sp. | | | | | | | | | | | | | | | | |
| Pseudomonas | | | | | 0.01 | 0.05 | | 0.01 | 1.16 | 0.03 | 2.25 | 19.49 | | | <0.01 | |
| (pseudo)alcaligenes | | | | | | | | | | | | | | | | |
| Pseudomonas | | 5.68 | | 0.10 | | | | | | <0.01 | | | 4.03 | 6.72 | 15.06 | 28.75 |
| mossellii | | | | | | | | | | | | | | | | |
| Pseudomonas peli | | | | | 6.98 | 0.10 | 4.24 | 0.07 | 6.69 | 1.27 | 18.39 | 3.17 | | | | |
| Pseudomonas spp. | 15.64 | 1.67 | 14.77 | 14.70 | 0.08 | 0.12 | 0.14 | 0.03 | 0.31 | | 0.90 | 5.67 | 0.29 | 0.60 | 0.65 | 1.28 |
| Stenotrophomonas | 0.21 | | | | | | | | | | | | | | | |
| maltophilia | | | | | | | | | | | | | | | | |
| Stenotrophomonas | 0.32 | 0.06 | 0.25 | | | 0.02 | 0.01 | | | | | 0.05 | 0.01 | | | |
| sp. | | | | | | | | | | | | | | | | |

| | F1 | F1 | F2 | F2 | F3 | F3 | F4 | F4 | our facilty | our facilty |
|---|---|---|---|---|---|---|---|---|---|---|
| | conv | conv | conv | conv | conv | conv | conv | conv | conv | conv |
| | 0 dpi | 5 dpi | 0 dpi | 5 dpi | 0 dpi | 5 dpi | 0 dpi | 5 dpi | 0 dpi | 5 dpi |
| Aeromonas aquatica | 0.56 | 0.33 | 0.98 | 13.79 | 1.97 | 0.12 | 36.18 | 0.79 | 3.46 | 14.53 |
| Aeromonas veronii 1 | 9.30 | 3.15 | 0.62 | 1.38 | 0.50 | 1.26 | 1.17 | 45.27 | 73.18 | 4.17 |
| Aeromonas veronii 2 | 0.07 | 0.04 | 0.00 | 0.01 | 0.00 | 0.00 | 0.00 | 0.02 | 0.00 | 0.00 |
| Chryseobacterium massilliae | 0.73 | 0.11 | 49.13 | 17.15 | 0.01 | 0.00 | 30.62 | 2.72 | 1.47 | 0.89 |
| Phyllobacterium myrsinacearum | 5.00 | 5.16 | 1.70 | 11.05 | 13.78 | 12.56 | 3.10 | 1.39 | 5.63 | 22.55 |
| Pseudomonas alcaliphila | 0.17 | 0.56 | 2.31 | 2.67 | 1.06 | 0.05 | 0.39 | 0.92 | 3.77 | 2.51 |
| Pseudomonas mossellii | 21.83 | 9.26 | 0.02 | 0.18 | 1.70 | 1.13 | 8.67 | 13.38 | 2.14 | 36.58 |
| Pseudomonas nitrireducens | 0.24 | 1.35 | 0.56 | 29.27 | 7.12 | 15.26 | 2.83 | 1.92 | 0.00 | 4.66 |
| Pseudomonas peli | 57.01 | 76.51 | 41.82 | 21.72 | 68.02 | 66.07 | 2.99 | 2.43 | 1.99 | 2.83 |
| Stenotrophomonas mallophila | 4.20 | 3.37 | 2.65 | 2.67 | 5.73 | 3.52 | 14.00 | 31.02 | 8.11 | 11.30 |

To isolate culturable zebrafish microbiota bacteria, we plated dilutions of homogenized 6 dpf and 11 dpf larvae pools on various growth media and we identified 10 different bacterial morphotypes. Use of 16S-based analysis followed by full genome sequencing identified 10 bacteria corresponding to 10 strains of 9 different species that were also consistently detected by culture-free approaches (Table 1). We then re-conventionalized GF zebrafish at 4 dpf with a mix of all 10 identified culturable bacterial species (hereafter called Mix10), each at a concentration of 5.10$ cfu/mL and we monitored zebrafish survival after exposure to F. columnare^{ALG} at 6 dpf. We showed that zebrafish reconventionalized with the Mix10 (Re-Conv^{Mix10}) displayed strong level of protection against all identified highly virulent F. columnare strains (FIG. 5). These results demonstrated that the Mix10 constitute a core protective bacterial community providing full protection of zebrafish larvae against F. columnare infection.

TABLE 1
The 10 strains composing the core of zebrafish larvae microbiota
Bacterial strains consistently detected at all time points
(4, 6 and 11 dpf) in all experiment runs and constituting the core of
conventional zebrafish larval microbiota and their taxonomic affiliation.

| Bacterial species of the core | | | | |
|---|---|---|---|---|
| zebrafish microbiota | ANI %^{a} | 16S rRNA (%)^{b} | recA (%)^{c} | rpIC (%)^{d} |
| Aeromonas veronii 1 | 96.52 | 98.27 | 97.00 | 99.84 |
| Aeromonas veronii 2 | 96.58 | 99.53 | 98.31 | 99.68 |
| Aeromonas caviae | 97.97 | 99.94 | 98.78 | 99.84 |
| Chryseobacterium massiliae | 95.85 | 99.86 | 96.61 | 99.84 |
| Phyllobacterium myrsinacearum | 98.58 | 99.86 | 99.72 | 100 |
| Pseudomonas sediminis | 96.12 | 99.73 | 97.70 | 99.84 |
| Pseudomonas mossellii | 99.39 | 98.27 | 100 | 99.84 |
| Pseudomonas nitroreducens* | 92.14 | 99.80 | 94.95 | 99.06 |
| Pseudomonas peli* | 88.84 | 99.20 | 91.51 | 95.44 |
| Stenotrophomas maltophilia* | 90.94 | 97.85 | 95.38 | 99.08 |

^{a}Average Nucleotide Identity value
^{b}16S rRNA gene sequence similarity
^{c}recA gene sequence similarity
^{d}rpIC gene sequence similarity
*Species ambiguously identified

Community Dynamics Under Antibiotic Dysbiosis Reveal a Key Contributor to Resistance to F. columnare Infection

To further analyze the determinants of Mix10 protection against F. columnare^{ALG} infection, we inoculated 4 dpf larvae with an equal-ratio mix of the 10 bacteria (at 5.10⁵ cfu/mL each) and monitored their establishment over 8 hours. We first verified that whole larvae bacterial content was not significantly different from content of dissected intestinal tubes (p=0.99). We then collected pools of 10 larvae immediately after reconventionalization (t₀), 20 min, 2 hours, 4 hours and 8 hours and we used 16S rDNA sequencing to follow bacterial relative abundance. At t₀, all species were present at >4% in the zebrafish, apart from A. veronii strains 1 (0.2%) and 2 (not detected) (FIG. 6). Aeromonas caviae was detected as the most abundant species, followed by Stenotrophomonas maltophilia and Chryseobacterium massiliae, altogether composing 67.5% of the community (FIG. 6). The relative species abundance, possibly reflecting initial colonization ability, was relatively stable for most species during community establishment, with similar species evenness at t₀ (E=0.84) and t₈h (E=0.85). However, whereas both Conv and Re-Conv^{Mix10} larvae were protected against F. columnare^{ALG} infection, the global structure of the reconstituted Mix10 population was different from the conventional one at 4 dpf (FIG. 6). To test the sensitivity to disturbance and the resilience of the protection provided by Mix10 bacterial community, we subjected Re-Conv^{Mix10} zebrafish to non-toxic antibiotic treatment at 4dpf using either 250 μg/mL penicillin/streptomycin combination (all members of the Mix10 bacteria are sensitive to penicillin/streptomycin) or 50 μg/mL kanamycin (affecting all members of the Mix10 bacteria except C. massiliae, P. myrsinacearum and S. maltophilia) (FIG. 7). At 5 dpf, after 16 hours of exposure, antibiotics were washed off and zebrafish were immediately exposed to F. columnare^{ALG}. Both antibiotic treatments resulted in complete loss of the protection against F. columnare^{ALG} infection observed in Re-Conv^{Mix10} (FIG. 8A). We then used the same antibiotic treatments but followed by a 24 h recovery period after washing off the antibiotics at 5 dpf, therefore only performing the infection at 6 dpf (FIG. 8B). Whilst Re-Conv^{Mix10} larvae treated with penicillin/streptomycin still showed similar protection to infected GF larvae, kanamycin-treated Re-Conv^{Mix10} zebrafish showed restored protection after 24h recovery and survived similarly to untreated conventionalized fish (FIG. 8B). Sampling and 16S analysis during recovery experiments at different time points showed that bacterial community evenness decreased similarly after antibiotic administration for both treatments (E=0.85 for 4 dpf control, E=0.72 for t₀ kanamycin and E=0.7 for t₀ penicillin/streptomycin), and continued to decrease during recovery (E=0.6 and 0.64 for kanamycin and penicillin/streptomycin treatment after 24h recovery, respectively). Remarkably, although C. massiliae remained similarly detectable immediately after both antibiotic treatments, penicillin/streptomycin treatment led a significant reduction in its relative abundance (FIG. 8C). By contrast, C. massiliae relative abundance rebounded 6h after cessation of kanamycin treatment and was the dominant member of the reconstituted microbiota after 24h recovery period (FIG. 8D), suggesting that the protective effect observed in the kanamycin-treated larvae might be due to the recovery of C. massiliae.

Resistance to F. columnare Infection is Provided by Both Individual- and Community-Level Protection

To test the potential key role played by C. massiliae in protection against F. columnare^{ALG} infection, we exposed 4 dpf GF zebrafish to C. massiliae only and showed that it conferred individual protection at doses as low as 5.10² cfu/mL (FIG. 9). Interestingly, although none of the 9 other species composing the Mix10 were protective individually (FIG. 9A), their equiratio combination (designated as Mix9) conferred protection to zebrafish, although not at doses lower than 5.10⁴ cfu/mL and not as reproducibly as with C. massiliae (FIG. 9B). To identify which association of species or species combination protected Re-Conv^{Mix9} zebrafish against F. columnare infection, we tested all 9 combinations of 8 species (Mix8), as well as several combinations of 7.6.4 or 3 species and showed no protection (FIG. 10 and Table 3). We then tested whether lack of protection of Mix8 compared to Mix9 could rely on a density effect by doubling the concentration of any of the species within the non-protective Mix8a (FIG. 10B) and showed no protection. These results therefore indicated that microbiota-based protection against F. columnare infection relied on either C. massiliae-dependent membership effect or on a community-dependent effect mediated by the Mix9 consortium.

TABLE 3
Combinations of 3, 4, 6 and 7 of the core zebrafish microbiota species tested for
their ability to protect against infection by F. columnare^{ALG}.
These tested combinations did not include C. massiliae.
None of these tested combinations showed significant protection activity against F. columnare^{ALG}.

Tested 3 species combinations
A. caviae + A. veronii2 + A. veronii1
P. myrsinacearum + P. mosselli + P. nitroreducens
P. peli + P. sediminis + S. maltophilia
A. caviae + A. veronii1 + P. myrsinacearum
A. caviae + A. veronii1 + P. nitroreducens
A. caviae + P. myrsinacearum + P. nitroreducens
A. caviae + P. nitroreducens + P. sediminis
A. caviae + P. mosselli + P. peli
A. caviae + P. peli + S. maltophilia
A. caviae + P. mosselli + P. nitroreducens
A. caviae + A. veronii2 + P. myrsinacearum
A. caviae + P. myrsinacearum + S. maltophilia
A. veronii2 + P. mosselli + P. peli
A. veronii2 + P. peli + S. maltophilia
A. veronii2 + P. myrsinacearum + P. nitroreducens
A. veronii2 + P. peli +S. maltophilia
A. veronii2 + P. nitroreducens + P. sediminis
A. veronii1 + P. myrsinacearum + P. mosselli
A. veronii1 + P. mosselli + P. peli
A. veronii1 + P. nitroreducens + S. maltophilia
A. veronii1 + P. myrsinacearum + P. peli
A. veronii1 + P. myrsmacearum + S. maltophilia
P. myrsinacearum + P. nitroreducens + P. sediminis
P. myrsinacearum + P. peli + S. maltophilia
P. mosselli + P. peli + S. maltophilia
A. caviae + P. nitroreducens + S. maltophilia
A. veronii2 + A. veronii1 + P. sediminis
A. veronii2 + P. mosselli + P. peli
A. caviae + P. peli + P. nitroreducens
Tested 4 species combinations
A. caviae + A. veronii2 + A. veronii1 + C. massiliae
A. caviae + A. veronii2 + A. veronii1 + P. mosselli
C. massiliae + P. myrsinacearum + P. mosselli + P. nitroreducens
C. massiliae + P. peli + P. sediminis + S. maltophilia
A. caviae + A. veronii2 + P. myrsinacearum + P. mosselli
A. caviae + A. veronii2 + P. nitroreducens + P. peli
A. caviae + A. veronii2 + P. sediminis + S. maltophilia
P. myrsinacearum + P. mosselli + P. nitroreducens + P. peli
A. caviae + P. mosselli + P. nitroreducens + P. peli
A. veronii2 + P. mosselli + P. nitroreducens + P. peli
A. veronii2 + P. peli + P. sediminis + S. maltophilia
P. myrsinacearum + P. mosselli + P. sediminis + S. maltophilia
A. caviae + A. veronii2 + P. mosselli + P. sediminis
A. caviae + A. veronii2 + P. nitroreducens + S. maltophilia
A. caviae + A. veronii2 + P. myrsinacearum + P. peli
A. caviae + P. myrsinacearum+ P. nitroreducens + P. sediminis
A. veronii2 + A. veronii1 + P. myrsinacearum + P. mosselli
A. veronii2 + P. myrsinacearum + P. nitroreducens + P. peli
A. veronii2 + P. myrsmacearum + P. sediminis + S. maltophilia
A. veronii2 + P. myrsinacearum + P. peli + S. maltophilia
A. veronii2 + A. veronii1 + P. mosselli + P. nitroreducens
A. veronii2 + A. veronii1 + P. peli + P. sediminis
A. veronii2 + A. veronii1 + P. nitroreducens + S. maltophilia
A. veronii2 + A. veronii1 + P. myrsinacearum + P. sediminis
A. veronii2 + A. veronii1 + P. myrsinacearum + S. maltophilia
A. veronii2 + A. veronii1 + P. mosselli + P. sediminis
A. veronii2 + A. veronii1 + P. nitroreducens + P. peli
A. veronii2 + A. veronii1 + P. mosselli + S. maltophilia
A. veronii1 + P. myrsinacearum + P. mosselli + P. nitroreducens
A. veronii1 + P. myrsinacearum + P. mosselli + P. peli
A. veronii1 + P. myrsinacearum + P. mosselli + P. sediminis
A. veronii1 + P. myrsinacearum + P. mosselli + S. maltophilia
A. veronii1 + P. nitroreducens + P. peli + S. maltophilia
A. veronii1 + P. peli + P. sediminis + S. maltophilia
A. veronii1 + P. mosselli + P. peli + S. maltophilia
A. veronii1 + P. nitroreducens + P. peli + S. maltophilia
P. mosselli + A. caviae + P. peli + P. nitroreducens
A. caviae + P. peli + A. veronii2 + P. nitroreducens
Tested 6 species combinations
A. caviae + P. myrsinacearum + P. nitroreducens + P. peli + P. sediminis + S. maltophilia
A. caviae + A. veronii1 + P. mosselli + P. nitroreducens + P. peli + S. maltophilia
A. veronii2 + A. veronii1 + P. mosselli + P. peli + P. sediminis + S. maltophilia
A. veronii2 + A. veronii1 + P. myrsinacearum + P. nitroreducens + P. sediminis + S. maltophilia
A. veronii2 + P. myrsmacearum + P. mosselli + P. nitroreducens + P. peli + S. maltophilia
A. veronii1 + P. myrsinacearum + P. mosselli + P. nitroreducens + P. peli + P. sediminis
A. veronii1 + P. myrsinacearum + P. mosselli + P. nitroreducens + P. peli + S. maltophilia
A. veronii1 + P. mosselli + P. nitroreducens + P. peli + P. sediminis + S. maltophilia
P. myrsinacearum + P. mosselli + P. nitroreducens + P. peli + P. sediminis + S. maltophilia
S. maltophilia + A. caviae + P. peli + P. sediminis + P. myrsinacearum + P. nitroreducens
Tested 7 species combinations
A. caviae + A. veronii2 + A. veronii1 + P. myrsinacearum + P. mosselli + P. nitroreducens + P. peli
A. caviae + A. veroni2 + A. veronii1 + P. myrsinacearum + P. mosselli + P. nitroreducens + P. sediminis
A. caviae + A. veronii2 + A. veronii1 + P. myrsinacearum + P. mosselli + P. nitroreducens + S. maltophilia
A. caviae + A. veronii2 + A. veronii1 + P. myrsinacearum + P. mosselli + P. peli + P. sediminisa
A. caviae + A. veronii2 + A. veronii1 + P. myrsinacearum + P. mosselli + P. sediminis + S. maltophilia
A. veronii2 + A. veronii1 + P. myrsinacearum + P. mosselli + P. nitroreducens + P. peli + P. sediminis

Pro- and Anti-Inflammatory Cytokine Production do not Contribute to Microbiota-Mediated Protection Against F. columnare^{ALG} Infection.

To test the contribution of larval zebrafish's innate immune response to resistance to F. columnare infection, we used qRT-PCR to measure cytokine mRNA expression in GF and Conv zebrafish, as well as in larvae reconventionalized with C. massiliae (re-Conv^{Cm}), Mix10 (re-Conv^{Mix10}) or with Mix4 (A. caviae, both A. veronii spp., P. mossellii) as a non-protective control (Table 3), exposed or not to F. columnare^{ALG}. We tested genes encoding IL1β (pro-inflammatory), IL22 (promoting gut repair), and IL10 (anti-inflammatory). While we observed some variation in il10 expression among non-infected reconventionalized larvae, this did not correlate with protection. Furthermore, il10 expression was not modulated by infection in any of the tested conditions (FIG. 11A). By contrast, we observed a strong induction of il1b and il22 in GF zebrafish exposed to F. columnare^{ALG} (FIG. 11BC). This induction, however, was not seen in protected Conv, Re-Conv^{Cm}, Re-Conv^{Mix10} or non-protective Re-Conv^{Mix4} larvae. These observations indicate that the presence of a gut microbiota significantly (down-)modulates the inflammatory response induced by F. columnare infection. However, this effect does not correlate with protection, suggesting that inflammation modulation is not the main mechanism of the microbiota-induced resistance. Considering that Myd88 is a key adaptor downstream of IL-1 and toll-like receptor signaling, we also generated GF and Conv larvae from myd88 null zebrafish mutants and exposed them to F. columnare^{ALG} infection. Remarkably, despite their deficiency in innate immunity, Conv or Re-Conv^{Mix10}, but not GF myd88 mutants, survived F. columnare as well as wild-type zebrafish (FIG. 11D). Moreover, il1b induction by F. columnare infection was observed only in GF larvae and was myd88-independent (FIG. 12). These results are therefore inconsistent with a significant role of cytokine contribution to microbiota-mediated protection against F. columnare infection by modulation of the pathogen-induced inflammatory response.

C. massiliae and Mix9 Protect Zebrafish from Intestinal Damage Upon F. columnare Infection

Histological analysis of GF larvae fixed 24 h after exposure to F. columnare^{ALG} revealed extensive intestinal damage (FIG. 13A) prior to noticeable signs in other potential target organs such as gills or skin. To test the requirement for gut access in F. columnare^{ALG} infection process, we modified our standard rearing protocol of GF fish, which involves feeding with live germ-free T, thermophila. We found that, if left unfed, GF zebrafish did not die after F. columnare^{ALG} exposure, while feeding with either T. thermophila or another food source such as sterile fish food powder, restored sensitivity to F. columnare^{ALG} infection (FIG. 14), suggesting that successful infection requires feeding and ingestion.

Histological sections consistently showed severe disorganization of the intestine with blebbing in the microvilli and vacuole formation in F. columnare-infected GF larvae (FIG. 13). In contrast, zebrafish pre-incubated with either C. massiliae or Mix9 consortium at 4 dpf, and then exposed to F. columnare at 6 dpf showed no difference compared to non-infected larvae or conventional infected larvae (FIG. 13), confirming full protection against F. columnare at the intestinal level.

C. massiliae Protects Larvae and Adult Zebrafish Against Virulent F. columnare Strains

The clear protection provided by C. massiliae against F. columnare infection prompted us to test whether exogenous addition of this bacterium could improve microbiota-based protection towards this widespread fish pathogen. We first showed that zebrafish larvae colonized with C. massiliae were fully protected against all virulent F. columnare strains identified in this study (FIG. 15A). To test whether C. massiliae could also protect adult zebrafish from F. columnare infection, we pre-treated conventional 3-4 month old Conv adult zebrafish with C. massiliae for 48 h before challenging them with a high dose (5×10⁶ cfu/mL) F. columnare^{ALG}. Monitoring of mortality rate showed that pre-treatment with C. massiliae significantly increased (p=0.0084)) the survival rate of adult zebrafish upon F. columnare^{ALG} infection compared to non-treated conventional fish (FIG. 15B). Taken together, these results show that C. massiliae is a putative broad-spectrum probiotic protecting zebrafish against columnaris disease caused by F. columnare.

### Generation of Germ-Free Rainbow Trout Larvae.

To investigate the potential protection conferred by endogenous or exogenous bacteria against incoming pathogens in microbiologically controlled rainbow trout host (Oncorhynchus mykiss), we first aimed to produce germ-free (GF) trout larvae. For this, we exposed freshly fertilized eggs for 5 h to a previously described cocktail of antibiotics and antifungal [136], and then to 0.005% bleach for 15 min, followed by a 10 min treatment with Romeiod, a iodophore disinfection solution. Germ-free eggs were then kept under sterile conditions in class II hood in a 16° ° C. aqueous solution supplemented with antibiotics. We then sampled 50 μl of rearing water and performed cultured-based and 16S-based PCR tests, assessing for the sterility of the treated eggs 24 h post-treatment (FIG. 16).

Germ-free eggs hatched spontaneously post-fertilization (dpf), similarly to non-treated conventional (Conv) eggs, indicating that our sterilization protocol did not affect egg viability. On the contrary, we determined that egg sterilization positively affected hatching efficiency with 72±5.54% for treated eggs and 48.6±6.2% for conventional eggs. Once hatched, a maximum of 12 larvae were transferred into 75 cm³ vented-cap cell culture flasks containing fresh sterile water without antibiotics (FIG. 17). Flask water was renewed every 48 h and GF and Conv fish relied on their vitellus reserves until day 21 days post-hatching (dph), after which they were fed with gamma-ray sterilized fish food powder every 48 h, 30 minutes before water renewal (FIG. 17). In the case of GF fish, we sampled 50 μl of fish water before each water change and one larvae every week to performed culture-based and 16S-based PCR sterility tests until the end of the experiment (35 day dph).

### 35 Dph Germ-Free Trout Show Normal Development and Growth Compared to Conventional Larvae.

To test the consequence of raising GF larvae in sterile conditions, we compared growth performance of Conv and GF larvae reared from the same egg batches and observed no significant differences in standard body length and weight at 35 dph, with 2.33±0.20 cm Vs 2.16±0.11 cm and 0.72±0.21 g vs 0.64±0.19 g for conventional and GF larvae, respectively (FIG. 18).

Consistently, anatomical comparison of Conv and GF trout by optical projection tomography showed no anatomical difference in organ development between conventional or GF fish at 21 dph, even regarding organs in direct contact with fish microbiota such as gills (FIGS. 19D and 19I) and intestine (FIGS. 19C and 19H; FIG. 20), or potentially influenced by gut-microbiota such as brain (FIGS. 19A and 19F), spleen (FIGS. 19B and 19G) or head kidney (FIGS. 19E and 19J) [139]. These results suggest that the natural microbiota have not a major impact on development and growth at this stage of the rainbow trout life-cycle.

### Identification of Fish Pathogens Killing Germ-Free but not Conventional Trout Larvae

To identify pathogens able to infect GF rainbow trout larvae by the natural infection route, we tested several trout bacterial pathogens, including Flavobacterium psychrophilum strain THC-O2/90, F. columnare strain Fc7, Lactococcus garvieae, Vibrio anguillarum strain 1669 and Yersinia ruckeri strain JIP27/88. At 24 dph, GF rainbow trout larvae were exposed 24 h in water containing 10⁷ CFU/mL of the tested pathogen. Fish were then washed 3-times by renewing the 90% of the infection bath by fresh sterile water and then kept at 16°C. under sterile conditions. Among all tested pathogens, F. columnare strain Fc7 was the most virulent pathogen, leading to high and reproducible mortality of GF trout within 48 h after exposure (FIG. 21A and FIG. 25). In contrast to GF trout sensitivity to F. columnare, conventional larvae reared from non-sterilized eggs resisted to infection under all tested conditions (FIG. 21B and FIG. 26). Consistently, histology analysis performed at 25 dph (24 h after infection) showed that infected GF fish displayed dissociation of gill epithelium and a severe disorganization of the intestinal region. In contrast, infected conventional rainbow trout larvae exposed to F. columnare showed no difference with non-infected GF or conventional larvae. More precisely, histological analysis performed at 25 dph (24 h post infection) on GF and Conv larvae did not show any sign of intestinal damage (FIG. 27). However, we observed an increase in goblet cells number in infected vs non-infected GF larvae, whereas Conv infected larvae showed the opposite phenotype when compared to non-infected Conv larvae (FIG. 27).

Conventional Rainbow Trout Microbiota Protects Against F. columnare Infection

Based on the high sensitivity of GF but not conventional rainbow trout to F. columnare Fc7, we hypothesized that observed resistance to infection could be provided by conventional microbiota. To test this, we exposed GF rainbow trout larvae to water used to raised conventional fish at 21 dph, one week before infection challenge by F. columnare Fc7. Re-conventionalized rainbow trout larvae survived to F. columnare equally well than conventional ones, whereas those maintained in sterile conditions rapidly died within the first 24 h after infection (FIG. 22A), suggesting that conventional rainbow trout microbiota provided resistance to F. columnare Fc7 infection. To identify the culturable species within conventional trout microbiota, we plated on various agar growth media dilutions of material extracted from 3 conventional rainbow trout larvae at 35 dph. Individual 16S-based determination of the diversity of bacteria forming colonies on the tested media identified 11 different bacterial strains that were isolated and i stored individually (Table 4).

TABLE 4
The 11 species isolated from conventional rainbow trout larvae
Bacterial species isolated from trout microbiota

Aeromonas rivipollensis
Pseudomonas helmanticensis
Aeromonas rivipollensis
Pseudomonas baetica
Aeromonas hydrophyla
Flavobacterium plurextorum
Acinetobacter sp.
Flavobacterium plurextorum
Delftia acidovorans
Flavobacterium sp.
Pseudomonas sp.

To test whether these 11 culturable strains could contribute to the protection against F. columnare infection observed in Conv trout, we re-conventionalized GF rainbow trout larvae at 22 dph with a mix of all 11 bacterial strains (hereafter called Mix11), each at a concentration of 5×10⁵ CFU/ml. Monitoring the survival of these re-conventionalized trout after exposure to F. columnare strain Fc7 showed that Re-Conv^{Mix11} larvae survived as well as conventional fish (FIG. 22B). These results demonstrated that presence of the 11 bacterial strains isolated from the rainbow trout microbiota recapitulate the full protection against F. columnare infection observed in conventional fish.

Resistance to F. columnare infection is conferred by one member of the trout microbiota To determine whether some individual members of the protective Mix11 could play key roles in infection resistance, we mono-re-conventionalized 22 dph GF trout by each of the 11 isolated bacterial strains at 5.10⁵ CFU/ml followed by challenge with F. columnare Fc7. We found that only Flavobacterium sp. strain 4466 restored Conv-level protection, whereas the other 10 strains displayed no protection, whether added individually (FIG. 23A) or as a mix (Mix10 in FIG. 23B). To evaluate the colonization of gastrointestinal tract by Flavobacterium sp. strain 4466 and/or F. columnare Fc7, we plated intestines from mono-reconventionalized fish 24 hours after infection in TYES agar after dissection in sterile conditions. Interestingly, whereas both Flavobacterium sp. strain 4466 and F. columnare Fc7 were able to successfully colonize the gut of mono-exposed rainbow trout (FIG. 23C), we only detected to Flavobacterium sp. after trout's infection by F. columnare Fc7 (FIG. 23C), suggesting a potential competition between both bacterial species. Consistently, although cell-free spent supernatant of Flavobacterium sp. strain 4466 showed no inhibitory activity against F. columnare Fc7 in an overlay assay (FIG. 28A), Flavobacterium sp. strain 4466 colony growth inhibited the growth of F. columnare Fc7 (FIG. 28B) and of all tested F. columnare strains (FIG. 28C), suggesting a potential contact dependent inhibition. We identified a cluster of 12 genes potentially associated to this phenotype in the Flavobacterium sp. strain 4466 genome (tssB, tssC, tssD, tssE, tssF, tssG, tssH, tssI, tssK, tssN, tssP and tssQ) characteristic of type 6 secretion system (T6SS), T6SSⁱⁱⁱ, a contact-dependent antagonistic system only present in phylum Bacteroidetes [32]. To improve the taxonomic identification of the protective Flavobacterium isolated from the trout larvae microbiota, we performed whole genome sequencing followed by Average Nucleotide Identity (ANI) analysis. We determined that despite similarity with Flavobacterium spartansii (94.65%) and Flavobacterium tructae (94.62%), these values are lower than the 95% ANI needed to identify two organisms as the same species [204]. Furthermore, full-length 16S rRNA and recA genes comparisons also showed high similarity with F. spartansii and F. tructae, however, the obtained values were also below the 99% similarity threshold required to consider that two organisms belong to the same species (See Table at pages 5-6 herein). Similarly, a maximum likelihood based phylogenetic tree (FIG. 29) generated from sequences of 15 bacterial strains from the Flavobacterium genus revealed that the sequence of Flavobacterium sp. strain 4466 clustered with sequences of F. spartansii and F. tructae, but did not allow the identification of Flavobacterium sp. strain 4466 at species level.

Endogenous Flavobacterium sp. Strain 4466 Protects Germ-Free Rainbow Trout Against Infection by Different Strains of F. columnare.

To test whether the protective Flavobacterium sp. isolated from the Conv rainbow trout microbiome could protect rainbow trout we re-conventionalized GF fish larvae with Flavobacterium sp. 48 hours before exposure to four virulent F. columnare strains (Fc7, ALG-00-530, IA-S-4, and Ms-Fc-4) belonging to genomovars I and II, and isolated from different geographical origins and host fish species. Flavobacterium sp. strain 4466 conferred protection to rainbow trout larvae against all F. columnare strains (FIG. 30). Therefore, the Flavobacterium sp. strain identified from trout Mix11 is a putative probiotic potentially protecting trout and other fish from columnaris disease.

Use of Germ-Free Trout to Identify Exogenous Probiotics Against F. columnare Infection.

Our results demonstrated that GF trout could be used as a gnotobiotic models to identify bacteria protecting against F. columnare infection. To determine whether this controlled gnotobiotic approach could be used to identify probiotic beyond bacteria present in trout microbiota, we pre-exposed 22 dph GF rainbow trout larvae to Chryseobacterium massiliae, a bacterium previously shown to protect larval stage and adult zebrafish from infection by F. columnare [Stressmann et al.]. After 48 of balneation with C. massiliae at 10⁶ cfu/mL, we infected trout larvae with four strains of F. columnare: Fc7, ALG-00-530, IA-S-4, and Ms-Fc-4, belonging to the genomovars I and II, and isolated from different geographical origins and host (Table 2). As previously observed in zebrafish axenic model. C. massiliae protects also rainbow trout larvae against F. columnare infection (FIG. 24). Taken together, these results showed that GF rainbow trout enable to rationally identified bacterial species, endogenous or not to trout, with probiotic potential against highly virulent Flavobacterium pathogens.

### Bacterial Taxonomic Identification of the Protective Microorganisms

To taxonomically identify the protective Chryseobacterium sp. and Flavobacterium sp. strain 4466, isolated from the zebrafish and trout larvae microbiota, we performed whole genome sequencing followed by Average Nucleotide Identity (ANI) analysis. The morphotype corresponding to Chryseobacterium sp. was identified at species level as Chryseobacterium massiliae, with a whole-genome similarity of 95,85% (See Tables pages 5 to 7 of present description). Concerning Flavobacterium sp. strain 4466, we determined that despite similarity with Flavobacterium spartansii (94.65%) and Flavobacterium tructae (94.62%), these values are lower than the 95% ANI needed to identify two organisms as the same species [204]. Furthermore, full-length 16S rRNA and recA genes comparisons also showed high similarity with F. spartansii and F. tructae, however, the obtained values were also below the 99% similarity threshold required to consider that two organisms belong to the same species (See Tables pages 5 to 7 of present description).

### Antimicrobial Genes Prediction

Antimicrobial resistance (AMR) gene(s) were found using AMRFinderPlus from the full sequenced genome of each strain. This tool is documented in Feldgarden, M., Brover, V., Haft, D. H., Prasad, A. B., Slotta, D. J., Tolstoy, I., Tyson, G. H., Zhao, S., Hsu, C.-H., McDermott, P. F., Tadesse, D. A., Morales, C., Simmons, M., Tillman, G., Wasilenko, J., Folster, J. P., Klimke, W., 2019. Validating the NCBI AMRFinder Tool and Resistance Gene Database Using Antimicrobial Resistance Genotype-Phenotype Correlations in a Collection of NARMS Isolates. Antimicrob. Agents Chemother. 63 no. 11 (Nov. 1, 2019): e00483-19 https://doi.org/10.1128/AAC.00483-19, so that its implementation is available to the skilled person.

No evidence of AMR genes was detected in the genome of Chryseobacterium sp.

The Flavobacterium sp. 4466 strain chromosome revealed genes encoding resistance to carbapenem, lincosamide, streptogramin, pleuromutilin, and fluoroquinolone antibiotics (Table 6).

TABLE 6
Antimicrobial Resistance genes identification.
Antimicrobial resistance (AMR) gene(s) were found using AMRFinderPlus,
using either protein annotations or nucleotide sequence from the
whole-genome of Flavobacterium sp. strain 4466.

| | | | ARO (Best | Identity |
|---|---|---|---|---|
| Drug class | Mechanism | AMR Gene Family | hit) | (%) |
| Carbapenem | inactivation | IND beta-lactamase | IND-6 | 100 |
| Lincosamide | efflux | ATP-binding cassette (ABC) | IsaA | 100 |
| antibiotic; | | antibiotic efflux pump | | |
| streptogramin | | | | |
| antibiotic; | | | | |
| pleuromutilin | | | | |
| antibiotic | | | | |
| Carbapenem; | inactivation | JOHN beta-lactamase | JOHN-1 | 78.5 |
| cephalosporin; | | | | |
| penam | | | | |
| Fluoroquinolone | efflux | resistance-nodulation-cell | adeF | 42.3 |
| antibiotic; | | division (RND) antibiotic | | |
| tetracycline | | efflux pump | | |
| antibiotic | | | | |
| Fluoroquinolone | efflux | resistance-nodulation-cell | adeF | 42.5 |
| antibiotic; | | division (RND) antibiotic | | |
| tetracycline | | efflux pump | | |
| antibiotic | | | | |

### Virulence Genes Prediction

The isolated strain Chryseobacterium sp. contained five predicted virulence factors, including some proteins involved in capsule biosynthesis, heat shock protein HtpB subunit, KatA catalase and ClpP protease proteolytic subunit (Table 7).

Table 7. Virulence genes identification for Chryseobacterium massiliae. Virulence factor coding gene(s) were predicted using the Virulence Factors Database, using either protein annotations or nucleotide sequence from the whole-genome of Chryseobacterium massiliae.

For Flavobacterium sp. 4466, genes encoding for capsule, sialic acid synthase, type IV and type VI secretion systems effectors and catalase were found as potential virulence factors (Table 8).

TABLE 8
Virulence genes identification for Flavobacterium sp. strain 4466.
Virulence factor coding genes were predicted using the Virulence Factors Database,
using either protein annotations or nucleotide sequence from the
whole-genome of Flavobacterium sp strain 4466.

| | | | Query |
|---|---|---|---|
| Description | E-value | Identity (%) | coverage (%) |
| UDP-galactopyranose mutase [Capsule] (cpsl) | 2.420e−40 | 64.41 | 94.52 |
| UDP-galactopyranose mutase [Capsule] (glf) | 2.580e−84 | 67.36 | 93.98 |
| Sialic acid synthase (neuB1) | 5.670e−86 | 67.76 | 92.8 |
| Hsp60, 60K heat shock protein HtpB [Hsp60] | 1.160e−147 | 68.07 | 92.51 |
| (htpB) | | | |
| Coxiella Dot/Icm type IVB secretion system | 3.380e−45 | 65.19 | 75.9 |
| translocated effector, trans-2-enoyl-CoA | | | |
| reductase [T4SS effectors] | | | |
| UDP-N-acetylglucosamine 2-epimerase (neuC1) | 4.740e−43 | 66.55 | 74.11 |
| GDP-mannose 4,6-dehydratase [O-antigen] | 3.460e−114 | 71.54 | 73.74 |
| (gmd) | | | |
| GDP-D-mannose dehydratase [Capsule] (gmd) | 4.510e−62 | 67.63 | 70.3 |
| Catalase-peroxidase KatB [KatAB (VF0168)] | 9.350e−81 | 6537 | 65.94 |
| (katB) | | | |
| Polysialic acid capsule biosynthesis protein SiaC | 6.920e−66 | 69 | 65.03 |
| [Capsule] (siaC/synC) | | | |
| Catalase/(hydro)peroxidase [KatAB] (katA) | 2.860e−68 | 64.93 | 61.18 |
| Vi polysaccharide biosynthesis protein, UDP- | 4.260e−57 | 67.28 | 54.03 |
| glucose/GDP-mannose dehydrogenase TviB | | | |
| (tviB) | | | |

### Discussion

Many studies focus on the effects of microbial diversity on the properties of higher-order bacterial community. In this study, we evidenced a novel community-level protective effect of the resident microbiota against deadly infection. More specifically, we used re-conventionalization of otherwise germ-free zebrafish larvae to show that conventional-level protection against infection by a broad range of highly virulent F. columnare strains is provided by a set of 10 culturable bacterial strains belonging to 9 different species from standard laboratory zebrafish microbiota. With the exception of the Bacteroidetes C. massiliae, this protective consortium was dominated by Proteobacteria such as Pseudomonas and Aeromonas spp. and by bacteria commonly found in aquatic environments. Despite the relative permissiveness of zebrafish larvae microbiota to environmental variations and inherent variability between samples [36], we showed that these ten bacteria were also identified as predominant in four different zebrafish facilities, suggesting the existence of a core microbiota with important functionality.

Use of germ-free and gnotobiotic zebrafish larvae exposed to controlled combinations of bacterial species enabled us to show a very robust species-specific protection effect in larvae mono-associated with C. massiliae. We also identified a community-level protection provided by the combination of the 9 other species composing the protective zebrafish larvae microbiota that were otherwise unable to protect against F.columnare when provided individually. This protection was however less reproducible (full protection seen in only 50% of the tests performed) and required a minimum inoculum of 5.10⁴ cfu/mL, compared to 5.10² cfu/mL with C. massiliae. These results therefore suggest the existence of two distinct microbiota-based protection scenarios against infection by F. columnare, a membership effect provided by C. massiliae, and a threshold effect mediated by the Mix9 consortium.

Neither of these two protection mechanisms against F. columnare infection seem to rely on microbiota-based immuno-modulation. However, we cannot exclude that, individually, some members of the protective Mix10 could induce, pro- or anti-inflammatory responses masked in presence of the mixed microbiota [1]. Whereas the identification of mechanisms involved in the community-level Mix9 protection will require further studies, re-conventionalization and dysbiosis and recovery experiments demonstrated the key role of C. massiliae in resistance against F. columnare. This protection could be provided by a number of mechanisms, including nutrient depletion or competition, adhesion inhibition, release of inhibitory metabolites and stimulation of host immune defenses [6, 12, 37]. [37]. As Mix9 and C. massiliae showed a difference in minimum cell density required for protection, it is also possible that for the latter, F. columnare infection triggers a relatively density-independent protection mechanism in C. massiliae by direct antagonistic niche-exclusion. F. columnare and C. massiliae are indeed both bacteroidetes, and, besides direct resource competition, several mechanisms of niche exclusion were shown to occur between phylogenetically close Bacteroidetes species, including toxin production [38, 39] or toxin-injection dependent on the Type 6 secretion system [40]. Experiments are currently underway to identify non-protective C. massiliae mutants to further analyze its protective mechanism. Interestingly, infected larvae re-conventionalized with either C. massiliae or Mix9 showed no signs of the intestinal damage displayed by germ-free larvae, suggesting that both C. massiliae and Mix9 provide similar intestinal resistance to F. columnare infection. Whereas microbial colonization contributes to gut maturation and stimulates the production of epithelial passive defenses such as mucus [41, 42], lack of intestinal maturation is unlikely to be contributing to F. columnare-induced mortality, as mono-colonized larvae or larvae re-conventionalized with non-protective mixes died as rapidly as GF larvae.

Several studies have monitored the long-term assembly and development of the zebrafish microbiota from larvae to sexually mature adults, however little is known about the initial colonization of the larvae after hatching [43, 44]. Neutral (stochastic) and deterministic (host niche-based) processes [45-47] lead to microbial communities that are mostly represented by a limited number of highly abundant species with highly diverse low-abundant populations. In our experiments, the Mix10 species inoculum corresponded to an equiratio bacterial mix, thus starting from assumed total evenness (E=1) [48, 49]. Since evenness was still relatively high (0.84) and remained very similar up until 8h in our study, this indicated that most of the ten species were able to colonize the larvae. From the perspective of community composition, a loss of diversity is often associated with decreased colonization resistance, but it remains unclear whether this increased susceptibility is due to the loss of certain key member species of the microbial community and/or a change in their prevalence [7, 8]. We investigated resistance to infection by exposing established bacterial communities to different antibiotic perturbations, followed by direct challenge with F. columnare (to study core microbiota sensitivity to disturbance) or after recovery (to study its resilience) [11, 50]. Antibiotics are known to shift the composition and relative abundances of the microbiota according to their spectrum [12, 51]. We observed that penicillin/streptomycin treatment that would affect most of the core species reduced the abundance of all but two species (A. veronii 1 and P. myrsinacearum) that became relatively dominant during recovery but failed to provide protection against F. columnare. With the kanamycin treatment, colonization resistance was fully restored at the end of the recovery period, indicative of a resilience that could result from species recovering quickly to their pre-perturbation levels due to fast growth rates, physiological flexibility or mutations [52]. Interestingly, even taking into account potential biases associated with the use of the 16S rDNA as a proxy index to determine relative abundance [53, 54], evenness was similarly reduced during recovery for both treatments, but abundance at phylum level changed to 48% for Proteobacteria, and 52% for Bacteroidetes compared to the >98% of Proteobacteria with the penicillin/streptomycin treatment. Furthermore, C. massiliae was detected as rare (<1%) in conventional larvae, suggesting that it could have a disproportionate effect on the community or that community-level protection provided by the nine other bacteria was also responsible for the protection of conventional larvae to F. columnare infection.

Despite the phenotypic homogeneity of symptoms associated with columnaris disease caused by F. columnare on cold and warm water fish, F. columnare strains show high genetic diversity making infection animal models difficult to standardize [34, 55, 56]. Our studies showed that germ-free zebrafish larvae are highly susceptible to a variety of different genomovars isolated from different hosts, demonstrating that they are a robust animal model for the study of F. columnare pathogenicity. Although F. columnare infection causes important losses in aquaculture, there is no consensus on the molecular bases of F. columnare virulence. Secreted enzymes such as chondroitin AC lyase acting on connective tissue [33, 57-59], and collagenase [60] have been suggested as possible virulence factors [31]. Recently, F. columnare mutants in Type 9 secretion system (T9SS) were shown to be avirulent in adult zebrafish, suggesting that proteins secreted by the T9SS are likely to be key for virulence [33]. The colonization process of F. columnare also remains largely unidentified [31]. In salmonid fish, the gills are major sites of infection, but body skin, fins and tail are also frequently damaged, and septicemia can occur in severe cases [57]. In salmon, gross tissue damage of several organs was associated with low virulent strains, whereas highly virulent strains killed before such damage was seen [31]. We could not identify clear F. columnare infection sites in zebrafish larvae by histology, perhaps due to its very low dose of infection, with less than 100 cfu recovered from infected moribund larvae. However, several lines of evidence suggest that the gut is the main target of F. columnare infection in our model: (i) unfed GF larvae survived exposure, (ii) histology analysis showing severe disruption of the intestinal region just hours after infection in GF larvae, and (iii) induction of il22 in GF larvae exposed to F. columnare, since a major function of IL-22 is to promote gut repair [61]. This induction appears to be a consequence of the pathogen-mediated damage, as there was no observed induction in conventional or re-conventionalized larvae. The very rapid death of larvae likely caused by this severe intestinal damage probably explains why little damage could be observed in other common target organs of columnaris such as the gills, skin and fins.

Many bacterial diseases affect aquaculture, and cannot be controlled by vaccination because they affect young fry or because efficient vaccines are not available (for example, for flavobacterioses). In these cases, antibiotic use remain the only option, potentially resulting in spread of antibiotic resistance. There is an urgent need for the development of alternative treatments [11]. In the case of F. columnare infections, both high genetic variability and broad host range constitute an important limitation for the identification of effective probiotics against this widespread pathogen. In this study, we showed that C. massiliae could be a promising probiotic candidate to prevent columnaris diseases as it provided full and robust protection against all tested virulent F. columnare genomovars and was also able to significantly increased survival of adult conventional zebrafish exposed to this pathogen. Whereas further studies are needed to elucidate C. massiliae protection potential in other teleost fish, the endogenous nature of C. massiliae suggest that it could establish itself as a long-term resident of the zebrafish larval and adult microbiota, an advantageous trait when seeking probiotic adaptability to targeted fish species [62]. While short-term residing probiotics potentially limit unintended consequences to the microbial community and host system, use of endogenous residents can stably modulate the community and protect the fish over long periods against reoccurring disease outbreaks [63].

In conclusion, the use of a simple and tractable fish model to mine indigenous fish microbiota as a source of protection against a fish pathogen further underlines the power of the zebrafish model for analysis of microbiota function. Our study contributes to the expansion of knowledge on microbiota-mediated colonization and infection resistance against an important fish pathogen. Further study will determine the potential of endogenous bacteria as aquaculture probiotics to improve the health and production of other teleost fish.

Use of probiotics to improve fish growth performance and health and limit the use of chemical and antibiotic treatment is now a common approach to control disease outbreaks in fish farming industry [123, 140, 141]. However, identification and characterization of protective bacteria are hampered by experimental variability associated with administration and infection challenges performed in open environmental conditions using poorly controlled conventional fish. The development of robust and reproducible gnotobiotic models are therefore instrumental to develop fish probiotics [125, 131]. Here, we established a new model of germ-free and gnotobiotic model of rainbow trout, enabling the controlled study of probiotic-based protection against infection caused by several fish bacterial pathogens.

The resistance of rainbow trout eggs to the effective disinfection protocol eliminated the microbial community associated to the egg surface, enabling routinely raise of germ-free larvae for up to 35 dph at 16° ° C. without continued exposure to antibiotics. Our protocol is therefore comparable to gnotobiotic protocols used for zebrafish [136, 142], cod larvae [128], and stickleback (Gasterosteus aculeatus) that are not dependent on continued addition of antibiotics, hence avoiding possible long-term effects on fish development [131]. Further, after hatching, fish larvae were immediately transferred to cell culture flasks with vented caps. Fish husbandry in flasks presents some disadvantages that hinder long-term experiments such as the no way to aerate water and to perform water changes automatically [131]. These constraints limit this model as an effective method for short-term experiments. The relative short-term of the experiments performed to investigate infection resistance in trout larvae, while effective to control sterility, present the disadvantage to work with larvae with low complexity microbiota. Axenic and gnotobiotic conditions are artificial compared to the conventional conditions of larval rearing, in both fish farming or wild-life [128]. However, even if adding a bacterial strain as pure culture may not be representative of the effect of natural host-associated microbiota, this model is an excellent available tool for the study of the effects of specific bacterial additives, without any microbial interference.

Raising under germ-free conditions has no major impact on development and growth of rainbow trout larvae at 21dph. Similar results were reported for germ-free stickleback larvae at 14 dph [143]. In sea bass (D. labrax L.) germ-free raised larvae reported higher growth and more developed gut compared to conventionally raised larvae [144]. This controversy could come from the fact that in our study and in GF stickleback, anatomical analysis was performed before the first-feeding, whereas germ-free sea bass were externally fed. Fish initially acquire nutrients by absorbing their endogenous yolk until the intestinal track is open from the mouth to the vent. We cannot rule out that, at later developmental stage or after fish first-feeding differences may occur in the global body weight as well as in the structure and size of organs such as gut between GF and conventional fish.

Salmonids, including rainbow trout, are commercially important species, which production in intensive aquaculture facilities is associated with increased susceptibility to diseases caused by viruses, bacteria, fungi and parasites [145]. Here, we tested the sensitivity of germ-free and conventional trout larvae to major salmonid freshwater pathogens. This led to identify F. columnare as a highly virulent species, lethal for GF larvae. F. columnare, is the causative agent of columnaris diseases, affecting several aquaculture fish species [137, 146] and an emerging problem for larval and juvenile rainbow trout [147, 148]. By contrast with the high sensitivity displayed by germ-free trout, conventional larvae reared from non-sterilized eggs were fully resistant to F. columnare infection and we showed that commensal microbiota harbored by conventional trout larvae plays an essential role in protecting against F. columnare.

While GF conditions cannot be compared to those prevailing in the wild or used in fish farming [205], our results showed that GF rainbow trout larvae are highly susceptible to F. columnare, the causative agent of columnaris disease affecting many aquaculture fish species [206,207]. Although our histology analysis comparing GF and Conv larvae infected or not by F. columnare Fc7 did not show any major sign of inflammation of damage, we observed that the number of Goblet cells per crypt increases in infected GF larvae and decreased in Conv larvae. A healthy intestine is determined by biological markers such as Goblet cells count, which secrete mucus with bactericidal properties [208]. Interestingly, a significative decrease in the number of Goblet cells was also observed of non-infected GF larvae compared to those of Conv larvae, as previously reported in zebrafish [209]. The absence of stimulating microorganisms in GF larvae could lead to a dysregulated acute immune response after F. columnare infection. These results suggest that the microbiota influence cell differentiation (or maturation) in trout gut epithelium [210], potentially affecting for some aspect of the protection against F. columnare infection.

Different studies have demonstrated that high diverse gut communities exert higher protection on the host [149-151]. This constitute the bases for the paradoxical negative effect on fish health associated with the massive utilization of antibiotics in aquaculture, which, by reducing microbiota diversity in turn, facilitates colonization by opportunistic pathogens [152].

Whereas this advocates for practices leading to enrichment of fish microbial community to minimize pathogenic invasions in aquaculture [122], our results demonstrates that resistance to infection can be achieved by the relatively low complexity of the culturable microbiota identified in conventional trout larvae. We indeed only identified 11 different bacterial species from conventional rainbow trout larvae microbiota, nevertheless conferring full protection to re-conventionalized GF trout. Whereas example of resistance to infection provided by controlled bacterial consortia in gnotobiotic host often rely more on community structure than on individual members of the microbiota [153-156], we showed that the protection observed for the bacterial consortium composed of the 11 identified microorganisms is mainly due to the presence of Flavobacterium sp strain 4466. We cannot however, rule out that, at later developmental stages, the presence of other bacterial species may be needed for more efficient implantation or stability of protective members in the trout microbiota.

For the past 30 years, the fish farming industry dedicated a considerable amount of efforts to identify probiotic microorganisms for rainbow trout, including Gram-positive and Gram-negative bacteria and yeast [157]. However, the irreproducibility of many of the in vivo experimentations, the high interindividual and seasonal variability of trout microbiota composition and the random of limited colonization ability of exogenous microorganism rarely enable to firmly establish probiotic properties [158-160]. Identification of Flavobacterium sp., an indigenous member of trout larvae microbiota protecting against F. columnare infection suggested that such bacteria could be used as probiotics to prevent infections. Although there is yet no clear evidence that probiotics of indigenous origin would perform better than probiotic exogenous to the target host [161], the use of beneficial indigenous bacteria isolated from aquatic organisms is gaining recognition for controlling pathogens within the aquaculture industry [162].

The high genetic variability of F. columnare, and its broad host range constitute an important limitation for the identification of effective probiotics against this widespread pathogen. Several probiotic candidates isolated from the host provided partial protection against F. columnare infection in other conventional fish species such as walleye (Sander vitreous) and brook charr (Salvelinus fontinalis) [163, 164]. However, a high protection variability was observed for probiotic strains used on brook charr challenged with F. columnare, depending on the fish family used. As authors suggested, based on that microbiota composition is directly influenced by the host genotype, each family genetic background controls the efficiency of probiotic effect on the pathogen [163]. Using germ-free or gnotobiotic animal models, as proposed in this study, should decrease this variability for a more precise evaluation of probiotic candidate strains. According to the different criteria defined for bacterial probiotic selection [157, 161], our study suggests that Flavobacterium sp., could be considered as promising endogenous probiotics, which potential in aquaculture needs to be further established on different stages of trout life cycle.

More precisely, inventors have in particular demonstrated the ability of Flavobacterium sp. strain 4466 isolated from Conv trout larvae microbiota to protect against F. columnare infection. Furthermore, this bacterium, but not its supernatant, inhibits F. columnare growth in vitro, which suggests a direct interaction between Flavobacterium sp. strain 4466 and F. columnare. Intriguingly, Flavobacterium sp. strain 4466 encodes a complete subtype T6SSⁱⁱ, a molecular mechanism that delivers antimicrobial effector proteins upon contact with target cells and is unique to the phylum Bacteroidetes [211]. The members of Flavobacterium genus are ubiquitous inhabitants of freshwater and marine fish microbiota and both commensal and pathogenic Flavobacterium often share the same ecological niche [212-214]. Whether the Flavobacterium sp. strain 4466 T6SSⁱⁱⁱ contact-dependent killing system contributes to colonization resistance by inhibiting F. columnare Fc7 growth is currently under investigation. We cannot, however, exclude other mechanisms such as competition for nutrients or pathogen exclusion upon direct competition for adhesion to host tissues. This process has been suggested for infected zebrafish with efficient colonization of highly adhesive probiotic strains and enhanced life expectancy [215,216,217].

Interestingly, our germ-free rainbow trout larvae model also allowed us to demonstrate the protective activity of Chryseobacterium massiliae, a potential probiotic bacterium isolated from conventional zebrafish [Stressman], against different strains of F. columnare from different host and geographical origins. These results support C. massiliae as a potential probiotic to prevent columnaris diseases in other teleost fish apart from its original host, zebrafish. Further, this germ-free fish model shows a wide range of possibility for the study of endogenous and exogenous potential probiotic strains against infections.

In conclusion, by using experimental conditions reducing microbiota variability, germ free rainbow trout larvae allow to perform a challenges under gnotobiotic conditions, and lead to clear analysis of protection phenotypes against fish pathogens. This approach will also be instrumental in studying the host-pathogen interaction under controlled conditions to better understand the virulence mechanisms used by fish pathogens. Altogether, this model could contribute to mitigate rainbow trout fish diseases in the context of aquaculture research and husbandry.

### Material and Methods

Bacterial strains and growth conditions. Bacterial strains used in this study are listed in Table 1. F. columnare strains (Table 5) were grown at 28° C. in tryptone yeast extract salts (TYES) broth [0.4% (w/V) tryptone, 0.04% yeast extract, 0.05% (w/v) MgSO₄ 7H₂O, 0.02% (w/V) CaCl₂) 2H₂O, 0.05% (w/V) D-glucose, pH 7.2]. F. columnare were assigned into four genomovar groups using 16S rDNA restriction fragment length polymorphism analysis, including genomovar I, I/II, II, and III[64]. All 10 Mix10 microbiota species were grown in Luria Bertani (LB) at 28° C.

TABLE 5
Flavobacterium columnare strains used in this study

| | | |
|---|---|---|
| | | Source or |
| Strain | Description | Reference |

F. columnare strains

| | | |
|---|---|---|
| FPC666 | F. columnare isolated from Misgurnus anguillicaudatus (Japan). | J.F. Bernardet col. |
| FPC667 | F. columnare isolated from Carassius auratus (Japan). | J.F. Bernardet col. |
| LD40 07/2489 | F. columnare isolated from Acipenser baeri (France). | J.F. Bernardet col. |
| LVDI 39/1 | UNKNOWN | J.F. Bernardet col. |
| JIP 17/01 | F. columnare isolated from Cyprinus carpio (France). | J.F. Bernardet col. |
| JIP P06/90 | F. columnare isolated from Ictalurus melas (France). | J.F. Bernardet col. |
| LVDL 3414/89 | F. columnare isolated from Anguilla anguilla (France). | J.F. Bernardet col. |
| UJ H2 | F. columnare isolated from Oncorhynchus mykiss (Finland). | J.F. Bernardet col. |
| UJ B259 | F. columnare isolated from outlet water of a rearing tank with | J.F. Bernardet col. |
| | infected Oncorhynchus mykiss (Finland). | |
| Fc4 | F. columnare isolated from Oncorhynchus mykiss (USA). | J.F. Bernardet col. |
| Fc7 | F. columnare isolated from Oncorhynchus mykiss (USA). | J.F. Bernardet col. |
| JIP P11/91 | F. columnare isolated from Oncorhynchus mykiss (France). | J.F. Bernardet col. |
| JIP 44/00 | | J.F. Bernardet col. |
| NCIMB2248 | F. columnare isolated from Oncorhynchus tshawytscha (USA). | J.F. Bernardet col. |
| JIP 39/87 | F. columnare isolated from Ictalurus punctatus (France). | J.F. Bernardet col. |
| JIP 07/02(1) | F. columnare isolated from Cyprinus carpio (France). | J.F. Bernardet col. |
| ALG-00-530 | F. columnare isolated from Ictalurus punctatus (USA). | {Olivares-Fuster, |
| | | 2011 #101} |
| JIP 14/00 | F. columnare isolated from Paracheirodon innesi (France). | J.F. Bernardet col. |
| EK28 | F. columnare isolated from Anguilla japonica (Japan). | J.F. Bernardet col. |
| AJSI | F. columnare isolated from Poecilia sphenops (Belgium). | J.F. Bernardet col. |
| JIP 13/00 | F. columnare isolated from Paracheirodon innesi (Hong Kong). | J.F. Bernardet col. |
| JIP 02/06(1) | F. columnare isolated from Betta splendens (Singapore). | J.F. Bernardet col. |
| VB1 | F. columnare isolated from Poecilia reticulata (France). | J.F. Bernardet col. |
| VB2 | F. columnare isolated from Poecilia reticulata (France). | J.F. Bernardet col. |
| LDA39 H4927 | F. columnare isolated from Ictalurus melas (France). | J.F. Bernardet col. |
| SNARCC90 | F. columnare isolated from Ictalurus punctatus (USA). | J.F. Bernardet col. |
| CIP109753 | F. columnare isolated from Plecoglossus altivelis (Japan). | CRBIP |
| C#2 | F. columnare isolated from Pelteobagrus fulvidraco (Unknown). | {Li N. et al., 2017} |
| FCC-2 | ΔgldN in strain C#2 | {Li N. et al., 2017} |
| FCC-8 | ΔporV in strain C#2 | {Li N. et al., 2017} |
| FCC-3 | FCC-2 + pLN8 for gldN complementation | {Li N. et al., 2017} |
| FCC-9 | FCC-8 + pLN11 for porV complementation | {Li N. et al., 2017} |

### Ethics Statement

All animal experiments described in the present study were conducted at the Institut Pasteur (larvae) or at INRA Jouy-en-josas (adults) according to European Union guidelines for handling of laboratory animals (http://ec.europa.eu/environment/chemicals/lab_animals/home_en.htm) and were approved by the relevant institutional Animal Health and Care Committees.

General handling of zebrafish. Wild-type AB fish, originally purchased from the Zebrafish International Resource Center (Eugene, OR, USA), or myd88-null mutants (myd88^{hu3568/hu3568} [35], kindly provided by A. H Meijer (Leiden University, the Netherlands), were raised in our facility. A few hours after spawning, eggs were collected, rinsed, and sorted under a dissecting scope to remove feces and unfertilized eggs. All following procedures were performed in a laminar microbiological cabinet with single-use disposable plasticware. Fish were kept in sterile 25 cm³ vented cap culture flasks containing 20 ml of water (0-6 dpf-15 fish per flasks) or 24-well microtiter plates (6-15 dpf-1 fish per 2 mL well) in autoclaved mineral water (Volvic) at 28° C. Fish were fed 3 times a week from 4 dpf with germ-free Tetrahymena thermophila protozoans [23]. Germ-free zebrafish were produced after sterilizing the egg chorion protecting the otherwise sterile egg, with antibiotic and chemical treatments (see below), whereas conventional larvae (with facility-innate microbiota) were directly reared from non-sterilized eggs and then handled exactly as the germ-free larvae.

Sterilization of zebrafish eggs. Egg sterilization was performed as previously described with some modifications [23]. Freshly fertilized zebrafish eggs were first bleached (0.003%) for 5 min, washed 3 times in sterile water under gentle agitation and maintained overnight groups of 100 eggs per 75 cm³ culture flasks with vented caps containing 100 mL of autoclaved Volvic mineral water supplemented with methylene blue solution (0,3 μg/mL). Afterwards, eggs were transferred into 50 mL Falcon tubes (100 eggs per tube) and treated with a mixture of antibiotics (500 μL of penicillin G: streptomycin, 10,000 U/ml: 10 mg/mL GIBCO #P4333), 200 μL of filtered kanamycin sulfate (100 mg/mL, SERVA Electrophoresis #26899) and antifungal drug (50 μL of amphotericin B solution Sigma-Aldrich (250 μg/mL) #A2942) for 2 h under agitation at 28° C. Eggs were then washed 3 times in water under gentle agitation and bleached (0.003%) for 15 min, resuspending the eggs every 3 min by inversion. Eggs were washed again 3 times in water and incubated 10 min with 0,01% Romeiod (COFA, Coopérative Française de l'Aquaculture). Finally, eggs were washed 3 times in water and transferred into 25 cm³ culture flasks with vented caps containing 20 ml of water. After sterilization, eggs were transferred with approximately 30 to 35 eggs/flasks, and were transferred into new flasks at 4 dpf before reconventionalization with 10 to 15 fish /flask. We monitored sterility at several points during the experiment by spotting 50 μL of water from each flask on LB, TYES and on YPD agar plates, all incubated at 28° C. under aerobic conditions. Plates were left for at least 3 days to allow slow-growing organisms to multiply. Spot checks for bacterial contamination were also carried out by PCR amplification of water samples with the 16S rDNA gene primers and procedure detailed further below. If a particular flask was contaminated, those fish were removed from the experiment.

Procedure for raising germ-free zebrafish. After hatching, fish were fed with germ-free T. thermophila 3 times per week from 4 dpf onwards. (i) T. thermophila stock. A germ-free line of T. thermophila was maintained at 28° ° C. in 20 mL of PPYE (0.25% protease peptone BD Bact #211684, 0.25% yeast extract BD Bacto #212750) supplemented with penicillin G (10 unit/mL) and streptomycin (10 μg/mL). Medium was inoculated with 100 μl of the preceding T. thermophila stock. After one week of growth, samples were taken, tested for sterility on LB, TYES and YPD plates and restocked again. (ii) Growth. T. thermophila were incubated at 28° C. in MYE broth (1% milk powder, 1% yeast extract) inoculated from stock suspension at a 1:50 ratio. After 24 h of growth, Tetrahymena were transferred to Falcon tubes and washed (4400 rpm, 3 min at 25° C.) 3 times in 50 mL of autoclaved Volvic water. Finally. T. thermophila were resuspended in water and added to culture flasks (500 μL in 20 mL) or 24-well plates (50 μL/well). Sterility of T. thermophila was tested by plating and 16S rDNA PCR as described in the section above. (iii) Fine-powder feeding. When indicated, fish were fed with previously [symbol] -ray-sterilized fine-powdered food suitable for an early first feeding gape size (ZM-000 fish feed, ZM Ltd) every 48 hours [65].

Reconventionalization of germ-free zebrafish. At 4 dpf, just after hatching, zebrafish larvae were reconventionalized with a single bacterial population or a mix of several. The 10 bacterial species constituting the core protective microbiota were grown for 24 h in suitable media (TYES or LB) at 28° C. Bacteria were then pelleted and washed twice in sterile water, and all adjusted to the same cell density (OD₆₀₀=1 or 5.10⁷ cfu/mL) (i) Reconventionalization with individual species. Bacteria were resuspended and transferred to culture flasks containing germ-free fish at a final concentration of 5.10⁵ cfu/mL. (ii) Reconventionalization with bacterial mixtures. For the preparation of Mix10, Mix9, Mix8 and all other mixes used, equimolar mixtures were prepared by adding each bacterial species at initial concentration to 5.10⁷ cfu/mL. Each bacterial mixture suspension was added to culture flasks containing germ-free fish at a final concentration of 5×10⁵ cfu/mL.

Infection challenges. F. columnare strains (Table 5) were grown overnight in TYES broth at 28° C. Then, 2 mL of culture were pelleted (10,000 rpm for 5 min) and washed once in sterile water. GF zebrafish were brought in contact with the tested pathogens at 6 dpf for 3 h by immersion in culture flasks with bacterial doses ranging from 5.10² to 5.10⁷ cfu/mL. Fish were then transferred to individual wells of 24-well plates, containing 2 ml of water and 50 UL of freshly prepared GF T. thermophila per well. Mortality was monitored daily as described in and as few as 54±9 cfu/larva of F. columnare were recovered from infected larvae. All zebrafish experiments were stopped at day 9 post-infection and zebrafish were euthanized with tricaïne (MS-222) (Sigma-Aldrich #E10521). Each experiment was repeated at least 3 times and between 10 and 15 larvae were used per condition and per experiment.

Collection of Eggs from Other Zebrafish Facilities

Conventional zebrafish eggs were collected in 50 mL Falcon tubes from the following facilities: Facility 1: Nadia Soussi-Yanicostas facility in Hopital Robert Debré, Paris; Facility 2: Jussieu A2, University Paris 6; Facility 3: Jussieu—C8 (UMR7622), University Paris 6; Facility 4: AMAGEN commercial facility, Gif sur Yvette; Larvae were treated with the same rearing conditions, sterilization and infection procedures used in the Institut Pasteur facility.

Determination of fish bacterial load using cfu count. Zebrafish were euthanized with tricaine (MS-222) (Sigma-Aldrich #E10521) at 0.3 mg/mL for 10 minutes. Then they were washed in 3 different baths of sterile PBS-0.1% Tween to remove bacteria loosely attached to the skin. Finally, they were transferred to tubes containing calibrated glass beads (acid-washed, 425 μm to 600 μm, SIGMA-ALDRICH #G8772) and 500 μL of autoclaved PBS. They were homogenized using FastPrep Cell Disrupter (BIO101/FP120 QBioGene) for 45 s at maximum speed (6.5 m/s). Finally, serial dilutions of recovered suspension were spotted on TYES agar and cfu were counted after 48 h of incubation at 28° C.

Characterization of zebrafish microbial content. Over 3 months, the experiment was run independently 3 times and 3 different batches of eggs were collected from different fish couples in different tanks. Larvae were reared as described above. GF and Conv larvae were collected at 4 dpf, 6 dpf and 11 dpf for each batch. Infected Conv larvae were exposed to F.columnnare^{ALG} for 3 h by immersion as described above. For each experimental group, triplicate pools of 10 larvae (one in each experimental batch) were euthanized, washed and lysed as above. Lysates were split into 3 aliquots, one for culture followed by 16S rDNA gene sequencing (A), for 16S rDNA gene clone library generation and Sanger sequencing (B) and for Illumina metabarcoding-based sequencing (C).

A) Bacterial Culture Followed by 16S rDNA Gene-Based Identification

Lysates were serially diluted and immediately plated on R2A, TYES, LB, MacConkey, BHI, BCYE, TCBS and TSB agars and incubated at 28°C. for 24-72h. For each agar, colony morphotypes were documented, and colonies were picked and re-streaked on the same agar in duplicate. In order to identify the individual morphotypes, individual colonies were picked for each identified morphotype from each agar, vortexed in 200 μL DNA-free water and boiled for 20 min at 90ºC. Five μL of this bacterial suspension were used as template for colony PCR to amplify the 16S rDNA gene with the universal primer pair for the Domain bacteria 8f (5′-AGA GTT TGA TCC TGG CTC AG-3′ (SEQ ID NO:7)) and 1492r (5′-GGT TAC CTT GTT ACG ACT T-3′ (SEQ ID NO:8)). Each primer was used at a final concentration of 0.2 μM in 50 μL reactions. PCR cycling conditions were—initial denaturation at 94° C. for 2 min, followed by 32 cycles of denaturation at 94° C. for 1 min, annealing at 56° ° C. for 1 min, and extension at 72° ° C. for 2 min, with a final extension step at 72° C. for 10 min. 16S rDNA gene PCR products were verified on 1% agarose gels, purified with the Qiaquick® PCR purification kit and two PCR products for each morphotype were sent for sequencing (Eurofins, Ebersberg, Germany). 16S rDNA sequences were manually proofread, and sequences of low quality were removed from the analysis. Primer sequences were trimmed, and sequences were compared to GenBank (NCBI) with BLAST, and to the Ribosomal Database Project with SeqMatch. For genus determination a 95% similarity cut-off was used, for Operational Taxonomic Unit determination, a 98% cut-off was used.

B) 16S rDNA Gene Clone Library Generation

Total DNA was extracted from the lysates with the Mobio PowerLyzer® Ultraclean® kit according to manufacturer's instructions. Germ-free larvae and DNA-free water were also extracted as control samples. Extracted genomic DNA was verified by Tris-acetate-EDTA-agarose gel electrophoresis (1%) stained with GelRed and quantified by applying 2.5 μL directly to a NanoDrop® ND-1000 Spectrophotometer. The 16S rDNA gene was amplified by PCR with the primers 8f and 1492r, and products checked and purified as described in section A. Here, we added 25-50 ng of DNA as template to 50 μL reactions. Clone libraries were generated with the pGEM®-T Easy Vector system (Promega) according to manufacturer's instructions. Presence of the cloned insert was confirmed by colony PCR with vector primers gemsp6 (5′-GCT GCG ACT TCA CTA GTG AT-3′ (SEQ ID NO:9)) and gemt7 (5′-GTG GCA GCG GGA ATT CGA T-3′ (SEQ ID NO:10)). Clones with an insert of the correct size were purified as above and sent for sequencing (Eurofins, Ebersberg, Germany). Blanks using DNA-free water as template were run for all procedures as controls. Clone library coverage was calculated with the following formula [1−(n₁/N₂)]×100, where n₁ is the number of singletons detected in the clone library, and N₂ is the total number of clones generated for this sample. Clone libraries were generated to a minimum coverage of 95%, and a minimum of 48 clones was generated for each sample. Sequence analysis and identification was carried out as in section A.

C) By 16S rDNA Gene Illumina Sequencing

To identify the 16S rDNA gene diversity in our facility and fish collected from 4 other zebrafish facilities, fish were reared as described above. GF fish were sterilised as above, and uninfected germ-free and conventional fish were collected at 6 dpf and 11 dpf. Infection was carried out as above with F. columnare^{ALG} for 3h by bath immersion, followed by transfer to clean water. Infected conventional fish were collected at 6 dpf 6h after infection with F. columnare and at 11 dpf the same as uninfected fish. GF infected larvae were only collected at 6 dpf 6h post infection, as at 11 dpf all larvae had succumbed to infection. Triplicate pools of 10 larvae were euthanized, washed and lysed as above. Total DNA was extracted with the Mobio PowerLyzer® Ultraclean® kit as described above and quantified with a NanoDrop® ND-1000 Spectrophotometer and sent to IMGM Laboratories GmbH (Germany) for Illumina sequencing. Primers Bakt_341F (5′-CCTACGGGNGGCWGCAG-3′ (SEQ ID NO:11)) and Bakt_805R (5′-GACTACHVGGGTATCTAATCC-3′ (SEQ ID NO:12)), amplifying variable regions 3 and 4 of the 16S gene were used for amplification [63].

Each amplicon was purified with solid phase reversible immobilization (SPRI) paramagnetic bead-based technology (AMPure XP beads, Beckman Coulter) with a Bead:DNA ratio of 0.7:1 (v/v) following manufacturer's instructions. Amplicons were normalized with the Sequal-Prep Kit (Life Technologies), so each sample contained approximately 1 ng/μl DNA. Samples, positive and negative controls were generated in one library. The High Sensitivity DNA LabChip Kit (was used on the 2100 Bioanalyzer system (both Agilent Technologies) to check the quality of the purified amplicon library. For cluster generation and sequencing, MiSeq® reagents kit 500 cycles Nano v2 (Illumina Inc.) was used. Before sequencing, cluster generation by two-dimensional bridge amplification was performed, followed by bidirectional sequencing, producing 2×250 bp paired-end (PE) reads.

MiSeq® Reporter 2.5.1.3 software was used for primary data analysis (signal processing, de-multiplexing, trimming of adapter sequences). CLC Genomics Workbench 8.5.1 (Qiagen) was used for read merging, quality trimming and QC reports and OTU definition were carried out in the CLC plugin Microbial Genomics module.

### Comparison of Whole Larvae Vs Intestinal Bacterial Content

Larvae reconventionalized with Mix10 and infected with F. columnare^{ALG} at 6 dpf for 3h were euthanized and washed. DNA was extracted from pools of 10 whole larvae or of pools of 10 intestinal tubes dissected with sterile surgical tweezer and subjected to Illumina 16S rDNA gene sequencing. GF larvae and dissected GF intestines were sampled as controls. No statistically significant difference was found between whole fish and gut bacterial content (p=0.99). Entire larvae were therefore used in the experiment monitoring bacterial establishment and recovery.

### Whole Genome Sequencing

Chromosomal DNA of the ten species composing the core of zebrafish larvae microbiota was extracted using the DNeasy Blood & Tissue kit (QIAGEN) including RNase treatment. DNA quality and quantity were assessed on a NanoDrop ND-1000 spectrophotometer (Thermo Scientific). DNA sequencing libraries were made using the Nextera DNA Library Preparation Kit (Illumina Inc.) and library quality was checked using the High Sensitivity DNA LabChip Kit on the Bioanalyzer 2100 (Agilent Technologies). Sequencing clusters were generated using the MiSeq reagents kit v2 500 cycles (Illumina Inc.) according to manufacturer's instructions. DNA was sequenced at the Helmholtz Centre for Infection Research by bidirectional sequencing, producing 2×250 bp paired-end (PE) reads. Between 1,108,578 and 2,914,480 reads per sample were retrieved with a median of 1,528,402. Reads were quality filtered, trimmed and adapters removed with fastq-mcf {Aronesty, 2011 #29} and genomes assembled using SPAdes 2.5.1 {Bankevich, 2012 #123}

Bacterial species identification. Whole genome-based bacterial species identification was performed by the TrueBac ID system (v1.92, DB:20190603) [https://www.truebacid.com/; [66]. Species-level identification was performed based on thealgorithmic cut-off set at 95% ANI when possible or when the 16S rDNA gene sequence similarity was >99%.

### Monitoring of Bacterial Dynamics

Three independent experiments were run over 6 weeks with eggs collected from different fish couples from different tanks to monitor establishment and recovery. Larvae were reared, sterilized and infected as above with the only difference that 75 cm³ culture flasks with vented caps (filled with 50 mL of sterile Volvic) were used to accommodate the larger number of larvae required, as in each experiment, larvae for time course Illumina sequencing were removed sequentially from the experiment that monitored the survival of the larvae. Animals were pooled (10 larvae for each time point/condition), euthanized, washed and lysed as described above and stored at −20° ° C. until the end of the survival monitoring, and until all triplicates had been collected.

### A) Community Establishment

In order to follow the establishment of the 10 core strains in the larvae, GF larvae were reconventionalized with an equiratio Mix10 as above. Reconv^{Mix10} larvae were sampled at 4 dpf immediately after addition of the 10 core species and then 20 min. 2h, 4h and 8h after. Germ-free, conventional larvae and the inoculum were also sampled as controls.

### B) Induction of Dysbiosis

Different doses of kanamycin (dose 1=200 μg/mL; dose 2=50 μg/ml; dose 3=25 μg/mL) and a penicillin/streptomycin antibiotic mix (dose 1=250 μg/mL; dose 2=15,6 μg/mL were tested on reconv^{Mix10} 4 dpf zebrafish larvae by adding them to the flask water to identify antibiotic treatments that were non-toxic to larvae but that caused dysbiosis.

After 16 hours of treatment, antibiotics were extensively washed off with sterile water and larvae were challenged with F. columnare^{ALG}, leading to the death of all larvae—e.g. successful abolition of colonization resistance with best results in all repeats with 250 μg/mL penicillin/streptomycin and 50 μg/mL kanamycin as antibiotic treatment.

### C) Community Recovery

As in B) after 8h of incubation, 4 dpf recon^{Mix10} larvae were treated with 250 μg/mL penicillin/streptomycin and 50 μg/mL kanamycin for 16h. Antibiotics were extensively washed off and larvae were now left to recover in sterile water for 24h to assess resilience of the bacterial community. Samples (pools of 10 larvae) were taken at 3h, 6h, 12h, 18h and 24h during recovery and sent for 16S rDNA Illumina sequencing. Larvae were then challenged at 6 dpf with F.columnare^{ALG} for 3h and survival was monitored daily for 10 days post-infection.

All time course samples were sequenced by IMGM Laboratories GmbH as described above.

### Statistical Analysis of Metataxonomic Data.

16S RNA analysis was performed with SHAMAN {Volant, 2019 #125}]. Library adapters, primer sequences, and base pairs occurring at 5′ and 3′ends with a Phred quality score <20 were trimmed off by using Alientrimmer (v0.4.0). Reads with a positive match against zebrafish genome (mm10) were removed. Filtered high-quality reads were merged into amplicons with Flash (v1.2.11). Resulting amplicons were clustered into operational taxonomic units (OTU) with VSEARCH (v2.3.4) [Rognes, T., Flouri, T., Nichols, B., Quince, C., & Mahé, F. (2016). VSEARCH: a versatile open source tool for metagenomics. PeerJ, 4, e2584.] The process includes several steps for de-replication, singletons removal, and chimera detection. The clustering was performed at 97% sequence identity threshold, producing 459 OTUs. The OTU taxonomic annotation was performed against the SILVA SSU (v132) database {Quast, 2012 #126} completed with 16S sequence of 10 bacterial communities using VSEARCH and filtered according to their identity with the reference {Yarza, 2014 #127}. Annotations were kept when the identity between the OTU sequence and reference sequence is ≥78.5% for taxonomic Classes, ≥82% for Orders, ≥86.5% for Families, ≥94.5% for Genera and ≥98% for species. Here, 73.2% of the OTUs set was annotated and 91.69% of them were annotated at genus level.

The input amplicons were then aligned against the OTU set to get an OTU contingency table containing the number of amplicon associated with each OTU using VSEARCH global alignment. The matrix of OTU count data was normalized for library size at the OTU level using a weighted non-null count normalization. Normalized counts were then summed within genera. The generalized linear model (GLM) implemented in the DESeq2 R package⁹⁵ was then applied to detect differences in abundance of genera between each group. We defined a GLM that included the treatment (condition) and the time (variable) as main effects and an interaction between the treatment and the time. Resulting P values were adjusted according to the Benjamini and Hochberg procedure.

The statistical analysis can be reproduced on shaman by loading the count table, the taxonomic results with the target and contrast files which are available on figshare https://doi.org/10.6084/m9.figshare. 11417082.v2.

### Determination of Cytokine Levels

Total RNAs from individual zebrafish larvae were extracted using RNeasy kit (Qiagen), 18h post pathogen exposure (12hs post-wash). Oligo(dT17)-primed reverse transcriptions were done using M-MLV H-reverse-transcriptase (Promega). Quantitative PCRs were performed using Takyon SYBR Green PCR Mastermix (Eurogentec) on a StepOne thermocycler (Applied Biosystems). Primers for ef1a (housekeeping gene, used for cDNA amount normalization), il1b, il10 and il22 are described in (Rendueles 2012). Data were analyzed using the ΔΔCt method. Four larvae were analyzed per condition. Zebrafish genes and proteins mentioned in the text: ef1a NM_131263; il1b BC098597; il22 NM_001020792; il10 NM_001020785; myd88 NM_212814

Histological Comparisons of GF, Conv and Re-Conv Fish GF and Conventional Fish Infected or not with F. columnare.

Fish were collected 24 h after infection (7 dpf) and were fixed for 24h at 4° C. in Trump fixative (4% methanol-free formaldehyde, 1% glutaraldehyde in 0.1 M PBS, PH 7.2) and sent to the PIBISA Microscopy facility (https://microscopies.med.univ-tours.fr/) in the Faculté de Médecine de Tours, (France) where whole fixed animals were processed, embedded in Epon. Semi-thin sections (1 μm) and cut using a X ultra-microtome and then either dyed with toluidine blue for observation by light microscopy and imaging or processed for Transmission electron microscopy.

Adult Zebrafish Pre-Treatment with C. massiliae

The zebrafish line AB was used. Fish were reared at 28° C. in dechlorinated recirculated water, then transferred in continuous flow aquaria when aging 3-4 months for infection experiments. C. massiliae was grown in TYES broth at 150 rpm and 28° C. until stationary phase. This bacterial culture was washed twice in sterile water and adjusted to OD₆₀₀ nm=1. Adult fish re-conventionalization was performed by adding C. massiliae bacterial suspension directly into the fish water (1L) at a final concentration of 2.10⁶ cfu/mL. Bacteria were maintained in contact with fish for 24 h by stopping the water flow then subsequently removed by restoring the water flow. C. massiliae administration was performed twice after water renewal. In the control group, the same volume of sterile water was added.

### Adult Zebrafish Infection Challenge

F. columnare infection was performed just after fish re-conventionalization with C. massiliae. The infection was performed as previously described by Li and co-workers with few modifications [Li et al., 2017]. Briefly, F. columnare strain ALG-0530 was grown in TYES broth at 150 rpm and 28° C. until late-exponential phase. Then, bacterial cultures were diluted directly into the water of aquaria (200 mL) at a final concentration of 5.10⁶ cfu/mL. Bacteria were maintained in contact with fish for 1 h by stopping the water flow then subsequently removed by restoring the water flow. Sterile TYES broth was used for the control group. Bacterial counts were determined at the beginning of the immersion challenge by plating serial dilutions of water samples on TYES agar. Water was maintained at 28° C. and under continuous oxygenation for the duration of the immersion. Groups were composed of 10 fish. Virulence was evaluated according to fish mortality 10 days post-infection.

Statistical methods. Statistical analyses were performed using unpaired, non-parametric Mann-Whitney test. Analyses were performed using Prism v8.2 (GraphPad Software). .

Evenness: The Shannon diversity index was calculated with the formula (Hs=−Σ[P(In(P)]) where P is the relative species abundance. Total evenness was calculated for the Shannon index as E=H_{S}/Hₘₐₓ. The less evenness in communities between the species (and the presence of a dominant species), the lower this index is.

### Handling of Rainbow Trout Larvae

The rainbow trout eggs post fertilization were obtained from Aqualande Group in France. Upon arrival, the eggs were acclimatized at 16° C. before their manipulation. All procedures were performed in a laminar microbiological cabinet and with single-use disposable plastic ware. Eggs were kept in 145×20 mm Petri dish until hatching in 75 mL autoclaved dechlorinated water. After hatching, fish were transferred and kept in 250 mL vented cap culture flasks in 100 mL sterile water at 16° C. Fish were fed 21 days post-hatching with irradiated powder food. To avoid waste accumulation and oxygen limitation, we renewed a half the volume of the water every two days to keep rainbow trout larvae healthy.

### Sterilization and Raising of Germ-Free Rainbow Trout

The rainbow trout eggs were first transferred to sterile Petri dish (140 mm. 150 eggs/dish) and washed twice with sterile methylene blue solution (0.05 mg/mL). Next, we kept freshly fertilized eggs in 75 mL of methylene blue solution and to be exposed to a cocktail of antibiotics previously described for 5 h (750 UL penicillin G (10,000U/mL)/streptomycin (10 mg/mL): 300 μL of filtered kanamycin sulfate (100 mg/mL) and 75 μL of antifungal drug Amphotericin B solution (250 μg/mL)) under agitation at room temperature. Then eggs were washed 3 times with fresh sterile water. After, they were bleached (0,005%) for 15 min. Eggs were washed again 3 times with sterile water. Afterwards, eggs were treated with Romeiod, an iodophore disinfection solution for 10 min. Finally, eggs were washed 3 times and we kept them at 16° C. in 75 mL of sterile water supplemented with antibiotics until hatching. Five to seven days after treatment eggs spontaneously hatched. Once hatched, fish were immediately transferred to 75 cm³ vented cap culture flasks containing 100 mL of fresh sterile water without antibiotics (12 larvae/flask). The hatching percentage was determined by counting hatched larvae in Petri dish to the total amount of eggs.

We monitored sterility at different moments during the experiment by spotting 50 μL of rearing water from each flask in LB agar plates, YPD agar and TYES agar, all incubated at 16° ° C. under aerobic conditions. We also checked fish larvae for bacterial contamination every week. Randomly chosen fish were sacrificed by an overdose of filtered tricaine methane sulfonate solution (MS222, 300 mg/L). Whole fish were mechanically disrupted in Lysing Matrix tubes containing 1 mL of sterile water and 425-600 μm glass beads (Sigma). Samples were homogenized at 6.0 m s⁻¹ for 45 s on a FastPrep-24 instrument (XXX). Serial dilutions of the homogenized solution were plated on TYES agar, YPD agar and LB agar. When water samples or collected euthanized and homogenized fish showed any bacterial CFU over any of different culture media used, these animals (or flask) were removed from the experiment. The absence of any contamination in the fish larvae was further confirmed by PCR using primers specific for the chromosomal 16S region (27F: 5′-AGAGTTTGATCCTGGCTCAG-3′ (SEQ ID NO: 13); 1492R 5′-GGTTACCTTGTTACGACTT-3′ (SEQ ID NO:14)) [177].

### Bacterial Strains and Growth Conditions

Bacterial strains used in this study are listed in Table 2. F. columnare strains Fc7 and IA-S-4, and Chryseobacterium massiliae were grown at 150 rpm and 18° ° C. in tryptone yeast extract salts (TYES) broth [0.4% (w/v) tryptone, 0.04% yeast extract. 0.05% (w/V) MgSO₄ 7H₂O, 0.02% (w/v), CaCl₂) 2H₂O, 0.05% (w/v) D-glucose, pH 7.2]. F. psychrophilum strains THCO2-90 and FRGDSA 1882/11 were grown in TYES broth at 150 rpm and 28° C. Yersinia ruckeri strain JIP 27/88 was grown in Luria-Bertani (LB) medium at 150 rpm and 28° C. V. anguillarum strain 1669 was grown in tryptic soy broth (TSB) at 150 rpm and 28° C. L. garvieae was grown in brain heart infusion (BHI) broth at 150 rpm and 28° C. If required, 15 g/L of agar was added for solid medium. Stock cultures were preserved at −80° ° C. in respective broth containing 20% (vol/vol) glycerol.

### Fish Infection Challenge

Pathogenic bacteria were grown in suitable media at different temperatures until advanced stationary phase. Then, each culture was pelleted (10,000 rpm for 5 min) and washed once in sterile water. Bacteria were resuspended and bacteria were added to culture flasks at a final concentration 10⁷ cfu/mL. After 24 hours of incubation with pathogenic bacteria at 16° C., fish were washed three times by water renewing. Between 10 to 12 larvae were used per condition per experiment. Bacterial counts were determined at the beginning and at the end of immersion challenge by plating serial dilutions of water samples on specific medium for each pathogen. Each experiment was repeated at least 2 times. Virulence was evaluated according to fish mortality 10 days post-infection.

### Characterization of Culturable Conventional Rainbow Trout Microbiota

To identify the species constituting the cultivable conventional microbiota, 3 conventional rainbow trout larvae were sacrificed with an overdose of MS222 at 31 dph. These fish were homogenized following the protocol described above and serial dilutions of homogenized suspension were plated on different culture media: TYES agar, LB agar, R2A agar and TSA. The plates were incubated a 16° C. for 48 to 72 hours. After incubation, each morphologically distinct colony (based on form, size, color, texture, elevation and margin) were isolated and conserved at −80° C. in respective broth containing 15% (vol/vol) glycerol. Individual 16S-based identification by amplifying and sequencing the 16S chromosomal region using the universal oligonucleotides 27F and 1492R. Afterwards, 16S rRNA gene sequences were compared with those available in the EzBioCloud database [178].

### Germ Free Rainbow Trout Microbial Re-Conventionalization

Each isolated bacterial species was grown for 24 h in suitable media at 150 rpm and 28° C. Bacteria were then pelleted and washed twice in sterile water. They were diluted at a final concentration of 5×10⁷ cfu/mL. At 22 dph, germ-free rainbow trout were re-conventionalized by adding 1 mL of each bacterial suspension into the flasks (5×10⁵ cfu/mL, final concentration). In case of fish re-conventionalization with bacteria consortia, after bacterial washes, all isolated species were mixed in an aqueous suspension, each at a concentration of 5×10⁷ cfu/mL. Afterward, this mixed bacterial suspension was added to the flask containing germ-free rainbow trout as described. In all cases, fish re-conventionalization was performed for 48 h, followed by the infection challenge with F. columnare. Bacterial suspensions were added immediately after water renewing. Each experiment was repeated at least 2 times.

### Histological Examination

Histological sections were used to compare microscopical lesions between GF and conventional fish after infection with F. columnare. Sacrificed animals were fixed for 24h at 4° C. in Trump fixative (4% methanol-free formaldehyde, 1% glutaraldehyde in 0.1 M PBS, pH 7.2) [179]. Whole fixed animals were processed and then blocked in Epon. Semi-thin sections (1 μm) were cut using an ultra-microtome and stained with toluidine blue for observation by light microscopy and imaging.

3D Imaging of Cleared Fish by Optical Projection Tomography (iDISCO)

For a 3D imaging of cleared whole fish, Fishes were fixed with 4% formaldehyde in PBS overnight at 4° C. Fixed samples were rinsed with PBS. To render tissue transparent, fishes were first depigmented by pretreatment in SSC 0.5× twice during 1h at RT followed by an incubation in SSC 0.5×+KOH 0.5%+H₂O₂ 3% during 2h at RT. Depigmentation was stopped by incubation in PBS twice during 15 min. Then fishes were post-fixed with 2% formaldehyde in PBS during 2h at RT and then rinsed twice with PBS for 30 min. Depigmented fishes were cleared with the iDISCO+ protocol [Renier, 2016] (Renier et al 2016,PMID 27238021). Briefly, samples were progressively dehydrated in ascending methanol series (20%, 40%, 60%, 80% in H2O and 100% twice) during 1 hour for each step. The dehydrated samples were bleached by incubation in methanol+5% H₂O₂ at 4° C. overnight, followed by incubation in methanol 100% twice for 1h. They were then successively incubated in 67% dichloromethane+33% methanol during 3 hours, dichloromethane during 1 hour and finally dibenzylether until fishes became completely transparent. Whole sample acquisition was performed on a light-sheet ultramicroscope (LaVision Biotec, Bielefeld, Germany) with a 2× objective using a 0.63× zoom factor. Autofluorescence was acquired by illuminating both sides of the sample with 488 nm laser. Z-stacks were acquired with a 2 μm z-step.

### Whole Genome Sequencing.

Chromosomal DNA of Flavobacterium sp. strain 4466 isolated from rainbow trout larvae microbiota was extracted using the DNeasy Blood & Tissue kit (QIAGEN) including RNase treatment. DNA quality and quantity was assessed on a NanoDrop ND-1000 spectrophotometer (Thermo Scientific). DNA sequencing libraries were made using the Nextera DNA Library Preparation Kit (Illumina inc.) and library quality was checked using the High Sensitivity DNA LabChip Kit on the Bioanalyzer 2100 (Agilent Technologies). Sequencing clusters were generated using the MiSeq reagents kit v2 500 cycles (Illumina Inc.) according to manufacturer's instructions. DNA was sequenced at the Mutualized Platform for Microbiology at Institut Pasteur by bidirectional sequencing, producing 2×150 bp paired-end (PE) reads. Reads were quality filtered, trimmed and adapters removed with fastq-mcf and genomes assembled using SPAdes 3.9.0 [219].

### Phylogenomic Analysis.

The proteomes for the 15 closest Flavobacterium strains identified by the ANI analysis were retrieved from the NCBI RefSeq database (Table below).

| Specie | Assembly | Host | BioSample | FTP |
|---|---|---|---|---|
| F. tructae | GCF_002217475.1 | Oncorhynchus mykiss | SAMN06049067 | https://ftp.ncbi.nlm.nih.gov/genomes/all/ |
| | | | | GCF/002/217/475/GCF_002217475.1_ASM221747v1/ |
| F. spartasanii | GCF_002217445.1 | Oncorhynchus | SAMN06049056 | https://ftp.ncbi.nlm.nih.gov/genomes/all/ |
| | | tshawytscha | | GCF/002/217/445/GCF_002217445.1_ASM221744v1/ |
| F. chilense | GCF_001602525.1 | Environment | SAMN04506025 | https://ftp.ncbi.nlm.nih.gov/genomes/all/ |
| | | (Loyalsock Creek, | | GCF/001/602/525/GGF_001602525.1_ASM160252v1/ |
| | | USA) | | |
| F. plurextorum | GCF_002217395.1 | Oncorhynchus mykiss | SAMN06049068 | https://ftp.ncbi.nlm.nih.gov/genomes/all/ |
| | | | | GCF/002/217/395/GCF_002217395.1_ASM221739v1/ |
| F. oncorhynchi | GCF_002217355.1 | Oncorhynchus mykiss | SAMN06049060 | https://ftp.ncbi.nlm.nih.gov/genomes/all/ |
| | | | | GCF/002/217/355/GCF_002217355.1_ASM221735v1/ |
| F. denitrificans | GCF_000425445.1 | Aporrectodea | SAMN02441540 | https://ftp.ncbi.nlm.nih.gov/genomes/all/ |
| | | caliginosa | | GCF/000/425/445/GCF_000425445.1_ASM42544v1/ |
| F. cutihirudines | GCF_003385895.1 | Hirudo verbana | SAMN05444268 | https://ftp.ncbi.nlm.nih.gov/genomes/all/ |
| | | | | GCF/003/385/895/GCF_003385895.1_ASM338589v1/ |
| F. aurantiacus | GCF_000016645.1 | NA | SAMN02598357 | https://ftp.ncbi.nlm.nih.gov/genomes/all/ |
| | | | | GCF/000/016/645/GCF_000016645.1_ASM1664v1/ |
| F. hibernum | GCF_000832125.1 | Environment | SAMN02934118 | https://ftp.ncbi.nlm.nih.gov/genomes/all/ |
| | | (freshwater Antarctic | | GCF/000/832/125/GCF_000832125.1_ASM83212v1/ |
| | | lake) | | |
| F. piscis | GCF_001686925.1 | NA | SAMN04570197 | https://ftp.ncbi.nlm.nih.gov/genomes/all/ |
| | | | | GCF/001/686/925/GCF_001686925.1_ASM168692v1/ |
| F. frigidimoris | GCA_900129595.1 | Environmental | SAMN05444481 | https://ftp.ncbi.nlm.nih.gov/genomes/all/ |
| | | (Antarctic seawater) | | GCA/900/129/595/GCA_900129595.1_IMG- |
| | | | | taxon_2695420960_annotated_assembly/ |
| F. araucananum | GCF_002222055.1 | Salmo salar | SAMN06049049 | https://ftp.ncbi.nlm.nih.gov/genomes/all/ |
| | | | | GCF/002/222/055/GCF_002222055.1_ASM222205v1/ |
| F. sp. Leaf82 | GCF_001422725.1 | Arabidopsis thaliana | SAMN04151618 | https://ftp.ncbi.nlm.nih.gov/genomes/all/ |
| | | | | GCF/001/422/725/GCF_001422725.1_Leaf82/ |
| F. sp. LM4 | GCF_002017935.1 | Environmental (Lake | SAMN06263772 | https://ftp.ncbi.nlm.nih.gov/genomes/all/ |
| | | Michigan, USA) | | GCF/002/017/935/GCF_002017935.1_ASM201793v1/ |
| F. pectinovorum | GCF_900142715.1 | NA | SAMN0S444387 | https://ftp.ncbi.nlm.nih.gov/genomes/all/ |
| | | | | GCF/900/142/715/GCF_900142715.1_IMG- |
| | | | | taxon_2698536748_annotated_assembly/ |
| F. sp. GV028 | GCF_003386855.1 | NA | SAMN08778959 | https://ftp.ncbi.nlm.nih.gov/genomes/all/ |
| | | | | GCF/003/386/855/GCF_003386855.1_ASM338685v1/ |

These sequences together with the Flavobacterium sp. strain UGB 4466 proteome were analyzed with Phylophlan (version 0.43, march 2020) [220]. This method uses the 400 most conserved proteins across the proteins and builds a Maximum likelihood phylogenetic tree using RAxML (version 8.2.8) [221]. Maximum likelihood tree was boostrapped with 1000 replicates.

### Agar Overlay Assay for Growth Inhibition Detection

The growth inhibitory effect of Flavobacterium sp. 4466 has been evaluated using an agar spot test. Briefly. 125 μl from an overnight culture of different strains of F. columnare adjusted to OD 1 were mixed to 5 ml of top agar (0.7% agar) and overlaid on plates of TYES agar. Five μL of overnight culture of Flavobacterium sp. 4466 were then spotted on the overlay of targeted bacteria. The plates were incubated at 28° ° C. for 24 hours. Growth inhibition of F. columnare was recorded by observation of a clear halo surrounding Flavobacterium sp. colony. Sterile TYES broth was used as a mock and the experiment were performed in triplicate.

### Whole Genome Sequencing for Taxonomic Identification, Antimicrobial Resistance and Virulence Factor Prediction Based on Whole Genome Sequence Analysis

Chromosomal DNA of Chryseobacterium sp, Delftia sp. strain 4465 (available in ENA (European Nucleotide Archive) database under primary accession number ERS4574863 (version 1) and secondary accession number SAMEA6847265 (Tax ID 80866, scientific name Delftia acidovorans), and Flavobacterium sp. strain 4466 was extracted using the DNeasy Blood & Tissue kit (QIAGEN) including RNase treatment. DNA quality and quantity were assessed on a NanoDrop ND-1000 spectrophotometer (Thermo Scientific). DNA sequencing libraries were made using the Nextera DNA Library Preparation Kit (Illumina Inc.) and library quality was checked using the High Sensitivity DNA LabChip Kit on the Bioanalyzer 2100 (Agilent Technologies). Sequencing clusters were generated using the MiSeq reagent kit with v2 chemistry for 500 cycles (Illumina Inc.) according to manufacturer's instructions. DNA was sequenced at the Mutualized Platform for Microbiology at Institut Pasteur with bidirectional sequencing, producing 2×150 bp paired-end (PE) reads. Reads were quality filtered, trimmed and adapters removed with fastq-mcf and genomes assembled using SPAdes 3.9.0 [202].

### Whole Genome Analysis for Taxonomic Identification, Antimicrobial Resistance and Virulence Factor Prediction Based on Whole Genome Sequence Analysis

A whole genome analysis was performed for Chryseobacterium sp., Delftia sp. strain 4465 (available in ENA (European Nucleotide Archive) database under primary accession number ERS4574863 (version 1) and secondary accession number SAMEA6847265 (Tax ID 80866, scientific name Delftia acidovorans)), and Flavobacterium sp. strain 4466 with the TrueBac ID system (v1.92, DB:20190603) (https://www.truebacid.com/) [203]. Species-level identification was performed based on the algorithmic cut-off set at 95% Average Nucleotide Identity (ANI), or when the 16S rRNA gene sequence similarity was >99%. Virulence factors were identified using Virulence Factors Database (VFDB, http://www.mgc.ac.cn/VFs/). Antimicrobial resistance (AMR) gene(s) were found using AMRFinderPlus, a tool that identifies AMR genes using either protein annotations or nucleotide sequence via National Center for Biotechnology Information (https://www.ncbi.nlm.nih.gov/pathogens/antimicrobialresistance/AMRFinder/).

### REFERENCES

1. Rolig, A. S., et al., Individual Members of the Microbiota Disproportionately Modulate Host Innate Immune Responses. Cell Host Microbe, 2015. 18(5): p. 613-20.
2. McFall-Ngai, M. J., Unseen forces: the influence of bacteria on animal development. Dev Biol, 2002. 242(1): p. 1-14.
3. Cani, P. D. and N. M. Delzenne, The role of the gut microbiota in energy metabolism and metabolic disease. Curr Pharm Des, 2009. 15(13): p. 1546-58.
4. Falcinelli, S., et al., Lactobacillus rhamnosus lowers zebrafish lipid content by changing gut microbiota and host transcription of genes involved in lipid metabolism. Sci Rep, 2015. 5: p. 9336.
5. Stecher, B. and W. D. Hardt, The role of microbiota in infectious disease. Trends Microbiol, 2008. 16(3): p. 107-14.
6. Stecher, B. and W. D. Hardt, Mechanisms controlling pathogen colonization of the gut. Curr Opin Microbiol, 2011. 14(1): p. 82-91.
7. van der Waaij. D., J. M. Berghuis-de Vries, and J. E. C. Lekkerkerk-van der Wees, Colonization resistance of the digestive tract and the spread of bacteria to the lymphatic organs in mice. The Journal of Hygiene, 1972. 70: p. 335-342.
8. Littman, D. R. and E. G. Pamer, Role of the commensal microbiota in normal and pathogenic host immune responses. Cell Host Microbe, 2011. 10(4): p. 311-23.
9. Heselmans, M., et al., Gut flora in health and disease: potential role of probiotics. Curr Issues intest Microbiol, 2005. 6(1): p. 1-7.
10. Boirivant, M. and W. Strober, The mechanism of action of probiotics. Curr Opin Gastroenterol, 2007. 23(6): p. 679-92.
11. Robinson, C. J., B. J. Bohannan, and V. B. Young, From structure to function: the ecology of host-associated microbial communities. Microbiol Mol Biol Rev. 2010. 74(3): p. 453-76.
12. Vollaard, E. J. and H. A. Clasener, Colonization resistance. Antimicrob Agents Chemother, 1994. 38(3): p. 409-14.
13. Gill, H. S., Probiotics to enhance anti-infective defences in the gastrointestinal tract. Best Pract Res Clin Gastroenterol, 2003. 17(5): p. 755-73.
14. Falcinelli, S., et al., Probiotic treatment reduces appetite and glucose level in the zebrafish model. Sci Rep, 2016. 6: p. 18061.
15. Rawls, J. F., B. S. Samuel, and J. I. Gordon, Gnotobiotic zebrafish reveal evolutionarily conserved responses to the gut microbiota. Proc Natl Acad Sci USA, 2004. 101(13): p. 4596-601.
16. Meeker, N. D. and N. S. Trede, Immunology and zebrafish: spawning new models of human disease. Dev Comp Immunol, 2008. 32(7): p. 745-57.
17. Kanther, M. and J. F. Rawls, Host-microbe interactions in the developing zebrafish. Curr Opin Immunol, 2010. 22(1): p. 10-9.
18. Milligan-Myhre, K., et al., Study of host-microbe interactions in zebrafish. Methods Cell Biol, 2011. 105: p. 87-116.
19. Burns, A. R. and K. Guillemin, The scales of the zebrafish: host-microbiota interactions from proteins to populations. Curr Opin Microbiol, 2017. 38: p. 137-141.
20. Douglas, A. E., Simple animal models for microbiome research. Nat Rev Microbiol, 2019.
21. Melancon, E., et al., Best practices for germ-free derivation and gnotobiotic zebrafish husbandry. Methods Cell Biol, 2017. 138: p. 61-100.
22. Cantas, L., et al., Culturable gut microbiota diversity in zebrafish. Zebrafish, 2012. 9(1): p. 26-37.
23. Rendueles, O., et al., A new zebrafish model of oro-intestinal pathogen colonization reveals a key role for adhesion in protection by probiotic bacteria. PLoS Pathog. 2012. 8(7): p. e 1002815.
24. Caruffo, M., et al., Protective Yeasts Control V. anguillarum Pathogenicity and Modulate the Innate Immune Response of Challenged Zebrafish (Danio rerio) Larvae. Front Cell Infect Microbiol, 2016. 6: p. 127.
25. Perez-Ramos, A., et al., beta-Glucan-Producing Pediococcus parvuius 2.6: Test of Probiotic and Immunomodulatory Properties in Zebrafish Models. Front Microbiol, 2018. 9: p. 1684.
26. Girija, V., et al., In vitro antagonistic activity and the protective effect of probiotic Bacillus licheniformis Dahb1 in zebrafish challenged with GFP tagged Vibrio parahaemolyticus Dahv2. Microb Pathog, 2018. 114: p. 274-280.
27. Lin. Y. S., et al., Dietary administration of Bacillus amyloliquefaciens R8 reduces hepatic oxidative stress and enhances nutrient metabolism and immunity against Aeromonas hydrophila and Streptococcus agalactiae in zebrafish (Danio rerio). Fish Shellfish Immunol, 2019. 66: p. 410-419.
28. Qin, C., et al., EPSP of L. casei BL.23 Protected against the Infection Caused by Aeromonas veronii via Enhancement of Immune Response in Zebrafish. Front Microbiol, 2017. 8: p. 2406.
29. Wang, Y., et al., Two highly adhesive lactic acid bacteria strains are protective in zebrafish infected with Aeromonas hydrophila by evocation of gut mucosal immunity. J Appl Microbiol, 2016. 120(2): p. 441-51.
30. Chu, W., et al., Quorum quenching bacteria Bacillus sp. QSI-1 protect zebrafish (Danio rerio) from Aeromonas hydrophila infection. Sci Rep, 2014. 4: p. 5446.
31. Declercq, A. M., et al., Columnaris disease in fish: a review with emphasis on bacterium-host interactions. Vet Res. 2013. 44: p. 27.
32. Soto, E., et al., Genetic and virulence characterization of Flavobacterium columnare from channel catfish (Ictalurus punctatus). J Appl Microbiol, 2008. 104(5): p. 1302-10.
33. Li. N., et al., The Type IX Secretion System is Required for Virulence of the Fish Pathogen Flavobacterium columnare. Appl Environ Microbiol, 2017. 83(23).
34. Olivares-Fuster, O., et al., Adhesion dynamics of Flavobacterium columnare to channel catfish Ictalurus punctatus and zebrafish Danio rerio after immersion challenge. Dis Aquat Organ, 2011. 96(3): p. 221-7.
35. van der Vaart, M., et al., Functional analysis of a zebrafish myd88 mutant identifies key transcriptional components of the innate immune system. Dis Model Mech, 2013. 6(3): p. 841-54.
36. Stephens, W. Z., et al., The composition of the zebrafish intestinal microbial community varies across development. ISME J. 2016. 10(3): p. 644-54.
37. Jacobson, A., et al., A Gut Commensal-Produced Metabolite Mediates Colonization Resistance to Salmonella Infection. Cell Host Microbe, 2018. 24(2): p. 296-307.e7.
38. Chatzidaki-Livanis, M., M. J. Coyne, and L. E. Comstock, An antimicrobial protein of the gut symbiont Bacteroides fragilis with a MACPF domain of host immune proteins. Mol Microbiol, 2014. 94(6): p. 1361-74.
39. Roelofs, K. G., et al., Bacteroidales Secreted Antimicrobial Proteins Target Surface Molecules Necessary for Gut Colonization and Mediate Competition In Vivo. MBio, 2016. 7(4).
40. Russell, A. B., et al., A type VI secretion-related pathway in Bacteroidetes mediates interbacterial antagonism. Cell Host Microbe, 2014. 16(2): p. 227-236.
41. Kamada, N., et al., Role of the gut microbiota in immunity and inflammatory disease. Nat Rev Immunol, 2013. 13(5): p. 321-35.
42. Ganz, J., E. Melancon, and J. S. Eisen, Zebrafish as a model for understanding enteric nervous system interactions in the developing intestinal tract. Methods Cell Biol, 2016. 134: p. 139-64.
43. Bates, J. M., et al., Distinct signals from the microbiota promote different aspects of zebrafish gut differentiation. Dev Biol, 2006 297(2): p. 374-86.
44. Yan, Q., C. J. van der Gast, and Y. Yu, Bacterial community assembly and turnover within the intestines of developing zebrafish. PLOS One, 2012. 7(1): p. e30603.
45. Costello. E. K., et al., The application of ecological theory toward an understanding of the human microbiome. Science, 2012. 336(6086): p. 1255-62
46. Rogers, G. B., et al., Interpreting infective microbiota: the importance of an ecological perspective. Trends Microbiol, 2013. 21(6): p. 271-6.
47. Burns, A. R., et al., Contribution of neutral processes to the assembly of gut microbial communities in the zebrafish over host development. ISME J. 2016. 10(3): p. 655-64.
48. Hibbing, M. E., et al., Bacterial competition: surviving and thriving in the microbial jungle Nat Rev Microbiol, 2010. 8(1): p. 15-25.
49. Shafquat, A., et al., Functional and phylogenetic assembly of microbial communities in the human microbiome. Trends Microbiol, 2014. 22(5): p. 261-6.
50. Shade. A., et al., Fundamentals of microbial community resistance and resilience. Front Microbiol, 2012. 3: p. 417.
51. Willing, B. P., S. L. Russell, and B. B. Finlay, Shifting the balance: antibiotic effects on host—microbiota mutualism. Nat Rev Microbiol, 2011. 9(4): p. 233-43.
52. Brugman, S., et al., Oxazolone-induced enterocolitis in zebrafish depends on the composition of the intestinal microbiota. Gastroenterology, 2009. 137(5): p. 1757-67 e1.
53. Salonen, A., et al., Comparative analysis of fecal DNA extraction methods with phylogenetic microarray: effective recovery of bacterial and archaeal DNA using mechanical cell lysis. J Microbiol Methods, 2010. 81(2): p. 127-34.
54. Kunin. V., et al., Wrinkles in the rare biosphere: pyrosequencing errors can lead to artificial inflation of diversity estimates. Environ Microbiol, 2010. 12(1): p. 118-23.
55. Darwish, A. M. and A. A. Ismaiel, Genetic diversity of Flavobacterium columnare examined by restriction fragment length polymorphism and sequencing of the 16S ribosomal RNA gene and the 16S-23S rDNA spacer. Mol Cell Probes, 2005. 19(4): p. 267-74.
56. LaFrentz. B. R., et al., Identification of Four Distinct Phylogenetic Groups in Flavobacterium columnare With Fish Host Associations. Front Microbiol, 2018. 9: p. 452.
57. Bernardet, J.-F. and J. P. Bowman, The Genus Flavobacterium. Prokaryotes, 2006. 7: p. 481-531.
58. Kunttu, H. M., et al., Virulent and nonvirulent Flavobacterium columnare colony morphologies: characterization of chondroitin AC lyase activity and adhesion to polystyrene. J Appl Microbiol, 2011. 111(6): p. 1319-26
59. Suomalainen, L. R., M. Tlirola, and E. T. Valtonen, Chondroitin AC lyase activity is related to virulence of fish pathogenic Flavobacterium columnare. J Fish Dis, 2006. 29(12): p. 757-63.
60. Olivares-Fuster, O. and C. R. Arias, Use of suppressive subtractive hybridization to identify Fiavobacterium columnare DNA sequences not shared with Flavobacterium johnsoniae. Lett Appl Microbiol, 2008. 46(6): p. 605-12.
61. Shabgah, A. G., et al., Interleukin-22 in human inflammatory diseases and viral infections. Autoimmun Rev, 2017. 16(12): p. 1209-1218.
62. Verschuere, L., et al., Probiotic bacteria as biological control agents in aquaculture. Microbiol Moi Biol Rev, 2000. 64(4): p. 655-71.
63. Lemon, K. P., et al., Microbiota-targeted therapies: an ecological perspective. Sci Transl Med, 2012. 4(137): p. 137rv5.
64. Garcia, J. C., et al., Characterization of atypical Flavobacterium columnare and identification of a new genomovar. J Fish Dis, 2018. 41(7): p. 1159-1164.
65. Pham, L. N., et al., Methods for generating and colonizing gnotobiotic zebrafish. Nat Protoc, 2008. 3(12): p. 1862-75.
66. Ha, S. M., et al., Application of the Whole Genomne-Based Bacterial Identification System, TrueBac ID, Using Clinical Isolates That Were Not Identified With Three Matrix-Assisted Laser Desorption/Ionization Time-of-Flight Mass Spectrometry (MALDI-TOF MS) Systems. Ann Lab Med. 2019. 39(6): p. 530-536.
67. McDowell, E. M. and B. F. Trump. Histologic fixatives suitable for diagnostic light and electron microscopy. Arch Pathol Lab Med, 1976. 100(8): p. 405-14.
101. FAO, The state of World Fisheries and Aquaculture, F.a.A. Organization, Editor. 2016, Food and Agriculture Organization of the United Nations: Rome.
102. Ashley, P. J., Fish welfare: Current issues in aquaculture. Applied Animal Behaviour Science, 2007. 104(3): p. 199-235.
103. Vadstein, O., et al., Microbiology and immunology of fish larvae. Reviews in Aquaculture, 2013. 5(s1): p. S1-S25.
104 Kennedy, D. A., et al., Potential drivers of virulence evolution in aquaculture. Evolutionary applications, 2016. 9(2): p. 344-354.
105. Embregts, C. W. and M. Forlenza, Oral vaccination of fish: Lessons from humans and veterinary species. Dev Comp Immunol, 2016. 64: p. 118-37.
106. Perez-Sanchez, T., B. Mora-Sanchez, and J. L. Balcazar, Biological Approaches for Disease Control in Aquaculture: Advantages, Limitations and Challenges. Trends Microbiol, 2018. 26(11): p. 896-903.
107. Cabello, F. C., et al., Antimicrobial use in aquaculture re-examined: its relevance to antimicrobial resistance and to animal and human health. Environmental Microbiology, 2013. 15(7): p. 1917-1942.
108. Santos, L. and F. Ramos, Analytical strategies for the detection and quantification of antibiotic residues in aquaculture fishes: A review. Trends in Food Science & Technology, 2016. 52: p. 16-30.
109. Santos, L. and F. Ramos, Antimicrobial resistance in aquaculture: Current knowledge and alternatives to tackle the problem. International Journal of Antimicrobial Agents, 2018. 52(2): p. 135-143.
110. Brandt, K. K., et al., Ecotoxicological assessment of antibiotics: A call for improved consideration of microorganisms. Environ Int, 2015. 85: p. 169-205.
111. Defoirdt. T., P. Sorgeloos, and P. Bossier, Alternatives to antibiotics for the control of bacterial disease in aquaculture. Curr Opin Microbiol, 2011. 14(3): p. 251-8.
112. Tamnecki, A. M., et al., Benefits of a Bacillus probiotic to larval fish survival and transport stress resistance. Scientific Reports, 2019. 9(1): p. 4892.
113. Verschuere, L., et al., Probiotic Bacteria as Biological Control Agents in Aquaculture. Microbiology and Molecular Biology Reviews, 2000. 64(4): p. 655-671.
114. FAO and WHO, Report of a joint FAO/WHO expert consultation on evaluation of health and nutritional properties of probiotics in food including powder milk with live lactic acid bacteria. 2001. Food and Agriculture Organization of the United Nations, World Health Organization: Cordoba, Argentina.
115. Tinh, N. T., et al., A review of the functionality of probiotics in the larviculture food chain. Mar Biotechnol (NY), 2008. 10(1): p. 1-12.
116. Martinez Cruz. P., et al., Use of probiotics in aquaculture. ISRN microbiology, 2012. 2012: p. 916845-916845.
117. Buffie, C. G. and E. G. Parner, Microbiota-mediated colonization resistance against intestinal pathogens. Nature Reviews Immunology, 2013. 13: p. 790.
118. Stecher, B. and W.-D. Hardt, Mechanisms controlling pathogen colonization of the gut. Current Opinion in Microbiology, 2011. 14(1): p. 82-91.
119. Olsan, E. E., et al., Colonization resistance: The deconvolution of a complex trait. J Biol Chem, 2017. 292(21): p. 8577-8581.
120. de Bruijn, I., et al., Exploring fish microbial communities to mitigate emerging diseases in aquaculture. FEMS Microbiology Ecology, 2017. 94(1)
121. Gomez-Gil, B., A. Roque, and J. F. Turnbull, The use and selection of probiotic bacteria for use in the culture of larvai aquatic organisms. Aquaculture, 2000. 191(1): p. 259-270.
122. Xiong, J. B., L. Nie, and J. Chen, Current understanding on the roles of gut microbiota in fish disease and immunity. Zool Res, 2019. 40(2): p. 70-76.
123. Hai, N. V., The use of probiotics in aquaculture. Journal of Applied Microbiology, 2015. 119(4): p. 917-935.
124. Lazado. C. C. and C. M. A. Caipang. Mucosal immunity and probiotics in fish. Fish & Shellfish Immunology, 2014. 39(1): p. 78-89.
125. Dierckens, K., et al., Development of a bacterial challenge test for gnotobiotic sea bass (Dicentrarchus labrax) larvae. Environmental Microbiology, 2009. 11(2): p. 526-533.
126. Fiebiger, U., S. Bereswill, and M. M. Heimesaat, Dissecting the Interplay Between intestinal Microbiota and Host immunity in Health and Disease: Lessons Learned from Germfree and Gnotobiotic Animal Models. Eur J Microbiol Immunol (Bp), 2016. 6(4): p. 253-271.
127. Yi, P. and L. Li, The germfree murine animal: An important animal model for research on the relationship between gut microbiota and the host. Veterinary Microbiology, 2012. 157(1): p. 1-7.
128. Forberg, T., A. Arukwe, and O. Vadstein, A protocol and cultivation system for gnotobiotic Atlantic cod larvae (Gadus morhua L.) as a tool to study host microbe interactions. Aquaculture, 2011. 315(3): p. 222-227.
129. Verner-Jeffreys, D. W., R. J. Shields, and T. H. Birkbeck, Bacterial influences on Atlantic halibut Hippoglossus hippoglossus yolk-sac larval survival and start-feed response. Dis Aquat Organ, 2003. 56(2): p. 105-13.
130. Munro, P. D., A. Barbour, and T. H. Birkbeck, Comparison of the Growth and Survival of Larval Turbot in the Absence of Culturable Bacteria with Those in the Presence of Vibrio anguillarum, Vibrio alginolyticus, or a Marine Aeromonas sp. Applied and Environmental Microbiology, 1995. 61(12): p. 4425-4428.
131. Forberg, T. and K. Milligan-Myhre, Chapter 6—Gnotobiotic Fish as Models to Study Host-Microbe Interactions, in Gnotobiotics, T. R. Schoeb and K. A. Eaton, Editors. 2017, Academic Press. p. 369-383.
132. Vestrum R. I., et al., Emerging Issues in Fish Larvae Research, Y. M., Editor. 2018, Springer.
133. Caruffo, M., et al., Protective Yeasts Control V. anguillarum Pathogenicity and Modulate the Innate Immune Response of Challenged Zebrafish (Danio rerio) Larvae. Front Cell Infect Microbiol, 2016. 6: p. 127.
134. Perez-Ramos, A., et al., beta-Glucan-Producing Pediococcus parvuius 2.6: Test of Probiotic and Immunomodulatory Properties in Zebrafish Models. Front Microbiol, 2018. 9: p. 1684.
135. Qin. C., et al., EPSP of L. casei BL23 Protected against the Infection Caused by Aeromonas veronii via Enhancement of Immune Response in Zebrafish. Frontiers in Microbiology, 2017. 8(2406).
136. Rendueles, O., et al., A new zebrafish model of oro-intestinal pathogen colonization reveals a key role for adhesion in protection by probiotic bacteria. PLOS Pathog. 2012. 8(7): p. e 1002815.
137. Declercq. A. M., et al., Columnaris disease in fish: a review with emphasis on bacterium-host interactions. Veterinary Research, 2013. 44(1): p. 27.
138. Renier, N., et al., IDISCO: A Simple, Rapid Method to Immunoiabel Large Tissue Samples for Volume Imaging. Cell, 2014. 159(4): p. 896-910.
139. Butt. R. L. and H. Volkoff, Gut Microbiota and Energy Homeostasis in Fish. Frontiers in endocrinology, 2019. 10: p. 9-9.
140. Banerjee, G. and A. K. Ray, The advancement of probiotics research and its application in fish farming industries. Research in Veterinary Science, 2017. 115: p. 66-77.
141. Hoseinifar, S. H., et al., Probiotics as Means of Diseases Control in Aquaculture, a Review of Current Knowledge and Future Perspectives. Frontiers in Microbiology, 2018. 9(2429).
142. Rawls, J. F., B. S. Samuel, and J. I. Gordon, Gnotobiotic zebrafish reveal evolutionarily conserved responses to the gut microbiota. Proceedings of the National Academy of Sciences of the United States of America, 2004. 101(13): p. 4596-4601.
143. Milligan-Myhre, K., et al., Innate immune responses to gut microbiota differ between oceanic and freshwater threespine stickleback populations. Disease Models &amp; Mechanisms, 2016. 9(2): p. 187-198.
144. Rekecki, A., et al., Effect of germ-free rearing environment on gut development of larval sea bass (Dicentrarchus labrax L.). Aquaculture, 2009. 293(1): p. 8-15.
145. Quesada, S. P., J. A. R. Paschoal, and F. G. R. Reyes, Considerations on the Aquaculture Development and on the Use of Veterinary Drugs: Special Issue for Fluoroquinolones-A Review. Journal of Food Science, 2013. 78(9): p. R1321-R1333.
146. Olivares-Fuster, O., et al., Host-specific association between Flavobacterium columnare genomovars and fish species Systematic and Applied Microbiology, 2007. 30(8): p. 624-633. 40)
147. Evenhuis, J. P., S. E. LaPatra, and D. Marancik, Early life stage rainbow trout (Oncorhynchus mykiss) mortalities due to Flavobacterium columnare in Idaho, USA. Aquaculture, 2014. 418-419: p. 126-131.
148. LaFrentz. B. R., et al., Reproducible challenge model to investigate the virulence of Flavobacterium columnare genomovars in rainbow trout Oncorhynchus mykiss. Diseases of Aquatic Organisms, 2012. 101(2): p. 115-122.
149. De Schryver, P. and O. Vadstein, Ecological theory as a foundation to control pathogenic invasion in aquaculture. The Isme Journal, 2014. 8: p. 2360.
150. Fridley, J. D., et al., THE INVASION PARADOX: RECONCILING PATTERN AND PROCESS IN SPECIES INVASIONS. Ecology, 2007. 88(1): p. 3-17
151. Zhu, J., et al., Contrasting Ecological Processes and Functional Compositions Between Intestinal Bacterial Community in Healthy and Diseased Shrimp. Microbial Ecology, 2016. 72(4): p. 975-985.
152. He, S., et al., Antibiotic growth promoter olaquindox increases pathogen susceptibility in fish by inducing gut microbiota dysbiosis. Science China Life Sciences, 2017. 60(11): p. 1260-1270.
153. Brugiroux, S., et al., Genome-guided design of a defined mouse microbiota that confers colonization resistance against Salmonella enterica serovar Typhimunum. Nat Microbiol, 2016. 2: p. 16215.
154. Kim, Y. G., et al., Neonatal acquisition of Clostridia species protects against colonization by bacterial pathogens Science, 2017. 356(6335): p. 315-319.
155. Little, A. E. F., et al., Rules of Engagement: Interspecies Interactions that Regulate Microbial Communities. Annual Review of Microbiology, 2008. 62(1): p. 375-401.
156. Stecher, B., et al., Like will to like: abundances of closely related species can predict susceptibility to intestinal colonization by pathogenic and commensal bacteria. PLOS Pathog, 2010. 6(1): p. e1000711.
157. Pérez-Sánchez, T., et al., Probiotics in aquaculture: a current assessment. Reviews in Aquaculture, 2014. 6(3): p. 133-146.
158. Huber, I., et al., Phylogenetic analysis and in situ identification of the intestinal microbial community of rainbow trout (Oncorhynchus mykiss, Walbaumn). Journal of Applied Microbiology, 2004. 96(1): p. 117-132.
159. Huyben, D., et al., Dietary live yeast and increased water temperature influence the gut microbiota of rainbow trout. Journal of Applied Microbiology, 2018. 124(6): p. 1377-1392.
160. Spanggaard, B., et al., The microflora of rainbow trout intestine: a comparison of traditional and molecular identification. Aquaculture, 2000. 182(1): p. 1-15.
161. Merrifield, D. L., et al., The current status and future focus of probiotic and prebiotic applications for salmonids. Aquaculture, 2010. 302(1): p. 1-18.
162. Schaeck, M., et al., Vibrio lentus protects gnotobiotic sea bass (Dicentrarchus labrax L.) larvae against challenge with Vibrio harveyi. Vet Microbiol, 2016. 185: p. 41-8.
163. Boutin, S., et al., Antagonistic effect of indigenous skin bacteria of brook charr (Salvelinus fontinalis) against Flavobacterium columnare and F. psychrophilum. Veterinary Microbiology, 2012. 155(2): p. 355-361.
164. Seghouani, H., et al., Walleye Autochthonous Bacteria as Promising Probiotic Candidates against Flavobacterium columnare. Frontiers in Microbiology, 2017. 8(1349).
165. Araujo, C., et al., Inhibition of fish pathogens by the microbiota from rainbow trout (Oncorhynchus mykiss, Walbaum) and rearing environment. Anaerobe, 2015. 32: p. 7-14.
166. Brunt, J. and B. Austin, Use of a probiotic to control lactococcosis and streptococcosis in rainbow trout, Oncorhynchus mykiss (Walbaum). Journal of Fish Diseases, 2005. 28(12): p. 693-701.
167. Mohammadian, T., et al., Administrations of autochthonous probiotics altered juvenile rainbow trout Oncorhynchus mykiss health status, growth performance and resistance to Lactococcus garvieae, an experimental infection. Fish & Shellfish Immunology, 2019 86: p. 269-279.
168. Pérez-Sánchez, T., et al., Identification and characterization of lactic acid bacteria isolated from rainbow trout, Oncorhynchus mykiss (Walbaum), with inhibitory activity against Lactococcus garvieae. Journal of Fish Diseases, 2011. 34(7): p. 499-507.
169. Lemon, K. P., et al., Microbiota-targeted therapies: an ecological perspective. Sci Transl Med, 2012 4(137): p. 137rv5.
170. Li. N., et al., The Type IX Secretion System Is Required for Virulence of the Fish Pathogen Flavobacterium columnare. Appl Environ Microbiol, 2017. 83(23).
171. Sato, K., et al., A protein secretion system linked to bacteroidete gliding motility and pathogenesis. Proc Natl Acad Sci USA, 2010. 107(1): p. 276-81.
172. Boirivant, M. and W. Strober, The mechanism of action of probiotics. Curr Opin Gastroenteroi, 2007. 23(6): p. 679-92.
173. Wang, C., et al., Beneficial bacteria for aquaculture: nutrition, bacteriostasis and immunoregulation J Appl Microbiol, 2019.
174. Wang, Y., et al., Two highly adhesive lactic acid bacteria strains are protective in zebrafish infected with Aeromonas hydrophila by evocation of gut mucosal immunity. Journal of Applied Microbiology, 2016. 120(2): p. 441-451.
175. Evenhuis, J. P. and B. R. LaFrentz, Virulence of Flavobacterium columnare genomnovars in rainbow trout Oncorhynchus mykiss. Diseases of Aquatic Organisms, 2016. 120(3): p. 217-224.
176. Bartelme, R. P., et al., Draft Genome Sequence of the Fish Pathogen &lt;span class=&quot;named—content genus-species&quot; id=&quot;named-content-1&quot; &gt; Flavobacterium columnare &lt;/span &gt;
Strain MS-FC-4. Genome Announcements, 2018. 6(20): p. e00429-18.
177. Vergin, K. L., et al., Screening of a Fosmid Library of Marine Environmental Genomic DNA Fragments Reveals Four Ciones Related to Members of the Order&lt;em&gt;Pianctomycetaies&lt;/em&gt. Applied and Environmental Microbiology, 1998. 64(8): p. 3075.
178. Yoon, S.-H., et al., Introducing EzBioCloud: a taxonomically united database of 16S rRNA gene sequences and whole-genome assemblies. International Journal of Systematic and Evolutionary Microbiology, 2017. 67(5): p. 1613-1617.
179. McDowell, E. M. and B. F. Trump. Histologic fixatives suitable for diagnostic light and electron microscopy. Arch Pathol Lab Med, 1976. 100(8): p. 405-14.
201. Aronesty E. Command-line tools for processing biological sequencing data. ea-utils [Internet]. 2011. Available from: https://github.com/Exprescion Analysis/ea-utils.
202. Bankevich A, Nurk S. Antipov D, Gurevich A A, Dvorkin M, Kulikov A S, et al. SPAdes: a new genome assembly algorithm and its applications to single-cell sequencing. Journal of computational biology: a journal of computational molecular cell biology. 2012; 19(5):455-77. Epub 2012/04/18. doi: 10.1089/cmb.2012.0021. PubMed PMID: 22506599; PubMed Central PMCID: PMCPMC3342519.
203. Sung-Min H, Chang Ki K, Juhye R, Jung-Hyun B, Seung-Jo Y, Seon-Bin C, et al. Application of the Whole Genome-Based Bacterial Identification System, TrueBac ID, Using Clinical isolates That Were Not Identified With Three Matrix-Assisted Laser Desorption/Ionization Time-of-Flight Mass Spectrometry (MALDI-TOF MS) Systems. Ann Lab Med. 2019; 39(6):530-6. Epub 2019/11/01. doi: 10.3343/alm.2019.39.6.530.
204. Konstantinidis K T. Tiedje J M. Genomic insights that advance the species definition for prokaryotes. Proceedings of the National Academy of Sciences of the United States of America. 2005; 102(7):2567-72 doi: 10.1073/pnas.0409727102.
205. Forberg T, Arukwe A, Vadstein O. A protocol and cultivation system for gnotobiotic Atlantic cod larvae (Gadus morhua L.) as a tool to study host microbe interactions. Aquaculture. 2011:315(3):222-7. doi:https://doi.org/10.1016/j.aquaculture.2011.02.047.
206. Declercq A M, Haesebrouck F, Van den Broeck W, Bossier P, Decostere A. Columnaris disease in fish: a review with emphasis on bacterium-host interactions. Veterinary Research. 2013; 44(1):27. doi: 10.1186/1297-9716-44-27.
207. Olivares-Fuster O. Baker J L, Terhune J S. Shoemaker C A, Klesius P H, Arias C R. Host-specific association between Flavobacterium columnare genomovars and fish species. Systematic and Applied Microbiology. 2007; 30(8): 624-33. doi: https://doi.org/10.1016/j.syapm 2007 07 003.
208. Elsabagh M, Mohamed R, Moustafa E M, Hamza A, Farrag F, Decamp O, et al. Assessing the impact of Bacillus strains mixture probiotic on water quality, growth performance, blood profile and intestinal morphology of Nile tilapia. Oreochromis niloticus. Aquaculture Nutrition. 2018; 24(6): 1613-22. doi: https://doi.org/10.1111/anu.12797.
209. Rawls J F, Samuel B S, Gordon J I. Gnotobiotic zebrafish reveal evolutionarily conserved responses to the gut microbiota. Proceedings of the National Academy of Sciences of the United States of America. 2004; 101(13):4596-601. doi: 10.1073/pnas.0400706101.
210. Bates J M, Mittge E, Kuhlman J, Baden K N, Cheesman S E, Guillemin K. Distinct signals from the microbiota promote different aspects of zebrafish gut differentiation. Developmental Biology. 2006; 297(2):374-86. doi: https://doi.org/10.1016/j.ydbio.2006.05.006.
211. Chatzidaki-Livanis M, Geva-Zatorsky N, Comstock L E. Bacteroides fragilis type VI secretion systems use novel effector and immunity proteins to antagonize human gut Bacteroidales species. Proceedings of the National Academy of Sciences. 2016; 113(13):3627. doi: 10.1073/pnas. 1522510113.
212. Bernardet J-F. Bowman J P. The Genus Flavobacterium. In: Dworkin M, Faikow S, Rosenberg E, Schleifer K-H, Stackebrandt E, editors. The Prokaryotes: Volume 7: Proteobactena: Delta, Epsilon Subclass. New York, NY: Springer New York; 2006. p. 481-531.
213. Loch T P. Faisal M. Emerging flavobacterial infections in fish: A review Journal of Advanced Research. 2015; 6(3):283-300. doi: https://doi.org/10.1016/j.jare.2014.10.009.
214. Lowrey L. Woodhams D C, Tacchi L, Salinas I. Topographical Mapping of the Rainbow Trout (Oncorhynchus mykiss) Microbiome Reveals a Diverse Bacterial Community with Antifungal Properties in the Skin. Appl Environ Microbiol. 2015; 81(19):6915-25. Epub 2015/07/26. doi: 10.1128/AEM.01826-15. PubMed PMID: 26209676; PubMed Central PMCID: PMCPMC4561705.
215. Rendueles O, Ferneres L. Fretaud M, Begaud E, Herbomel P. Levraud J P, et al. A new zebrafish model of oro-intestinal pathogen colonization reveals a key role for adhesion in protection by probiotic bacteria. PLOS Pathog. 2012; 8(7):e1002815. doi: 10.1371/journal.ppat. 1002815. PubMed PMID: 22911651; PubMed Central PMCID: PMC3406073.
216. Boirivant M, Strober W. The mechanism of action of probiotics. Curr Opin Gastroenteroi. 2007:23(6):679-92. doi: 10.1097/MOG.0b013e3282f0cffc. PubMed PMID: 17906447.
217. Wang C. Chuprom J. Wang Y, Fu L. Beneficial bacteria for aquaculture: nutrition, bacteriostasis and immunoregulation. J Appl Microbiol. 2019. Epub 2019/07/16. doi: 10.1111/jam. 14383. PubMed PMID: 31306569.
218. Aronesty E. Command-line tools for processing biological sequencing data. ea-utils [Internet]. 2011. Available from: https://github.com/ExpressionAnalysis/ea-utils.
219. Bankevich A, Nurk S, Antipov D, Gurevich A A, Dvorkin M, Kulikov A S, et al. SPAdes: a new genome assembly algorithm and its applications to single-cell sequencing. Journal of computational biology: a journal of computational molecular cell biology. 2012; 19(5):455-77. Epub 2012/04/18. doi: 10.1089/cmb.2012.0021. PubMed PMID: 22506599; PubMed Central PMCID: PMCPMC3342519.
220. Segata N, Bomigen D, Morgan X C, Huttenhower C. PhyloPhlAn is a new method for improved phylogenetic and taxonomic placement of microbes. Nat Commun. 2013; 4:2304. Epub 2013/08/15. doi: 10.1038/ncomms3304. PubMed PMID: 23942190; PubMed Central PMCID: PMCPMC3760377.
221. Stamatakis A. RAXML version 8: a tool for phylogenetic analysis and post-analysis of large phylogenies. Bioinformatics. 2014; 30(9):1312-3. Epub 2014/01/24. doi: 10.1093/bioinformatics/btu033. PubMed PMID: 24451623; PubMed Central PMCID: PMCPMC3998144.

1-18. (canceled)

19. A method comprising administering to a fish in need thereof, a probiotic active ingredient(s), wherein the probiotic active ingredient(s) is(are) a bacterial strain or combination of bacterial strains, wherein the bacterial strain or at least one bacterial strain of the combination is selected from the group consisting of: a Chryseobacterium massiliae strain, a Chryseobacterium massiliae strain wherein one or more virulence factor coding gene(s) and/or antibiotic resistance gene(s) is(are) deleted or inactivated, a Flavobacterium sp. strain whose genome has at least 95% or more Average Nucleotide Identity (ANI) with SEQ ID NO: 1 or which has at least 95% ANI with the Flavobacterium sp. strain identified by Accession Number No. I-5481 deposited at the CNCM on Jan. 24, 2020, and a Flavobacterium sp. strain whose genome has at least 95% or more Average Nucleotide Identity (ANI) with SEQ ID NO: 1 or which has at least 95% ANI with the Flavobacterium sp. strain identified by Accession Number No. I-5481 deposited at the CNCM on Jan. 24, 2020 wherein one or more virulence factor coding gene(s) and/or antibiotic resistance genes is(are) deleted or inactivated.

20. The method of claim 19, wherein the bacterial strain(s) is(are) associated with acceptable carrier or delivery vehicle(s) within a single composition or separate compositions comprising a mixture of distinct bacterial strains.

21. The method of claim 20, wherein the bacterial strain(s) is(are) further associated with adjuvant component(s)

22. The method of claim 19, wherein:
a) when only one bacterial strain is used, said bacterial strain is administered to a fish in need thereof without acceptable carrier or delivery vehicle(s), or
b) when a combination of distinct bacterial strains is used, said bacterial strains are administered to a fish in need thereof:
i) as separate bacterial strains without acceptable carrier or delivery vehicle(s), or
ii) as a mixture of distinct bacterial strains found within a single composition, or
iii) within distinct compositions each comprising at least one bacterial strain, or
iv) as a collocation of at least one individualized bacterial strain and distinct composition(s) each comprising at least one bacterial strain, and when a combination of distinct bacterial strains is used, said bacterial strains are administered to a host in need thereof simultaneously or separately in any order, or sequentially in any order.

23. The method of claim 19, wherein bacterial strain(s) is(are) selected from bacterial strain(s):
whose genome has at least 96% or more Average Nucleotide Identity (ANI) with SEQ ID NO:2 or which have at least 96% ANI with the Chryseobacterium massiliae strain identified by Accession Number No. I-5479 deposited at the CNCM on Jan. 24, 2020, and/or whose genome has at least 96% or more Average Nucleotide Identity (ANI) with SEQ ID NO: 1 or which have at least 96% ANI with the Flavobacterium sp. strain identified by Accession Number No. I-5481 deposited at the CNCM on Jan. 24, 2020, respectively, and/or
that comprise(s) a 16s rDNA sequence with at least 97% sequence identity to a 16s rDNA sequence present in a Chryseobacterium massiliae strain whose genome comprises SEQ ID NO:2 or a strain identified by Accession Number No. I-5479 deposited at the CNCM on Jan. 24, 2020, or a Flavobacterium. sp strain whose genome comprises SEQ ID NO:1 or a strain identified by Accession Number No. I-5481 deposited at the CNCM on Jan. 24, 2020, respectively, and/or
which is(are) from the group consisting of: a Chryseobacterium massiliae strain whose genome comprises SEQ ID NO:2 or a strain identified by Accession Number No. I-5479 deposited at the CNCM on Jan. 24, 2020, and a Flavobacterium sp. strain whose genome comprises SEQ ID NO:1 or a strain identified by Accession Number No. I-5481 deposited at the CNCM on Jan. 24, 2020, and/or
which is (are) variants of any one of these strains, wherein one or more virulence factor coding gene(s) and/or antibiotic resistance genes is(are) deleted or inactivated.

24. The method of claim 19, wherein the at least one bacterial strain(s) is(are) administered to:
a) a host that is a teleostean fish, or
b) hosts comprising or consisting essentially of or consisting of homogenous or mixed population(s) of fishes, for example a population(s) encompassing fishes such as rainbow trout at distinct stages of their development or growth.

25. The method of claim 24, wherein the host is a rainbow trout or a larva thereof, and when the host is a mixed population of fishes, it is a population encompassing rainbow trouts at distinct stages of their development or growth.

26. The method of claim 19, wherein the combination additionally comprises at least one another bacterial strain of the indigenous microbiota of the treated fish species.

27. The method of claim 19, wherein a composition consists essentially of at least one bacterial strain, preferably a live bacterial strain or susceptible of being revitalized and an acceptable carrier or delivery vehicle(s).

28. The method of claim 19, wherein said method:
a) prevents or minimizes infections by Flavobacterium columnare in the host fish or treated population of host fishes, or enhances the resistance against Flavobacterium columnare of the treated host fish or treated population of host fishes and/or
b) prevents or controls diseases caused by Flavobacterium columnare pathogen infections, in the treated fish species or treated population of host fishes, and/or
c) increases the lifespan of treated host fish or population of treated host fishes, or reduces the mortality of treated host fish or population of treated host fishes.

29. The method of claim 19, wherein said method prevents or minimizes infections caused by a Flavobacterium columnare pathogen or diseases caused by infections due to a Flavobacterium columnare pathogen.

30. The method of claim 29, wherein the disease is the columnaris disease.

31. The method of claim 19, wherein the at least one bacterial strain(s) is(are) administered to a teleostean fish or a population encompassing a teleostean fish selected amongst: eels (Anguilla sp.), salmonids (Oncorhynchus sp. and salmo sp.), tilapia (Oreochromis sp.), hybrid-striped bass (Morone chrysops × M. saxatilis), walleye (Stitzostedion vitreum), channel catfish, cetrachids (such as largemouth bass (Micropterus salmoides)), bait minnows (Pimephales promelas), goldfish (Carassisu auratus), carp (Cyprinus carplo) and aquarium fish (tropical fish species such as black mollies (Poecilia sphenops)) and platies (Xiphophorus maculatus), in aquaculture settings or fish husbandry settings.

32. The method of claim 19, wherein said method prevents or mitigates a rainbow trout fish disease in aquaculture settings or fish husbandry settings when a pathogen has been detected in the aquaculture settings or fish husbandry settings.

33. The method of claim 32, wherein the rainbow trout fish disease that is columnaris disease has been detected in the aquaculture settings or fish husbandry settings.

34. The method of claim 19, wherein the bacterial strain(s) or combination of bacterial strains is(are) introduced into the fish environment or introduced into the fish commensal microbial community by administration of encapsulated bacterial strain or combinations thereof.

35. The method of claim 19, wherein bacterial strain(s) or combination of bacterial strains is(are) administered to a host in need thereof in a dosage ranging from, or equivalent to, 5×10⁴ cfu/mL to 5×10⁶ cfu/mL of bacterial strain(s).

36. A probiotic composition or food product comprising at least one bacterial strain selected from the group consisting of: a Chryseobacterium massiliae strain, a Flavobacterium sp. strain with at least 95% or more Average Nucleotide Identity (ANI) value with SEQ ID NO:1 or with the Flavobacterium sp. strain identified by Accession Number No. I-5481 deposited at the CNCM on Jan. 24, 2020, and variants thereof wherein one or more virulence factor coding gene(s) and/or antibiotic resistance genes is(are) deleted or inactivated, and further acceptable carrier or delivery vehicle(s).

37. The probiotic composition or food product of claim 19, wherein the at least one bacterial strain is selected from bacterial strain(s):
1 whose genome has at least 96% or more Average Nucleotide Identity (ANI) with SEQ ID NO:2 or which have at least 96% ANI with the Chryseobacterium massiliae strain identified by Accession Number No. I-5479 deposited at the CNCM on Jan. 24, 2020, and/or whose genome has at least 96% or more Average Nucleotide Identity (ANI) with SEQ ID NO:1 or which have at least 96% ANI with the Flavobacterium sp. strain identified by Accession Number No. I-5481 deposited at the CNCM on Jan. 24, 2020, respectively, and/or
that comprise(s) a 16s rDNA sequence with at least 97% sequence identity to a 16s rDNA sequence present in a Chryseobacterium massiliae strain whose genome comprises SEQ ID NO:2 or a strain identified by Accession Number No. I-5479 deposited at the CNCM on Jan. 24, 2020, or a Flavobacterium. sp strain whose genome comprises SEQ ID NO:1 or a strain identified by Accession Number No. I-5481 deposited at the CNCM on Jan. 24, 2020, respectively, and/or
which is(are) from the group consisting of: a Chryseobacterium massiliae strain whose genome comprises SEQ ID NO:2 or a strain identified by Accession Number No. I-5479 deposited at the CNCM on Jan. 24, 2020, and a Flavobacterium sp. strain whose genome comprises SEQ ID NO:1 or a strain identified by Accession Number No. I-5481 deposited at the CNCM on Jan. 24, 2020, and/or
which is (are) variants of any one of these strains, wherein one or more virulence factor coding gene(s) and/or antibiotic resistance genes is(are) deleted or inactivated.

38. A method to identify bacterial strain(s) that are probiotic against a pathogen infection, comprising the steps of:
a) providing a germ-free trout model that is susceptible of being infected, with adverse effect on its health condition, by at least one determined pathogen, the latter of which does not infect a conventional trout with adverse effect on its health condition,
b) determining the microbiota of the said conventional trout of step a),
c) exposing the germ-free trout model to the at least one determined pathogen of step a) after reconventionalization of the germ-free trout model through inoculation of the germ-free trout with either the microbiota determined in b) or with the most represented bacterial strain(s) of said microbiota identified in b), when identified,
d) measuring the probiotic effect of the microbiota or the most represented bacterial strains thereof.
